(19) United States
(12) Patent Application Publication
(10) Pub. No.: US 2001/0006950 A1
(43) Pub. Date: Jul. 5, 2001

(54) GENETIC VACCINE VECTOR ENGINEERING

(76) Inventors: JUHA PUNNONEN, PALO ALTO, CA (US); WILLEM P.C. STEMMER, LOS GATOS, CA (US); ROBERT G. WHALEN, PARIS (FR); RUSSELL HOWARD, LOS ALTOS HILLS, CA (US)

Correspondence Address: LAW OFFICES OF JONATHAN ALAN QUINE, P O BOX 458, ALAMEDA, CA, 94501

(21) Appl. No.: 09/247,888

(22) Filed: Feb. 10, 1999

Related U.S. Application Data
(60) Provisional application No. 60/074,294, filed on Feb. 11, 1998.

(51) Int. Cl. A61K 48/00

(52) U.S. Cl. 514/44; 435/320.1

(57) ABSTRACT

This invention provides methods of obtaining improved genetic vaccines by use of DNA shuffling. Through use of the claimed methods, vectors can be obtained which exhibit increased efficacy for use as genetic vaccines. Improved vectors obtained by using the methods can have, for example, enhanced antigen expression, increased uptake into a cell, increased stability in a cell, ability to tailor an immune response, and the like.

### CROSS-REFERENCE TO RELATED APPLICATIONS

This application claims benefit of U.S. Provisional Application No. 60/074,294, filed Feb. 11, 1998, which application is incorporated herein by reference for all purposes.

### BACKGROUND OF THE INVENTION

1. Field of the Invention

This invention pertains to the field of genetic vaccines. Specifically, the invention provides multicomponent genetic vaccines that contain components that are optimized for a particular vaccination goal.

2. Background

Genetic immunization represents a novel mechanism of inducing protective humoral and cellular immunity. Vectors for genetic vaccinations generally consist of DNA that includes a promoter/enhancer sequence, the gene of interest and a polyadenylation/transcriptional terminator sequence. After intramuscular or intradermal injection, the gene of interest is expressed, followed by recognition of the resulting protein by the cells of the immune system. Genetic immunizations provide means to induce protective immunity even in situations when the pathogens are poorly characterized or cannot be isolated or cultured in laboratory environment.

Elicitation of a desired in vivo response by a genetic vaccine generally requires multiple cellular processes in a complex sequence. Several potential pathways exist along which a genetic vaccine can exert its effect on the mammalian immune system. In one pathway, the genetic vaccine vector enters cells that are the predominant cell type in the tissue that receives vaccine (e.g., muscle or epithelial cells). These cells express and release the antigen encoded by the vector. The vaccine vector can be engineered to have the antigen released as an intact protein from living transfected cells (i.e., via a secretion process) or directed to a membrane-bound form on the surface of these cells. Antigen can also be released from an intracellular compartment of such cells if those cells die. Extracellular antigen derived from any of these situations interacts with antigen presenting cells (APC) either by binding to the cell surface (specifically via IgM or via other non-immunoglobulin receptors) and subsequent endocytosis of outer membrane, or by fluid phase micropinocytosis wherein the APC internalizes extracellular fluid and its contents into an endocytic compartment. Interaction with APC may occur before or after partial proteolytic cleavage in the extracellular environment. In any case, the antigen derived from vaccine vector internalization and antigen expression within the predominant cell type in the tissue ends up within APC. The APC then process the antigen internally to prime MHC Class I and or Class II, essential steps in activation of CD4⁺T-helper cells (T_{H}1 and/or T_{H}2) and development of potent specific immune responses.

In a parallel pathway, the genetic vaccine plasmid enters APC (or the predominant cell type in the tissue) and, instead of antigen derived from plasmid expression being directed to extracellular export, antigen is proteolytically cleaved in the cell cytoplasm (in a proteasome dependent or independent process). Often, intracellular processing in such cells occurs via proteasomal degradation into peptides that are recognized by the TAP-1 and TAP-2 proteins and transported into the lumen of the rough endoplasmic reticulum (RER). The peptide fragments transported into the RER complex with MHC Class I. Such antigen fragments are then expressed on the cell surface in association with Class I. CD8⁺cytotoxic T lymphocytes (CTL) bearing specific T cell receptor then recognize the complex and can, in the presence of appropriate additional signals, differentiate into functional CTLs.

In addition, poorly characterized pathways, which are generally not dominant, exist in APC for trafficking of cytoplasmically generated peptides into endosomal compartments where they can end up complexed with MHC Class II, and thereby act to present antigen peptides to CD4⁺T_{H}1 and T_{H}2 cells. Because activation, proliferation, differentiation and immunoglobulin isotype switching by B lymphocytes requires help of CD4⁺T cells, antigen presentation in the context of MHC Class II molecules is crucial for induction of antigen-specific antibodies. By virtue of this pathway, a genetic vaccine vector can lead to CD4⁺T cell stimulation in addition to the dominant CD8⁺CTL activation process described above. This alternative pathway is, however, of little consequence in muscle cells where levels of MHC Class II expression are very low or zero.

Genetic vaccination can also elicit cytokine release from cells that bind to or take up DNA. So-called immunostimulatory or adjuvant properties of DNA are derived from its interaction with cells that internalize DNA. Cytokines can be released from cells that bind and/or internalize DNA in the absence of gene transcription. Separately, interaction of antigen with APC followed by presentation and specific recognition also stimulates release of cytokines that have positive feedback effects on these cells and other immune cells. Chief among these effects are the direction of CD4⁺T_{H} cells to differentiate/proliferate preferentially to T_{H}1 or T_{H}2 phenotypes. Furthermore, cytokines released at the site of DNA vaccination, regardless of the mechanism of their release, contribute to recruitment of other immune cells from the immediate local area and more distant sites such as draining lymph nodes. In recognition of the importance of cytokines in elicitation of a potent immune response, some investigators have included the genes for one or more cytokines in the DNA vaccine plasmid along with the target antigen for immunization. In this case cytokines are derived not only from processes intrinsic to the interaction of DNA with cells, or specific cell responses to the antigen, but via synthesis directed by the vaccine plasmid.

Immune cells are recruited to the site of immunization from distant sites or the bloodstream. Specific and non-specific immune responses are then greatly amplified. Immune cells, including APC, bearing antigen fragments complexed to MHC molecules or even expressing antigen from uptake of plasmid, also move from the immunization site to other sites (blood, hence to all tissues; lymph nodes; spleen) where additional immune recruitment and qualitative and quantitative development of the immune response ensue.

While these pathways often compete, previously available genetic vaccines have incorporated all components for influencing each of the pathways into a single polynucleotide molecule. Because separate cell types are involved in the complex interactions required for a potent immune response to a genetic vaccine vector, mutually incompatible consequences can arise from administration of a genetic vaccine that is incorporated in a single vector molecule. Current genetic vaccine vectors employ simple methods for expression of the desired antigen with few if any design elements that control the precise intracellular fate of the antigen or the immunological consequences of antigen expression. Thus, although genetic vaccines show great promise for vaccine research and development, the need for major improvements and several severe limitations of these technologies are apparent.

Largely due to the lack of suitable laboratory models, none of the existing genetic vaccine vectors have been optimized for human tissues. The existing genetic vaccine vectors typically provide low and short-lasting expression of the antigen of interest, and even large quantities of DNA do not always result in sufficiently high expression levels to induce protective immune responses. Because the mechanisms of the vector entry into the cells and transfer into the nucleus are poorly understood, virtually no attempts have been made to improve these key properties. Similarly, little is known about the mechanisms that regulate the maintenance of vector functions, including gene expression. Furthermore, although there is increasing amount of data indicating that specific sequences alter the immunostimulatory properties of the DNA, rational engineering is a very laborious and time-consuming approach when using this information to generate vector backbones with improved immunomodulatory properties.

Moreover, presently available genetic vaccine vectors do not provide sufficient stability, inducibility or levels of expression in vivo to satisfy the desire for vaccines which can deliver booster immunization without additional vaccine administration. Booster immunizations are typically required 3-4 weeks after the primary injection with existing genetic vaccines.

Therefore a need exists for improved genetic vaccine vectors and formulations, and methods for development of such vectors. The present invention fulfills these and other needs.

### SUMMARY OF THE INVENTION

The present invention provides multicomponent genetic vaccines that include at least one, and preferably two or more genetic vaccine components that confer upon the vaccine the ability to direct an immune response so as to achieve an optimal response to vaccination. For example, the genetic vaccines can include a component that provides optimal antigen release; a component that provides optimal production of cytotoxic T lymphocytes; a component that directs release of an immunomodulator; a component that directs release of a chemokine; and/or a component that facilitates binding to, or entry into, a desired target cell type. For example, a component can confer improved improves binding to, and uptake of, the genetic vaccine to target cells such as antigen-expressing cells or antigen-presenting cells.

Additional components include those that direct antigen peptides derived from uptake of an antigen into a cell to presentation on either Class I or Class II molecules. For example, one can include a component that directs antigen peptides to presentation on Class I molecules and comprises a polynucleotide that encodes a protein such as tapasin, TAP-1 and TAP-2, and/or a component that directs antigen peptides to presentation on Class II molecules and comprises a polynucleotide that encodes a protein such as an endosomal or lysosomal protease.

In some embodiments, one or more of the genetic vaccine components is obtained by a method that involves: (1) recombining at least first and second forms of a nucleic acid which can confer a desired property upon a genetic vaccine, wherein the first and second forms differ from each other in two or more nucleotides, to produce a library of recombinant nucleic acids; and (2) screening the library to identify at least one optimized recombinant component that exhibits an enhanced capacity to confer the desired property upon the genetic vaccine. If further optimization of the component is desired, the following additional steps can be conducted: (3) recombining at least one optimized recombinant component with a further form of the nucleic acid, which is the same or different from the first and second forms, to produce a further library of recombinant nucleic acids; (4) screening the further library to identify at least one further optimized recombinant component that exhibits an enhanced capacity to confer the desired property upon the genetic vaccine; and (5) repeating (3) and (4), as necessary, until the further optimized recombinant component exhibits a further enhanced capacity to confer the desired property upon the genetic vaccine.

In some embodiments of the invention, the first form of the nucleic acid is a first member of a gene family and the second form of the nucleic acid comprises a second member of the gene family. Additional forms of the module nucleic acid can also be members of the gene family. As an example, the first member of the gene family can be obtained from a first species of organism and the second member of the gene family obtained from a second species of organism. If desired, the optimized recombinant genetic vaccine component obtained by the methods of the invention can be backcrossed by, for example, recombining the optimized recombinant genetic vaccine component with a molar excess of one or both of the first and second forms of the substrate nucleic acids to produce a further library of recombinant genetic vaccine components; and screening the further library to identify at least one optimized recombinant genetic vaccine component that further enhances the capability of a genetic vaccine vector that includes the component to modulate the immune response.

Additional embodiments of the invention provide methods of obtaining a genetic vaccine component that confers upon a genetic vaccine vector an enhanced ability to replicate in a host cell. These methods involve creating a library of recombinant nucleic acids by subjecting to recombination at least two forms of a polynucleotide that can confer episomal replication upon a vector that contains the polynucleotide; introducing into a population of host cells a library of vectors, each of which contains a member of the library of recombinant nucleic acids and a polynucleotide that encodes a cell surface antigen; propagating the population of host cells for multiple generations; and identifying cells which display the cell surface antigen on a surface of the cell, wherein cells which display the cell surface antigen are likely to harbor a vector that contains a recombinant vector module which enhances the ability of the vector to replicate episomally.

Genetic vaccine components that confer upon a vector an enhanced ability to replicate in a host cell can also be obtained by creating a library of recombinant nucleic acids by subjecting to recombination at least two forms of a polynucleotide derived from a human papillomavirus that can confer episomal replication upon a vector that contains the polynucleotide; introducing a library of vectors, each of which contains a member of the library of recombinant nucleic acids, into a population of host cells; propagating the host cells for a plurality of generations; and identifying cells that contain the vector.

In additional embodiments, the invention provides methods obtaining a genetic vaccine component that confers upon a vector an enhanced ability to replicate in a human host cell by creating a library of recombinant nucleic acids by subjecting to recombination at least two forms of a polynucleotide that can confer episomal replication upon a vector that contains the polynucleotide; introducing a library of genetic vaccine vectors, each of which comprises a member of the library of recombinant nucleic acids, into a test system that mimics a human immune response; and determining whether the genetic vaccine vector replicates or induces an immune response in the test system. A suitable test system can involve human skin cells present as a xenotransplant on skin of an immunocompromised non-human host animal, for example, or a non-human mammal that comprises a functional human immune system. Replication in these systems can be detected by determining whether the animal exhibits an immune response against the antigen.

The invention also provides methods of obtaining a genetic vaccine component that confers upon a genetic vaccine an enhanced ability to enter an antigen-presenting cell. These methods involve creating a library of recombinant nucleic acids by subjecting to recombination at least two forms of a polynucleotide that can confer episomal replication upon a vector that contains the polynucleotide; introducing a library of genetic vaccine vectors, each of which comprises a member of the library of recombinant nucleic acids, into a population of antigen-presenting or antigen-processing cells; and determining the percentage of cells in the population which contain the nucleic acid vector. Antigen-presenting or antigen-processing cells of interest include, for example, B cells, monocytes/macrophages, dendritic cells, Langerhans cells, keratinocytes, and muscle cells.

In additional embodiments, the invention provides methods of obtaining a recombinant genetic vaccine component that confers upon a genetic vaccine an enhanced ability to induce a desired immune response in a mammal. These methods involve: (1) recombining at least first and second forms of a nucleic acid which comprise a genetic vaccine vector, wherein the first and second forms differ from each other in two or more nucleotides, to produce a library of recombinant genetic vaccine vectors; (2) transfecting the library of recombinant vaccine vectors into a population of mammalian cells selected from the group consisting of peripheral blood T cells, T cell clones, freshly isolated monocytes/macrophages and dendritic cells; (3) staining the cells for the presence of one or more cytokines and identifying cells which exhibit a cytokine staining pattern indicative of the desired immune response; and (4) obtaining recombinant vaccine vector nucleic acid sequences from the cells which exhibit the desired cytokine staining pattern.

Also provided by the invention are methods of improving the ability of a genetic vaccine vector to modulate an immune response by: (1) recombining at least first and second forms of a nucleic acid which comprise a genetic vaccine vector, wherein the first and second forms differ from each other in two or more nucleotides, to produce a library of recombinant genetic vaccine vectors; (2) transfecting the library of recombinant genetic vaccine vectors into a population of antigen presenting cells; and (3) isolating from the cells optimized recombinant genetic vaccine vectors which exhibit enhanced ability to modulate a desired immune response.

Another embodiment of the invention provides methods of obtaining a recombinant genetic vaccine vector that has an enhanced ability to induce a desired immune response in a mammal upon administration to the skin of the mammal. These methods involve: (1) recombining at least first and second forms of a nucleic acid which comprise a genetic vaccine vector, wherein the first and second forms differ from each other in two or more nucleotides, to produce a library of recombinant genetic vaccine vectors; (2) topically applying the library of recombinant genetic vaccine vectors to skin of a mammal; (3) identifying vectors that induce an immune response; and (4) recovering genetic vaccine vectors from the skin cells which contain vectors that induce an immune response.

The invention also provides methods of inducing an immune response in a mammal by topically applying to skin of the mammal a genetic vaccine vector, wherein the genetic vaccine vector is optimized for topical application through use of DNA shuffling. In some embodiments, the genetic vaccine is administered as a formulation selected from the group consisting of a transdermal patch, a cream, naked DNA, a mixture of DNA and a transfection-enhancing agent. Suitable transfection-enhancing agents include one or more agents selected from the group consisting of a lipid, a liposome, a protease, and a lipase. Alternatively, or in addition, the genetic vaccine can be administered after pretreatment of the skin by abrasion or hair removal.

In another embodiment, the invention provides methods of obtaining an optimized genetic vaccine component that confers upon a genetic vaccine containing the component an enhanced ability to induce or inhibit apoptosis of a cell into which the vaccine is introduced. These methods involve: (1) recombining at least first and second forms of a nucleic acid which comprise a nucleic acid that encodes an apoptosis-modulating polypeptide, wherein the first and second forms differ from each other in two or more nucleotides, to produce a library of recombinant nucleic acids; (2) transfecting the library of recombinant nucleic acids into a population of mammalian cells; (3) staining the cells for the presence of a cell membrane change which is indicative of apoptosis initiation; and (4) obtaining recombinant apoptosis-modulating genetic vaccine components from the cells which exhibit the desired apoptotic membrane changes.

Other embodiments of the invention provide methods of obtaining a genetic vaccine component that confers upon a genetic vaccine reduced susceptibility to a CTL immune response in a host mammal. These methods can involve: (1) recombining at least first and second forms of a nucleic acid which comprises a gene that encodes an inhibitor of a CTL immune response, wherein the first and second forms differ from each other in two or more nucleotides, to produce a library of recombinant CTL inhibitor nucleic acids; (2) introducing genetic vaccine vectors which comprise the library of recombinant CTL inhibitor nucleic acids into a plurality of human cells; (3) selecting cells which exhibit reduced MHC class I molecule expression; and (4) obtaining optimized recombinant CTL inhibitor nucleic acids from the selected cells.

The invention also provides methods of obtaining a genetic vaccine component that confers upon a genetic vaccine reduced susceptibility to a CTL immune response in a host mammal. These methods involve: (1) recombining at least first and second forms of a nucleic acid which comprises a gene that encodes an inhibitor of a CTL immune response, wherein the first and second forms differ from each other in two or more nucleotides, to produce a library of recombinant CTL inhibitor nucleic acids; (2) introducing viral vectors which comprise the library of recombinant CTL inhibitor nucleic acids into mammalian cells; (3) identifying mammalian cells which express a marker gene included in the viral vectors a predetermined time after introduction, wherein the identified cells are resistant to a CTL response; and (4) recovering as the genetic vaccine component the recombinant CTL inhibitor nucleic acids from the identified cells.

### BRIEF DESCRIPTION OF THE DRAWINGS

FIG. 1 shows a schematic representation of a multimodule genetic vaccine vector. A typical genetic vaccine vector will include one or more of the components indicated, each of which can be native or optimized using the DNA shuffling methods described herein. The components can be present on the same vaccine vector, or can be included in a genetic vaccine as separate molecules.

FIG. 2 shows a scheme for in vitro shuffling, “recursive sequence recombination,” of genes.

FIG. 3 shows a diagram of the application of DNA shuffling to evolution of genetic vaccines. Different forms of nucleic acids having known functional properties (e.g., regulatory, coding, and the like), are shuffled and screened to identify variants that exhibit improved properties for use as genetic vaccines.

FIG. 4 is a diagram of flow cytometry-based screening methods (FACS) for selection of optimized promoter sequences evolved using recursive shuffling. A cytomegalovirus (CMV) promoter is used for illustrative purposes.

FIG. 5 shows an apparatus that is suitable for microinjection of genetic vaccines and other reagents into tissue such as skin and muscle. The apparatus is particularly useful for screening large numbers of agents in vivo, being based on a 96-well format. The tips of the apparatus are movable to allow adjustment so that the tips fit into a microtiter plate. After obtaining a reagent of interest is obtained from a plate, the tips are adjusted to a distance of about 2-3 mm apart, enabling transfer of 96 different samples to an area of about 1.6 cm by 2.4 cm to about 2.4 cm by 3.6 cm. If desired, the volume of each sample transferred can be electronically controlled; typically the volumes transferred range from about 2 μl to about 5 μl. Each reagent can be mixed with a marker agent or dye to facilitate recognition of the injection site in the tissue. For example, gold particles of different sizes and shaped can be mixed with the reagent of interest, and microscopy and immunohistochemistry used to identify each injection site and to study the reaction induced by each reagent. When muscle tissue is injected, the injection site is first revealed by surgery.

FIG. 6 shows an example of family shuffling. Four different strains of a virus are used in this illustration, but the technique is applicable to any nucleic acid for which different strains, species, or gene families have homologous nucleic acids that have one or more nucleotide changes compared to other homologous nucleic acids. The different variant nucleic acids are shuffled as described herein, and screened or selected to identify those variants that exhibit the desired property. The shuffling and screening can be repeated one or more times to obtain further improvement.

FIG. 7 shows an example of a vector that is useful for screening to identify improved promoters from a library of shuffled promoter nucleic acids. Shuffled putative promoters are inserted into the vector upstream of a reporter gene for which expression is readily detected. For many applications, it is desirable that the product of the reporter gene be a cell surface protein so that cells which express high levels of the reporter gene can be sorted using flow cytometry-based cell sorting using the reporter gene product. Examples of suitable reporter genes include, for example, B7-2 and mAb179 epitopes. A polyadenylation region is typically placed downstream of the reporter gene (SV40 polyA is illustrated). The vector can also include a second reporter gene an internal control (GFP; green fluorescent protein); this gene is linked to a promoter (SRαₚ). The vector also typically includes a selectable marker (kanamycin/neomycin resistance is shown), and origins of replication that are functional in mammalian (SV40 ori) and/or bacterial (pUC ori) cells.

FIG. 8 shows a diagram of a scheme for cycling evolution of inducible promoters using DNA shuffling and flow cytometry-based selection. A library of shuffled promoter nucleic acids present in appropriate vectors is transfected into the cells, and those cells which exhibit the least expression of marker antigen when grown in uninduced conditions are selected. The vectors are recovered, introduced into cells, and grown in inducing conditions. Those cells that express the highest level of marker antigen are selected.

FIG. 9 provides a schematic diagram of a method for evolving a genetic vaccine vector for improved oral delivery.

FIG. 10 is an alignment of the nucleotide sequences of the immediate/early gene of two human cytomegalovirus (CMV) strains and two monkey strains.

FIG. 11 is an alignment of Intron A nucleotide sequences from CMV strains Towne and AD169.

FIG. 12 shows a schematic presentation of the promoter/enhancer/intron sequences derived from human (AD169 and Towne) and monkey (rhesus and vervet monkey) cytomegaloviruses by PCR amplification. These amplified fragments are suitable for use in family shuffling.

FIG. 13 shows the enrichment of a library by subjecting shuffled CMV promoter sequences to fluorescence-activated cell sorting.

FIG. 14 shows the functional diversity and enrichment of high activity CMV promoters obtained by DNA shuffling followed by fluorescence-activated cell sorting.

FIG. 15 shows the level of transgene expression obtained upon intramuscular injection of a vector that contained a luciferase gene under the control of a shuffled versus a control CMV promoter.

FIG. 16 shows a schematic representation of the use of DNA shuffling to generate promoter sequences in which unnecessary CpG sequences are deleted.

### DETAILED DESCRIPTION

### Definitions

The term “screening” describes, in general, a process that identifies optimal antigens. Several properties of the antigen can be used in selection and screening including antigen expression, folding, stability, immunogenicity and presence of epitopes from several related antigens. Selection is a form of screening in which identification and physical separation are achieved simultaneously by expression of a selection marker, which, in some genetic circumstances, allows cells expressing the marker to survive while other cells die (or vice versa). Screening markers include, for example, luciferase, beta-galactosidase and green fluorescent protein. Selection markers include drug and toxin resistance genes, and the like. Because of limitations in studying primary immune responses in vitro, in vivo studies are particularly useful screening methods. In these studies, the antigens are first introduced to test animals, and the immune responses are subsequently studied by analyzing protective immune responses or by studying the quality or strength of the induced immune response using lymphoid cells derived from the immunized animal. Although spontaneous selection can and does occur in the course of natural evolution, in the present methods selection is performed by man.

A “exogenous DNA segment”, “heterologous sequence” or a “heterologous nucleic acid”, as used herein, is one that originates from a source foreign to the particular host cell, or, if from the same source, is modified from its original form. Thus, a heterologous gene in a host cell includes a gene that is endogenous to the particular host cell, but has been modified. Modification of a heterologous sequence in the applications described herein typically occurs through the use of DNA shuffling. Thus, the terms refer to a DNA segment which is foreign or heterologous to the cell, or homologous to the cell but in a position within the host cell nucleic acid in which the element is not ordinarily found. Exogenous DNA segments are expressed to yield exogenous polypeptides.

The term “gene” is used broadly to refer to any segment of DNA associated with a biological function. Thus, genes include coding sequences and/or the regulatory sequences required for their expression. Genes also include nonexpressed DNA segments that, for example, form recognition sequences for other proteins. Genes can be obtained from a variety of sources, including cloning from a source of interest or synthesizing from known or predicted sequence information, and may include sequences designed to have desired parameters.

The term “isolated”, when applied to a nucleic acid or protein, denotes that the nucleic acid or protein is essentially free of other cellular components with which it is associated in the natural state. It is preferably in a homogeneous state although it can be in either a dry or aqueous solution. Purity and homogeneity are typically determined using analytical chemistry techniques such as polyacrylamide gel electrophoresis or high performance liquid chromatography. A protein which is the predominant species present in a preparation is substantially purified. In particular, an isolated gene is separated from open reading frames which flank the gene and encode a protein other than the gene of interest. The term “purified” denotes that a nucleic acid or protein gives rise to essentially one band in an electrophoretic gel. Particularly, it means that the nucleic acid or protein is at least about 50% pure, more preferably at least about 85% pure, and most preferably at least about 99% pure.

The term “naturally-occurring” is used to describe an object that can be found in nature as distinct from being artificially produced by man. For example, a polypeptide or polynucleotide sequence that is present in an organism (including viruses, bacteria, protozoa, insects, plants or mammalian tissue) that can be isolated from a source in nature and which has not been intentionally modified by man in the laboratory is naturally-occurring.

The term “nucleic acid” refers to deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g. degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer et al. (1991) Nucleic Acid Res. 19: 5081; Ohtsuka et al. (1985) J. Biol. Chem. 260: 2605-2608; Cassol et al. (1992); Rossolini et al. (1994) Mol. Cell. Probes 8: 91-98). The term nucleic acid is used interchangeably with gene, cDNA, and mRNA encoded by a gene.

“Nucleic acid derived from a gene” refers to a nucleic acid for whose synthesis the gene, or a subsequence thereof, has ultimately served as a template. Thus, an mRNA, a cDNA reverse transcribed from an mRNA, an RNA transcribed from that cDNA, a DNA amplified from the cDNA, an RNA transcribed from the amplified DNA, etc., are all derived from the gene and detection of such derived products is indicative of the presence and/or abundance of the original gene and/or gene transcript in a sample.

A nucleic acid is “operably linked” when it is placed into a functional relationship with another nucleic acid sequence. For instance, a promoter or enhancer is operably linked to a coding sequence if it increases the transcription of the coding sequence. Operably linked means that the DNA sequences being linked are typically contiguous and, where necessary to join two protein coding regions, contiguous and in reading frame. However, since enhancers generally function when separated from the promoter by several kilobases and intronic sequences may be of variable lengths, some polynucleotide elements may be operably linked but not contiguous.

A specific binding affinity between two molecules, for example, a ligand and a receptor, means a preferential binding of one molecule for another in a mixture of molecules. The binding of the molecules can be considered specific if the binding affinity is about 1×10⁴ M⁻¹ to about 1×10⁶ M⁻¹ or greater.

The term “recombinant” when used with reference to a cell indicates that the cell replicates a heterologous nucleic acid, or expresses a peptide or protein encoded by a heterologous nucleic acid. Recombinant cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques.

A “recombinant expression cassette” or simply an “expression cassette” is a nucleic acid construct, generated recombinantly or synthetically, with nucleic acid elements that are capable of effecting expression of a structural gene in hosts compatible with such sequences. Expression cassettes include at least promoters and optionally, transcription termination signals. Typically, the recombinant expression cassette includes a nucleic acid to be transcribed (e.g., a nucleic acid encoding a desired polypeptide), and a promoter. Additional factors necessary or helpful in effecting expression may also be used as described herein. For example, an expression cassette can also include nucleotide sequences that encode a signal sequence that directs secretion of an expressed protein from the host cell. Transcription termination signals, enhancers, and other nucleic acid sequences that influence gene expression, can also be included in an expression cassette.

A “multivalent antigenic polypeptide” or a “recombinant multivalent antigenic polypeptide” is a non-naturally occurring polypeptide that includes amino acid sequences from more than one source polypeptide, which source polypeptide is typically a naturally occurring polypeptide. At least some of the regions of different amino acid sequences constitute epitopes that are recognized by antibodies found in a mammal that has been injected with the source polypeptide. The source polypeptides from which the different epitopes are derived are usually homologous (i.e., have the same or a similar structure and/or function), and are often from different isolates, serotypes, strains, species, of organism or from different disease states, for example.

The terms “identical” or percent “identity,” in the context of two or more nucleic acid or polypeptide sequences, refer to two or more sequences or subsequences that are the same or have a specified percentage of amino acid residues or nucleotides that are the same, when compared and aligned for maximum correspondence, as measured using one of the following sequence comparison algorithms or by visual inspection.

The phrase “substantially identical,” in the context of two nucleic acids or polypeptides, refers to two or more sequences or subsequences that have at least 60%, preferably 80%, most preferably 90-95% nucleotide or amino acid residue identity, when compared and aligned for maximum correspondence, as measured using one of the following sequence comparison algorithms or by visual inspection. Preferably, the substantial identity exists over a region of the sequences that is at least about 50 residues in length, more preferably over a region of at least about 100 residues, and most preferably the sequences are substantially identical over at least about 150 residues. In some embodiments, the sequences are substantially identical over the entire length of the coding regions.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J Mol. Biol. 48:443 (1970), by the search for similarity method of Pearson & Lipman, Proc. Nat'l Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff& Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul (1993) Proc. Nat'l. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

Another indication that two nucleic acid sequences are substantially identical is that the two molecules hybridize to each other under stringent conditions. The phrase “hybridizing specifically to”, refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence.

“Stringent hybridization conditions” and “stringent hybridization wash conditions” in the context of nucleic acid hybridization experiments such as Southern and northern hybridizations are sequence dependent, and are different under different environmental parameters. Longer sequences hybridize specifically at higher temperatures. An extensive guide to the hybridization of nucleic acids is found in Tijssen (1993) Laboratory Techniques in Biochemistry and Molecular Biology—Hybridization with Nucleic Acid Probes part I chapter 2 “Overview of principles of hybridization and the strategy of nucleic acid probe assays”, Elsevier, N.Y. Generally, highly stringent hybridization and wash conditions are selected to be about 5° C. lower than the thermal melting point (Tₘ) for the specific sequence at a defined ionic strength and pH. Typically, under “stringent conditions” a probe will hybridize to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on a filter in a Southern or northern blot is 50% formamide with 1 mg of heparin at 42° C., with the hybridization being carried out overnight. An example of highly stringent wash conditions is 0.15 M NaCl at 72° C. for about 15 minutes. An example of stringent wash conditions is a 0.2×SSC wash at 65° C. for 15 minutes (see, Sambrook, infra., for a description of SSC buffer). Often, a high stringency wash is preceded by a low stringency wash to remove background probe signal. An example medium stringency wash for a duplex of, e.g., more than 100 nucleotides, is 1× SSC at 45° C. for 15 minutes. An example low stringency wash for a duplex of, e.g., more than 100 nucleotides, is 4-6× SSC at 40° C. for 15 minutes. For short probes (e.g., about 10 to 50 nucleotides), stringent conditions typically involve salt concentrations of less than about 1.0 M Na⁺ion, typically about 0.01 to 1.0 M Na⁺ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleic acids which do not hybridize to each other under stringent conditions are still substantially identical if the polypeptides which they encode are substantially identical. This occurs, e.g., when a copy of a nucleic acid is created using the maximum codon degeneracy permitted by the genetic code.

A further indication that two nucleic acid sequences or polypeptides are substantially identical is that the polypeptide encoded by the first nucleic acid is immunologically cross reactive with, or specifically binds to, the polypeptide encoded by the second nucleic acid. Thus, a polypeptide is typically substantially identical to a second polypeptide, for example, where the two peptides differ only by conservative substitutions.

The phrase “specifically (or selectively) binds to an antibody” or “specifically (or selectively) immunoreactive with”, when referring to a protein or peptide, refers to a binding reaction which is determinative of the presence of the protein, or an epitope from the protein, in the presence of a heterogeneous population of proteins and other biologics. Thus, under designated immunoassay conditions, the specified antibodies bind to a particular protein and do not bind in a significant amount to other proteins present in the sample. The antibodies raised against a multivalent antigenic polypeptide will generally bind to the proteins from which one or more of the epitopes were obtained. Specific binding to an antibody under such conditions may require an antibody that is selected for its specificity for a particular protein. A variety of immunoassay formats may be used to select antibodies specifically immunoreactive with a particular protein. For example, solid-phase ELISA immunoassays, Western blots, or immunohistochemistry are routinely used to select monoclonal antibodies specifically immunoreactive with a protein. See Harlow and Lane (1988) Antibodies, A Laboratory Manual, Cold Spring Harbor Publications, New York “Harlow and Lane”), for a description of immunoassay formats and conditions that can be used to determine specific immunoreactivity. Typically a specific or selective reaction will be at least twice background signal or noise and more typically more than 10 to 100 times background.

“Conservatively modified variations” of a particular polynucleotide sequence refers to those polynucleotides that encode identical or essentially identical amino acid sequences, or where the polynucleotide does not encode an amino acid sequence, to essentially identical sequences. Because of the degeneracy of the genetic code, a large number of functionally identical nucleic acids encode any given polypeptide. For instance, the codons CGU, CGC, CGA, CGG, AGA, and AGG all encode the amino acid arginine. Thus, at every position where an arginine is specified by a codon, the codon can be altered to any of the corresponding codons described without altering the encoded polypeptide. Such nucleic acid variations are “silent variations,” which are one species of “conservatively modified variations.” Every polynucleotide sequence described herein which encodes a polypeptide also describes every possible silent variation, except where otherwise noted. One of skill will recognize that each codon in a nucleic acid (except AUG, which is ordinarily the only codon for methionine) can be modified to yield a functionally identical molecule by standard techniques. Accordingly, each “silent variation” of a nucleic acid which encodes a polypeptide is implicit in each described sequence.

Furthermore, one of skill will recognize that individual substitutions, deletions or additions which alter, add or delete a single amino acid or a small percentage of amino acids (typically less than 5%, more typically less than 1%) in an encoded sequence are “conservatively modified variations” where the alterations result in the substitution of an amino acid with a chemically similar amino acid. Conservative substitution tables providing functionally similar amino acids are well known in the art. The following five groups each contain amino acids that are conservative substitutions for one another:

Aliphatic: Glycine (G), Alanine (A), Valine (V), Leucine (L), Isoleucine (I);

Aromatic: Phenylalanine (F), Tyrosine (Y), Tryptophan (W);

Sulfir-containing: Methionine (M), Cysteine (C);

Basic: Arginine (R), Lysine (K), Histidine (H);

Acidic: Aspartic acid (D), Glutamic acid (E), Asparagine (N), Glutamine (Q).

See also, Creighton (1984) Proteins, W. H. Freeman and Company, for additional groupings of amino acids. In addition, individual substitutions, deletions or additions which alter, add or delete a single amino acid or a small percentage of amino acids in an encoded sequence are also “conservatively modified variations”.

A “subsequence” refers to a sequence of nucleic acids or amino acids that comprise a part of a longer sequence of nucleic acids or amino acids (e.g., polypeptide) respectively.

### Description of the Preferred Embodiments

I. General

The present invention provides multicomponent genetic vaccines that include one or more component modules, each of which provides the genetic vaccine with the acquisition of or an improvement in a property or characteristic useful in genetic vaccination. The invention provides significant advantages over previously used genetic vaccines. Through use of a multicomponent vaccine, one can obtain an immune response that is particularly effective for a particular application. A multicomponent genetic vaccine can, for example, contain a component that is optimized for optimal antigen expression, as well as a component that confers improved activation of cytotoxic T lymphocytes (CTLs) by enhancing the presentation of the antigen on dendritic cell MHC Class I molecules. Additional examples are described herein.

In additional embodiments, the present invention provides methods of obtaining components for use in genetic vaccines, including the multicomponent vaccines, that are more effective in conferring a desired immune response property upon a genetic vaccine. The methods involve creating a library of recombinant nucleic acids and screening the library to identify those library members that exhibits an enhanced capacity to confer a desired property upon a genetic vaccine. Those recombinant nucleic acids that exhibit improved properties can be used as components in a genetic vaccine, either directly as a polynucleotide or as a protein that is obtained by expression of the component nucleic acid.

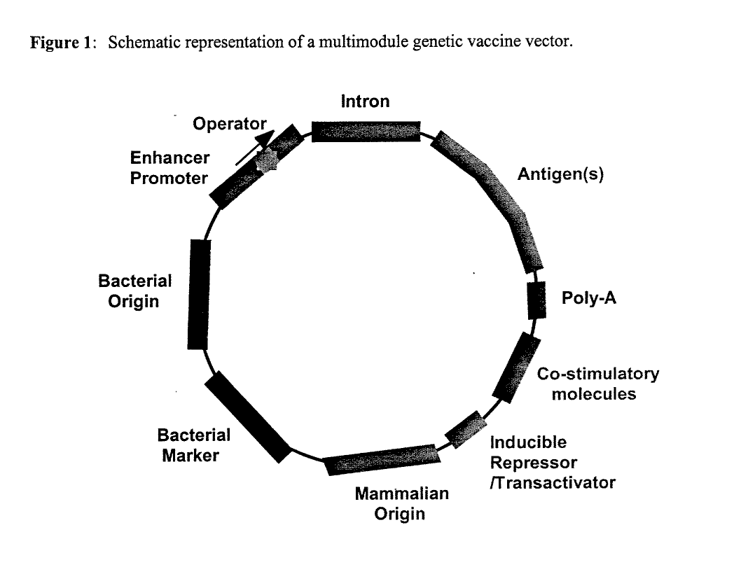

The properties or characteristics that can be sought to be acquired or improved vary widely, and, of course depend on the choice of substrate. For genetic vaccines, improvement goals include higher titer, more stable expression, improved stability, higher specificity targeting, higher or lower frequency of integration, reduced immunogenicity of the vector or an expression product thereof, increased immunogenicity of the antigen, higher expression of gene products, and the like. Other properties for which optimization is desired include the tailoring of an immune response to be most effective for a particular application. Examples of genetic vaccine components are shown in FIG. 1. Two or more components can be included in a single vector molecule, or each component can be present in a genetic vaccine formulation as a separate molecule.

In the methods of the invention, at least two variant forms of a nucleic acid are recombined to produce a library of recombinant nucleic acids, which is then screened to identify at least one recombinant component that is optimized for the particular vaccine property. Sequence recombination can be achieved in many different formats and permutations of formats, as described in further detail below. These formats share some common principles. A family of nucleic acid molecules that have some sequence identity to each other, but differ in the presence of mutations, is typically used as a substrate for recombination. In any given cycle, recombination can occur in vivo or in vitro, intracellularly or extracellularly. Furthermore, diversity resulting from recombination can be augmented in any cycle by applying prior methods of mutagenesis (e.g., error-prone PCR or cassette mutagenesis) to either the substrates or products of recombination. In some instances, a new or improved property or characteristic can be achieved after only a single cycle of in vivo or in vitro recombination, as when using different, variant forms of the sequence, as homologs from different individuals or strains of an organism, or related sequences from the same organism, as allelic variations. However, recursive sequence recombination, which entails successive cycles of recombination, can generate further improvement.

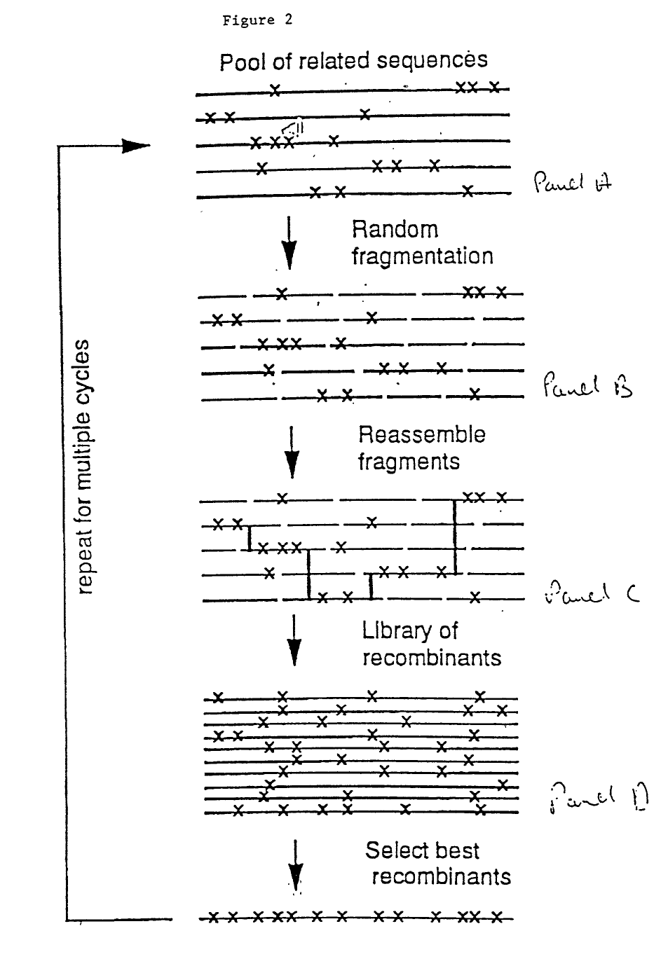

In a presently preferred embodiment, DNA shuffling is used to obtain the library of recombinant nucleic acids. DNA shuffling, which is diagrammed in FIG. 2, can result in optimization of a desired property even in the absence of a detailed understanding of the mechanism by which the particular property is mediated. The substrates for this modification, or evolution, vary in different applications, as does the property sought to be acquired or improved. Examples of candidate substrates for acquisition of a property or improvement in a property include viral and nonviral vectors used in genetic vaccination, as well as nucleic acids that are involved in mediating a particular aspect of an immune response. The methods require at least two variant forms of a starting substrate. The variant forms of candidate components can have substantial sequence or secondary structural similarity with each other, but they should also differ in at least two positions. The initial diversity between forms can be the result of natural variation, e.g., the different variant forms (homologs) are obtained from different individuals or strains of an organism (including geographic variants; termed “family shuffling”) or constitute related sequences from the same organism (e.g., allelic variations). Alternatively, the initial diversity can be induced, e.g., the second variant form can be generated by error-prone transcription, such as an error-prone PCR or use of a polymerase which lacks proof-reading activity (see, Liao (1990) Gene 88:107-111), of the first variant form, or, by replication of the first form in a mutator strain (mutator host cells are discussed in further detail below).

A recombination cycle is usually followed by at least one cycle of screening or selection for molecules having a desired property or characteristic. If a recombination cycle is performed in vitro, the products of recombination, i.e., recombinant segments, are sometimes introduced into cells before the screening step. Recombinant segments can also be linked to an appropriate vector or other regulatory sequences before screening. Alternatively, products of recombination generated in vitro are sometimes packaged as viruses before screening. If recombination is performed in vivo, recombination products can sometimes be screened in the cells in which recombination occurred. In other applications, recombinant segments are extracted from the cells, and optionally packaged as viruses, before screening.

The nature of screening or selection depends on what property or characteristic is to be acquired or the property or characteristic for which improvement is sought, and many examples are discussed below. It is not usually necessary to understand the molecular basis by which particular products of recombination (recombinant segments) have acquired new or improved properties or characteristics relative to the starting substrates. For example, a genetic vaccine vector can have many component sequences each having a different intended role (e.g., coding sequence, regulatory sequences, targeting sequences, stability-conferring sequences, immunomodulatory sequences, sequences affecting antigen presentation, and sequences affecting integration). Each of these component sequences can be varied and recombined simultaneously. Screening/selection can then be performed, for example, for recombinant segments that have increased episomal maintenance in a target cell without the need to attribute such improvement to any of the individual component sequences of the vector.

Depending on the particular screening protocol used for a desired property, initial round(s) of screening can sometimes be performed in bacterial cells due to high transfection efficiencies and ease of culture. Later rounds, and other types of screening which are not amenable to screening in bacterial cells, are generally performed in mammalian cells to optimize recombinant segments for use in an environment close to that of their intended use. Final rounds of screening can be performed in the precise cell type of intended use (e.g., a human antigen-presenting cell). In some instances, this cell can be obtained from a patient to be treated with a view, for example, to minimizing problems of immunogenicity in this patient. In some methods, use of a genetic vaccine vector in treatment can itself be used as a round of screening. That is, genetic vaccine vectors that are successively taken up and/or expressed by the intended target cells in one patient are recovered from those target cells and used to treat another patient. The genetic vaccine vectors that are recovered from the intended target cells in one patient are enriched for vectors that have evolved, i.e., have been modified by recursive recombination, toward improved or new properties or characteristics for specific uptake, immunogenicity, stability, and the like.

The screening or selection step identifies a subpopulation of recombinant segments that have evolved toward acquisition of a new or improved desired property or properties useful in genetic vaccination. Depending on the screen, the recombinant segments can be screened as components of cells, components of viruses or other vectors, or in free form. More than one round of screening or selection can be performed after each round of recombination.

If further improvement in a property is desired, at least one and usually a collection of recombinant segments surviving a first round of screening/selection are subject to a further round of recombination. These recombinant segments can be recombined with each other or with exogenous segments representing the original substrates or further variants thereof. Again, recombination can proceed in vitro or in vivo. If the previous screening step identifies desired recombinant segments as components of cells, the components can be subjected to further recombination in vivo, or can be subjected to further recombination in vitro, or can be isolated before performing a round of in vitro recombination. Conversely, if the previous screening step identifies desired recombinant segments in naked form or as components of viruses or other vectors, these segments can be introduced into cells to perform a round of in vivo recombination. The second round of recombination, irrespective how performed, generates further recombinant segments which encompass additional diversity compared to recombinant segments resulting from previous rounds.

The second round of recombination can be followed by a further round of screening/selection according to the principles discussed above for the first round. The stringency of screening/selection can be increased between rounds. Also, the nature of the screen and the property being screened for can vary between rounds if improvement in more than one property is desired or if acquiring more than one new property is desired. Additional rounds of recombination and screening can then be performed until the recombinant segments have sufficiently evolved to acquire the desired new or improved property or function.

II. Formats for Recombination

A number of different formats are available by which one can create a library of recombinant nucleic acids for screening. In some embodiments, the methods of the invention entail performing recombination (“shuffling”) and screening or selection to “evolve” individual genes, whole plasmids or viruses, multigene clusters, or even whole genomes (Stemmer (1995) Bio/Technology 13:549-553). Reiterative cycles of recombination and screening/selection can be performed to further evolve the nucleic acids of interest. Such techniques do not require the extensive analysis and computation required by conventional methods for polypeptide engineering. Shuffling allows the recombination of large numbers of mutations in a minimum number of selection cycles, in contrast to traditional, pairwise recombination events (e.g., as occur during sexual replication). Thus, the sequence recombination techniques described herein provide particular advantages in that they provide recombination between any or all of the mutations, thereby providing a very fast way of exploring the manner in which different combinations of mutations can affect a desired result. In some instances, however, structural and/or functional information is available which, although not required for sequence recombination, provides opportunities for modification of the technique.

The DNA shuffling methods can involve one or more of at least four different approaches to improve immunogenic activity as well as to broaden specificity. First, DNA-shuffling can be performed on a single gene. Secondly, several highly homologous genes can be identified by sequence comparison with known homologous genes. These genes can be synthesized and shuffled as a family of homologs, to select recombinants with the desired activity. The shuffled genes can be introduced into appropriate host cells, which can include E. coli, yeast, plants, fungi, animal cells, and the like, and those having the desired properties can be identified by the methods described herein. Third, whole genome shuffling can be performed to shuffle genes that can confer a desired property upon a genetic vaccine (along with other genomic nucleic acids). For whole genome shuffling approaches, it is not even necessary to identify which genes are being shuffled. Instead, e.g., bacterial cell or viral genomes are combined and shuffled to acquire recombinant nucleic acids that, either itself or through encoding a polypeptide, have enhanced ability to induce an immune response, as measured in any of the assays described herein. Fourth, polypeptide-encoding genes can be codon modified to access mutational diversity not present in any naturally occurring gene.

Exemplary formats and examples for sequence recombination, sometimes referred to as DNA shuffling, evolution, or molecular breeding, have been described by the present inventors and co-workers in co-pending applications U.S. patent application Ser. No. 08/198,431, filed Feb. 17, 1994, Ser. No. PCT/US95/02126, filed, Feb. 17, 1995, Ser. No. 08/425,684, filed Apr. 18, 1995, Ser. No. 08/537,874, filed Oct. 30, 1995, Ser. No. 08/564,955, filed Nov. 30, 1995, Ser. No. 08/621,859, filed Mar. 25, 1996, Ser. No. 08/621,430, filed May 25, 1996, Ser. No. PCT/US96/05480, filed Apr. 18, 1996, Ser. No. 08/650,400, filed May 20, 1996, Ser. No. 08/675,502, filed Jul. 3, 1996, Ser. No. 08/721,824, filed Sep. 27, 1996, Ser. No. PCT/US97/17300, filed Sep. 26, 1997, and Ser. No. PCT/US97/24239, filed Dec. 17, 1997; Stemmer, Science 270:1510 (1995); Stemmer et al., Gene 164:49-53 (1995); Stemmer, Bio/Technology 13:549-553 (1995); Stemmer, Proc. Natl. Acad. Sci. U.S.A. 91:10747-10751 (1994); Stemmer, Nature 370:389-391 (1994); Crameri et al., Nature Medicine 2(1):1-3 (1996); Crameri et al., Nature Biotechnology 14:315-319 (1996), each of which is incorporated by reference in its entirety for all purposes.

Other methods for obtaining libraries of recombinant polynucleotides and/or for obtaining diversity in nucleic acids used as the substrates for shuffling include, for example, homologous recombination (PCT/US98/05223; Publ. No. WO98/42727); oligonucleotide-directed mutagenesis (for review see, Smith, Ann. Rev. Genet. 19: 423-462 (1985); Botstein and Shortle, Science 229: 1193-1201 (1985); Carter, Biochem. J. 237: 1-7 (1986); Kunkel, “The efficiency of oligonucleotide directed mutagenesis” in Nucleic acids & Molecular Biology, Eckstein and Lilley, eds., Springer Verlag, Berlin (1987)). Included among these methods are oligonucleotide-directed mutagenesis (Zoller and Smith, Nucl. Acids Res. 10: 6487-6500 (1982), Methods in Enzymol. 100: 468-500 (1983), and Methods in Enzymol. 154: 329-350 (1987)) phosphothioate-modified DNA mutagenesis (Taylor et al., Nucl. Acids Res. 13: 8749-8764 (1985); Taylor et al., Nucl. Acids Res. 13: 8765-8787 (1985); Nakamaye and Eckstein, Nucl. Acids Res. 14: 9679-9698 (1986); Sayers et al, Nucl. Acids Res. 16: 791-802 (1988); Sayers et al., Nuc. Acids Res. 16: 803-814 (1988)), mutagenesis using uracil-containing templates (Kunkel, Proc. Nat'l. Acad. Sci. USA 82: 488-492 (1985) and Kunkel et al., Methods in Enzymol. 154: 367-382)); mutagenesis using gapped duplex DNA (Kramer et al., Nuc. Acids Res. 12: 9441-9456 (1984); Kramer and Fritz, Methods in Enzymol. 154: 350-367 (1987); Kramer et al., Nuc. Acids Res. 16: 7207 (1988)); and Fritz et al., Nucl. Acids Res. 16: 6987-6999 (1988)). Additional suitable methods include point mismatch repair (Kramer et al., Cell 38: 879-887 (1984)), mutagenesis using repair-deficient host strains (Carter et al., Nucl. Acids Res. 13: 4431-4443 (1985); Carter, Methods in Enzymol. 154: 382-403 (1987)), deletion mutagenesis (Eghtedarzadeh and Henikoff, Nucl. Acids Res. 14: 5115 (1986)), restriction-selection and restriction-purification (Wells et al., Phil. Trans. R. Soc. Lond. A 317: 415-423 (1986)), mutagenesis by total gene synthesis (Nambiar et al., Science 223: 1299-1301 (1984); Sakamar and Khorana, Nucl. Acids Res. 14: 6361-6372 (1988); Wells et al., Gene 34: 315-323 (1985); and Grundström et al., Nucl. Acids Res. 13: 3305-3316 (1985). Kits for mutagenesis are commercially available (e.g., Bio-Rad, Amersham International, Anglian Biotechnology).

The breeding procedure starts with at least two substrates that generally show substantial sequence identity to each other (i.e., at least about 30%, 50%, 70%, 80% or 90% sequence identity), but differ from each other at certain positions. The difference can be any type of mutation, for example, substitutions, insertions and deletions. Often, different segments differ from each other in about 5-20 positions. For recombination to generate increased diversity relative to the starting materials, the starting materials must differ from each other in at least two nucleotide positions. That is, if there are only two substrates, there should be at least two divergent positions. If there are three substrates, for example, one substrate can differ from the second at a single position, and the second can differ from the third at a different single position. The starting DNA segments can be natural variants of each other, for example, allelic or species variants. The segments can also be from nonallelic genes showing some degree of structural and usually functional relatedness (e.g., different genes within a superfamily, such as the family of Yersinia V-antigens, for example). The starting DNA segments can also be induced variants of each other. For example, one DNA segment can be produced by error-prone PCR replication of the other, the nucleic acid can be treated with a chemical or other mutagen, or by substitution of a mutagenic cassette. Induced mutants can also be prepared by propagating one (or both) of the segments in a mutagenic strain, or by inducing an error-prone repair system in the cells. In these situations, strictly speaking, the second DNA segment is not a single segment but a large family of related segments. The different segments forming the starting materials are often the same length or substantially the same length. However, this need not be the case; for example; one segment can be a subsequence of another. The segments can be present as part of larger molecules, such as vectors, or can be in isolated form.

The starting DNA segments are recombined by any of the sequence recombination formats provided herein to generate a diverse library of recombinant DNA segments. Such a library can vary widely in size from having fewer than 10 to more than 10⁵, 10⁹, 10¹² or more members. In some embodiments, the starting segments and the recombinant libraries generated will include full-length coding sequences and any essential regulatory sequences, such as a promoter and polyadenylation sequence, required for expression. In other embodiments, the recombinant DNA segments in the library can be inserted into a common vector providing sequences necessary for expression before performing screening/selection.

A further technique for recombining mutations in a nucleic acid sequence utilizes “reassembly PCR”. This method can be used to assemble multiple segments that have been separately evolved into a full length nucleic acid template such as a gene. This technique is performed when a pool of advantageous mutants is known from previous work or has been identified by screening mutants that may have been created by any mutagenesis technique known in the art, such as PCR mutagenesis, cassette mutagenesis, doped oligo mutagenesis, chemical mutagenesis, or propagation of the DNA template in vivo in mutator strains. Boundaries defining segments of a nucleic acid sequence of interest preferably lie in intergenic regions, introns, or areas of a gene not likely to have mutations of interest. Preferably, oligonucleotide primers (oligos) are synthesized for PCR amplification of segments of the nucleic acid sequence of interest, such that the sequences of the oligonucleotides overlap the junctions of two segments. The overlap region is typically about 10 to 100 nucleotides in length. Each of the segments is amplified with a set of such primers. The PCR products are then “reassembled” according to assembly protocols such as those discussed herein to assemble randomly fragmented genes. In brief, in an assembly protocol the PCR products are first purified away from the primers, by, for example, gel electrophoresis or size exclusion chromatography. Purified products are mixed together and subjected to about 1-10 cycles of denaturing, reannealing, and extension in the presence of polymerase and deoxynucleoside triphosphates (dNTP's) and appropriate buffer salts in the absence of additional primers (“self-priming”). Subsequent PCR with primers flanking the gene are used to amplify the yield of the fully reassembled and shuffled genes.

In a further embodiment, PCR primers for amplification of segments of the nucleic acid sequence of interest are used to introduce variation into the gene of interest as follows. Mutations at sites of interest in a nucleic acid sequence are identified by screening or selection, by sequencing homologues of the nucleic acid sequence, and so on. Oligonucleotide PCR primers are then synthesized which encode wild type or mutant information at sites of interest. These primers are then used in PCR mutagenesis to generate libraries of full length genes encoding permutations of wild type and mutant information at the designated positions. This technique is typically advantageous in cases where the screening or selection process is expensive, cumbersome, or impractical relative to the cost of sequencing the genes of mutants of interest and synthesizing mutagenic oligonucleotides.

III. Vectors Used in Genetic Vaccination

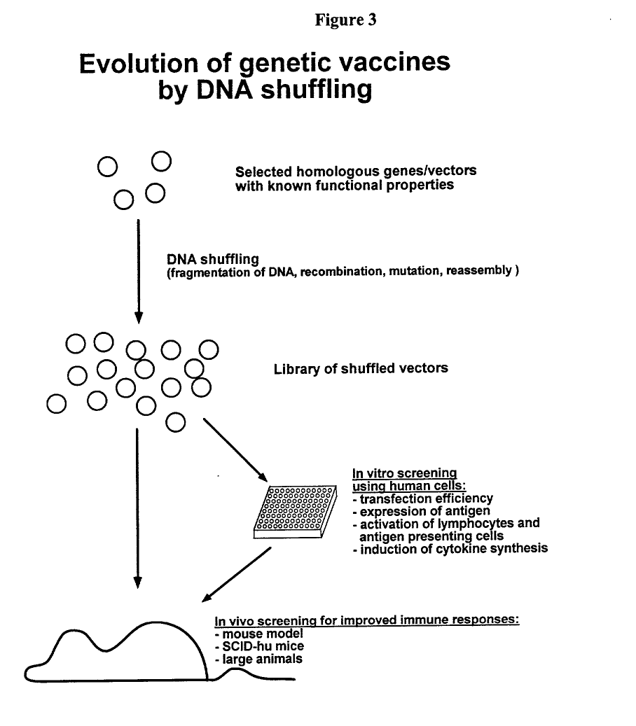

The invention provides multicomponent genetic vaccines, and methods of obtaining genetic vaccine components that improve the capability of the genetic vaccine for use in nucleic acid-mediated immunomodulation. A general approach for evolution of genetic vaccines and components by DNA shuffling is shown schematically in FIG. 3. Broadly speaking, a genetic vaccine vector is an exogenous polynucleotide which produces a medically useful phenotypic effect upon the mammalian cell(s) and organisms into which it is transferred. A vector may or may not have an origin of replication. For example, it is useful to include an origin of replication in a vector to allow for propagation of the vector in order to obtain sufficient quantities of the vector prior to administration to a patient. If the vector is designed to integrate into host chromosomal DNA or bind to host mRNA or DNA, or if replication in the host is otherwise undesirable, the origin of replication can be removed before administration, or an origin can be used that functions in the cells used for vector production but not in the target cells. However, in certain situations, including some of those discussed herein, it is desirable that the genetic vaccine vector be capable of replicating in appropriate host cells.

Vectors used in genetic vaccination can be viral or nonviral. Viral vectors are usually introduced into a patient as components of a virus. Illustrative viral vectors into which one can incorporate nucleic acids that are modified by the DNA shuffling methods of the invention include, for example, adenovirus-based vectors (Cantwell (1996) Blood 88:4676-4683; Ohashi (1997) Proc. Nat'l. Acad. Sci USA 94:1287-1292), Epstein-Barr virus-based vectors (Mazda (1997) J. Immunol. Methods 204:143-151), adenovirus-associated virus vectors, Sindbis virus vectors (Strong (1997) Gene Ther. 4: 624-627), herpes simplex virus vectors (Kennedy (1997) Brain 120: 1245-1259) and retroviral vectors (Schubert (1997) Curr. Eye Res. 16:656-662).

Nonviral vectors, typically dsDNA, can be transferred as naked DNA or associated with a transfer-enhancing vehicle, such as a receptor-recognition protein, liposome, lipoamine, or cationic lipid. This DNA can be transferred into a cell using a variety of techniques well known in the art. For example, naked DNA can be delivered by the use of liposomes which fuse with the cellular membrane or are endocytosed, i.e., by employing ligands attached to the liposome, or attached directly to the DNA, that bind to surface membrane protein receptors of the cell resulting in endocytosis. Alternatively, the cells may be permeabilized to enhance transport of the DNA into the cell, without injuring the host cells. One can use a DNA binding protein, e.g., HBGF-1, known to transport DNA into a cell. Furthermore, DNA can be delivered by bombardment of the skin by gold or other particles coated with DNA which are delivered by mechanical means, e.g., pressure. These procedures for delivering naked DNA to cells are useful in vivo. For example, by using liposomes, particularly where the liposome surface carries ligands specific for target cells, or are otherwise preferentially directed to a specific organ, one may provide for the introduction of the DNA into the target cells/organs in vivo.

A. Viral Vectors

Various viral vectors, such as retroviruses, adenoviruses, adenoassociated viruses and herpes viruses, are commonly used in genetic vaccination. They are often made up of two components, a modified viral genome and a coat structure surrounding it (see generally Smith (1995) Annu. Rev. Microbiol. 49, 807-838), although sometimes viral vectors are introduced in naked form or coated with proteins other than viral proteins. Most current viral vectors have coat structures similar to a wildtype virus. This structure packages and protects the viral nucleic acid and provides the means to bind and enter target cells. In contrast, the viral nucleic acid in a vector designed for genetic vaccination can be changed in many ways. The goals of these changes can be, for example, to enhance or reduce replication of the virus in target cells while maintaining its ability to grow in vector form in available packaging or helper cells, to incorporate new sequences that encode and enable appropriate expression of a gene of interest (e.g., an antigen-encoding gene), and to alter the immunogenicity of the viral vector itself. Viral vector nucleic acids generally comprise two components: essential cis-acting viral sequences for replication and packaging in a helper line and a transcription unit for the exogenous gene. Other viral functions can be expressed in trans in a specific packaging or helper cell line.

(1) Adenoviruses

Adenoviruses comprise a large class of nonenveloped viruses that contain linear double-stranded DNA. The normal life cycle of the virus does not require dividing cells and involves productive infection in permissive cells during which large amounts of virus accumulate. The productive infection cycle takes about 32-36 hours in cell culture and comprises two phases, the early phase, prior to viral DNA synthesis, and the late phase, during which structural proteins and viral DNA are synthesized and assembled into virions. In general, adenovirus infections are associated with mild disease in humans.

Adenovirus vectors are somewhat larger and more complex than retrovirus or AAV vectors, partly because only a small fraction of the viral genome is removed from most current vectors. If additional genes are removed, they are provided in trans to produce the vector, which so far has proved difficult. Instead, two general types of adenovirus-based vectors have been studied, E3-deletion and E1-deletion vectors. Some viruses in laboratory stocks of wild-type lack the E3 region and can growf in the absence of helper. This ability does not mean that the E3 gene products are not necessary in the wild, only that replication in cultured cells does not require them. Deletion of the E3 region allows insertion of exogenous DNA sequences to yield vectors capable of productive infection and the transient synthesis of relatively large amounts of encoded protein.

Deletion of the E1 region disables the adenovirus, but such vectors can still be grown because there exists an established human cell line (called “293”) that contains the E1 region of Ad5 and that constitutively expresses the E1 proteins. Most recent gene-therapy applications involving adenovirus have utilized E1 replacement vectors grown in 293 cells.

The main advantages of adenovirus vectors are that they are capable of efficient episomal gene transfer in a wide range of cells and tissues and that they are easy to grow in large amounts. Adenovirus-based vectors can also be used to deliver antigens after topical application onto the skin, and induction of antigen-specific immune responses can be observed following delivery to the skin (Tang et al. (1997) Nature 388: 729-730). The main disadvantage is that the host response to the virus appears to limit the duration of expression and the ability to repeat dosing, at least with high doses of first-generation vectors.

In one embodiment, the recombination methods of the invention are used to construct a novel adenovirus-phagemid capable of packaging DNA inserts over 10 kilobases in size. Incorporation of a phage fl origin in a plasmid using the methods of the invention also generates a novel in vivo shuffling format capable of evolving whole genomes of viruses, such as the 36 kb family of human adenoviruses. The widely used human adenovirus type 5 (Ad5) has a genome size of 36 kb. It is difficult to shuffle this large genome in vitro without creating an excessive number of changes which may cause a high percentage of nonviable recombinant variants. To minimize this problem and achieve whole genome shuffling of Ad5, an adenovirus-phagemid was constructed. The Ad-phagemid has been demonstrated to accept inserts as large as 15 and 24 kilobases and to effectively generate ssDNA of that size. In a further embodiment, larger DNA inserts, as large as 50 to 100 kb are inserted into the Ad-phagemid of the invention; with generation of fill length ssDNA corresponding to those large inserts. Generation of such large ssDNA fragments provides a means to evolve, i.e. modify by the recursive recombination methods of the invention, entire viral genomes. Thus, this invention provides for the first time a unique phagemid system capable of cloning large DNA inserts (>10 KB) and generating ssDNA in vitro and in vivo corresponding to those large inserts.

The genomes of related serotypes of human adenovirus are shuffled in vivo using this unique phagemid system, as described in International Application No. PCT/US97/17302 (Publ. No. WO98/13485). The genomic DNA is first cloned into a phagemid vector, and the resulting plasmid, designated an “Admid,” can be used to produce single-stranded (ss) Admid phage by using a helper M13 phage. To achieve in vivo recombination, ssAdmid phages containing the genome of homologous human adenoviruses are used to perform high multiplicity of infection (MOI) on F⁺mutS E. coli cells. The ssDNA is a better substrate for recombination enzymes such as RecA. The high MOI ensures that the probability of having multiple cross-overs between copies of the infecting ssAdmid DNA is high. The shuffled adenovirus genome is generated by purification of the double stranded Admid DNA from the infected cells and is introduction into a permissive human cell line to produce the adenovirus library. This genomic shuffling strategy is useful for creation of recombinant adenovirus variants with changes in multiple genes. This allows screening or selection of recombinant variant phenotypes resulting from combinations of variations in multiple genes.

(2) Adeno-Associated Virus (AAV)

AAV is a small, simple, nonautonomous virus containing linear single-stranded DNA. See, Muzycka, Current Topics Microbiol. Immunol. 158, 97-129 (1992). The virus requires co-infection with adenovirus or certain other viruses in order to replicate. AAV is widespread in the human population, as evidenced by antibodies to the virus, but it is not associated with any known disease. AAV genome organization is straightforward, comprising only two genes: rep and cap. The termini of the genome comprises terminal repeats (ITR) sequences of about 145 nucleotides.

AAV-based vectors typically contain only the ITR sequences flanking the transcription unit of interest. The length of the vector DNA cannot greatly exceed the viral genome length of 4680 nucleotides. Currently, growth of AAV vectors is cumbersome and involves introducing into the host cell not only the vector itself but also a plasmid encoding rep and cap to provide helper functions. The helper plasmid lacks ITRs and consequently cannot replicate and package. In addition, helper virus such as adenovirus is often required. The potential advantage of AAV vectors is that they appear capable of long-term expression in nondividing cells, possibly, though not necessarily, because the viral DNA integrates. The vectors are structurally simple, and they may therefore provoke less of a host-cell response than adenovirus.

(3) Papilloma Virus

Papillomaviruses are small, nonenveloped, icosahedral DNA viruses that replicate in the nucleus of squamous epithelial cells. Papillomaviruses consist of a single molecule of double-stranded circular DNA about 8,000 bp in size within a spherical protein coat of 72 capsomeres. Such papillomaviruses are classified by the species they infect (e.g., bovine, human, rabbit) and by type within species. Over 50 distinct human papillomaviruses (“HPV”) have been described. See, e.g., Fields Virology (3rd ed., eds. Fields et al., Lippincott-Raven, Philadelphia, 1996). Papillomaviral vectors are described in detail in copending, commonly owned U.S. patent application Ser. No. 08/958822, filed Oct. 28, 1997, which is incorporated herein by reference in its entirety for all purposes.

Papillomaviruses display a marked degree of cellular tropism for epithelial cells. Specific viral types have a preference for either cutaneous or mucosal epithelial cells. All papillomaviruses have the capacity to induce cellular proliferation. The most common clinical manifestation of proliferation is the production of benign warts. However, many papillomaviruses have capacity to be oncogenic in some individuals and some papillomaviruses are highly oncogenic. Based on the pathology of the associated lesions, most human papillomaviruses (HPVs) can be classified in one of four major groups, benign, low-risk, intermediate-risk and high-risk (Fields Virology, (Fields et al., eds., Lippincott-Raven, Philadelphia, 3d ed. 1996); DNA Tumor Viruses: Papilloma in (Encyclopedia of Cancer, Academic Press) Vol. 1, p 520-531). For example, viruses HPV-1, HPV-2, HPV-3, HPV-4, and HPV-27 are associated with benign cutaneous lesions. Viruses HPV-6 and HPV-11 are associated with vulval, penile, and laryngeal warts and are considered low-risk viruses as they are rarely associated with invasive carcinomas. Viruses HPV-16, HPV-18, HPV-31, and HPV-45 are considered high risk virus as they are associated with a high frequency with adeno- and squamous carcinoma of the cervix. Viruses HPV-5 and HPV-8 are associated with benign cutaneous lesion in a multifactorial disease Epidermodysplasia Verruciformis (EV). Such lesions, however, can progress into squamous cell carcinomas. These viruses do not fall under one of the four major risk groups. Newly discovered HPVs can classified for risk based on the frequency of cancerous lesions relative to that of HPVs that have already been classified for risk.

HPV vectors can be subjected to iterative cycles of recombination and screening (i.e., shuffling) with a view to obtaining vectors with improved properties. Improved properties include increased tissue specificity, altered tissue specificity, increased expression level, prolonged expression, increased episomal copy number, increased or decreased capacity for chromosomal integration, increased uptake capacity, and other properties as discussed herein. The starting materials for shuffling are typically vectors of the kind described above constructed from different strains of human papillomaviruses, or segments or variants of such generated by e.g., error-prone PCR or cassette mutagenesis. The human papillomaviruses, or at least the E1 and E2 coding regions thereof are preferably human cutaneous papillomaviruses.

(4) Retroviruses

Retroviruses comprise a large class of enveloped viruses that contain single-stranded RNA as the viral genome. During the normal viral life cycle, viral RNA is reverse-transcribed to yield double-stranded DNA that integrates into the host genome and is expressed over extended periods. As a result, infected cells shed virus continuously without apparent harm to the host cell. The viral genome is small (approximately 10 kb), and its prototypical organization is extremely simple, comprising three genes encoding gag, the group specific antigens or core proteins; pol, the reverse transcriptase; and env, the viral envelope protein. The termini of the RNA genome are called long terminal repeats (LTRs) and include promoter and enhancer activities and sequences involved in integration. The genome also includes a sequence required for packaging viral RNA and splice acceptor and donor sites for generation of the separate envelope mRNA. Most retroviruses can integrate only into replicating cells, although human immunodeficiency virus (HIV) appears to be an exception.

Retrovirus vectors are relatively simple, containing the 5′ and 3′LTRs, a packaging sequence, and a transcription unit composed of the gene or genes of interest, which is typically an expression cassette. To grow such a vector, one must provide the missing viral functions in trans using a so-called packaging cell line. Such a cell is engineered to contain integrated copies of gag, pol, and env but to lack a packaging signal so that no helper virus sequences become encapsidated. Additional features added to or removed from the vector and packaging cell line reflect attempts to render the vectors more efficacious or reduce the possibility of contamination by helper virus.

For some genetic vaccine applications, retroviral vectors have the advantage of being able integrate in the chromosome and therefore potentially capable of long-term expression. They can be grown in relatively large amounts, but care is needed to ensure the absence of helper virus.

B. Non-Viral Genetic Vaccine Vectors

Nonviral nucleic acid vectors used in genetic vaccination include plasmids, RNAs, polyamide nucleic acids, and yeast artificial chromosomes (YACs), and the like. Such vectors typically include an expression cassette for expressing a polypeptide against which an immune response is induced. The promoter in such an expression cassette can be constitutive, cell type-specific, stage-specific, and/or modulatable (e.g., by tetracycline ingestion; tetracycline-responsive promoter). Transcription can be increased by inserting an enhancer sequence into the vector. Enhancers are cis-acting sequences, typically between 10 to 300 base pairs in length, that increase transcription by a promoter. Enhancers can effectively increase transcription when either 5′ or 3′ to the transcription unit. They are also effective if located within an intron or within the coding sequence itself. Typically, viral enhancers are used, including SV40 enhancers, cytomegalovirus enhancers, polyoma enhancers, and adenovirus enhancers. Enhancer sequences from mammalian systems are also commonly used, such as the mouse immunoglobulin heavy chain enhancer.

Nonviral vectors encoding products useful in gene therapy can be introduced into an animal by means such as lipofection, biolistics, virosomes, liposomes, immunoliposomes, polycation:nucleic acid conjugates, naked DNA injection, artificial virions, agent-enhanced uptake of DNA, ex vivo transduction. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424, WO 91/16024. Naked DNA genetic vaccines are described in, for example, U.S. Pat. No. 5,589,486.

IV. Multicomponent Genetic Vaccines

The invention provides multicomponent genetic vaccines that are designed to obtain an optimal immune response upon administration to a mammal. In these vaccines, two or more separate genetic vaccine components are used for immunization, preferably in the same formulation. Each component can be optimized for particular functions that will occur in some cells and not in others, thus providing a means for eliciting differentiated responses in different cell types. When mutually incompatible consequences are derived from use of one plasmid, those activities are separated into different vectors that will have different fates and effects in vivo. Genetic vaccines are ideal for the formulation of several biologically active entities into one preparation. The vectors are preferably all of the same chemical type so there is no incompatibility of this nature, and can all be manufactured by the same chemical and/or biological processes. The vaccine preparation can consist of a defined molar ratio of the separate vector components that can be formulated exactly and repeatedly.

Several genetic vaccine vector components that can be used as components of a multicomponent genetic vaccine are described below. The methods of the invention greatly simplify the development of such vector components, because the mechanism by which a particular feature is controlled and the properties of a molecule that, when modified, will enhance that feature, need not be known. Even in the absence of such knowledge, by carrying out the recombination and screening methods of the invention, one can obtain vector components that are improved for each of the properties listed.

A. Vector “AR”, designed to provide optimal antigen release

Genetic vaccine vector component “AR” is designed to provide optimal release of antigen in a form that will be recognized by antigen presenting cells (APC) and taken up by those cells for efficient intracellular processing and presentation to T helper (T_{H}) cells. Cells transfected with AR plasmid can be considered as an antigen factory for APC. AR plasmids typically have one or more of the following properties, each of which can be optimized using the DNA shuffling methods of the invention:

(a) optimal plasmid binding to and uptake by the chosen antigen expressing cells (e.g., myocytes for intramuscular immunization or epithelial cells for mucosal immunization). This is a critical property which differentiates AR from other vector components in the multicomponent DNA vaccine. Optimal vector binding to the target cell includes not only the concept of very avid binding and subsequent internalization into target cells, but relative inability to bind to and enter other cells. Optimization of this ratio of desired binding to undesired binding will significantly increase the number of target cells transfected. This property can be optimized using DNA shuffling according to the present invention as described herein. For example, variant vector component sequences obtained by DNA shuffling, combinatorial assembly of vector components, insertion of random oligonucleotide sequences, and the like, can first be selected for those that bind to target cells, after which this population of cells is depleted for those that bind to other cells. Vector components for targeting genetic vaccine vectors to particular cell types, and methods of obtaining improved targeting, are described in copending, commonly assigned U.S. patent application Ser. No. ______, filed Feb. 10, 1999 as TTC Attorney Docket No. 18097-030200US, which is entitled “Targeting of Vaccine Vectors.”

(b) optimal trafficking of the vector DNA to the nucleus. Again, the present invention provides methods by which one can obtain genetic vaccine components that are optimal for such properties.

(c) optimal transcription of the antigen gene(s). This can involve, for example, the use of optimized promoters, enhancers, introns, and the like. In a preferred embodiment, cell-specific promoters are used that only allow transcription of the genes when the vector is within the nucleus of the target cell type. In this case, specificity is derived not only from selective vector entry into target cells.

(d) optimal trafficking of mRNA to the cytoplasm and optimal longevity of the mRNA in the cytoplasm. To achieve this property, the methods of the invention are used to obtain optimal 3′ and 5′ non-translated regions of the mRNA.

(e) optimal translation of the mRNA. Again, the DNA shuffling methods are used to obtain optimized recombinant sequences which exhibit optimal ribosome binding and assembly of translational machinery, plus optimal codon preference.

(f) optimal antigen structure for efficient uptake by APC. Extracellular antigen is taken up by APC by at least five non-exclusive mechanisms. One mechanism is sampling of the external fluid phase by micropinocytosis and internalization of a vesicle. The first mechanism has, as far as is presently known, no structural requirements for an antigen in the fluid phase and is therefore not relevant to considerations of designing antigen structure. A second mechanism involves binding of antigen to receptors on the APC surface; such binding occurs according to rules that are only now being studied (these receptors are not immunoglobulin family members and appear to represent several families of proteins and glycoproteins capable of binding different classes of extracellular proteins/glycoproteins). This type of binding is followed by receptor-mediated internalization, also in a vesicle. Because this mechanism is poorly understood at present, elements of antigen design cannot be incorporated in a rational design process. However, application of gene shuffling, an empirical process of selection of variant DNA molecules most successful at entry into APC, can select for variants that are improved throughout this mechanism.

The other three mechanisms all relate to specific antibody recognition of the extracellular antigen. The first mechanism involves immunoglobulin-mediated recognition of the specific antigen via IgG that is bound to Fc receptors on the cell surface. APC such as monocytes, macrophages and dendritic cells can be decorated with surface membrane IgG of diverse specificities. In a primary response, this mechanism will not be operative. In previously immunized animals, IgG on the surface of APC can specifically bind extracellular antigen and mediate uptake of the bound antigen into an intracellular endosomal compartment. Another mechanism involves binding to clonally-derived surface membrane immunoglobulin which is present on each B cells (IgM in the case of primary B cells and IgG when the animal has been previously exposed to the antigen). B cells are efficient APC. Extracellular antigen can bind specifically to surface Ig and be internalized and processed in a membrane compartment for presentation on the B cell surface. Finally, extracellular antigen can be recognized by specific soluble immunoglobulin (IgM in the case of a primary immunization and IgG in the previously immunized animals). Complexing with Ig will elicit binding to the surface of APC (via Fc receptor recognition in the case of IgG) and internalization.

In each of these latter three mechanisms, the extent to which the conformation of the antigen is the same as the recognition specificity of the pre-existing antibody is critical to the efficiency of the process of antigen presentation. Antibodies can recognize linear protein epitopes as well as conformational epitopes determined by the three dimensional structure of the protein antigen. Protective antibodies that will recognize an extracellular virus or bacterial pathogen and by binding to its surface prevent infection or mediate its immune destruction (complement mediated lysis, immune complex formation and phagocytosis) are almost exclusively generated against conformational determinants on the proteins with native structure displayed on the surface of the pathogen. Hence, it is imperative for generation of host protective humoral immunity, to have those naive B cells which bear antibody specific for conformational epitopes present on the pathogen be stimulated by direct contact with T helper cells after intracellular processing of the antigen and presentation of degradation peptides in the context of MHC Class II. This T help will allow selective proliferation of the relevant B cells with consequent mutation of antibody and antigen driven selection for antibodies with increased specificity, as well as antibody class switching.

To summarize, optimal uptake of antigen by APC to elicit humoral immunity, as well as specific CD4⁺cytotoxic T cells, requires that the antigen be in native protein conformation (as presented subsequently to the immune system upon natural infection) and recognized by naive B cells bearing the appropriate membrane antibody. Native protein conformation includes appropriate protein folding, glycosylation and any other post-translational modifications necessary for optimal reactivity with the receptors (immunoglobulin and possibly non-immunoglobulin) on APC. In addition to the three dimensional structure of the expressed antigen required for recognition by specific antibody and elicitation of the required immune responses, the structure (and sequence) can be optimized for increased protein stability outside the expressing cell, until the time when it is recognized by immune cells, including APCs. The recombination and screening methods of the invention can be used to optimize the antigen structure (and sequence) for subsequent processing after uptake by APC so that intracellular processing results in derivation of the required peptide fragments for presentation on Class I or Class II on APC and desired immune responses.

(g) optimal partitioning of the nascent antigen into the desired subcellular compartment or compartments. This can be directed by signal and trafficking signals embodied in the antigen sequence. It may be desirable for all of the antigen to be secreted from these cells; alternatively, all or part of the antigen could be directed to be expressed on the cell surface of these factory cells. Signals to direct vesicles containing the antigen to other subcellular compartments for post-translational modifications, including glycosylation, can be embodied in the antigen sequence.

(h) optimal display of the antigen on the cell surface or optimal release of the antigen from the cells. A variation on items (f) and (g) is to design the expression of the antigen within the cytoplasm of the factory cell followed by lysis of that cell to release soluble antigen. Cell death can be engineered by expression on the same genetic vaccine vector of an intracellular protein that will elicit apoptosis. In this case, the timing of cell death is balanced with the need for the cell to produce antigen, as well as the potential deleterious effect of killing some cells in a designed process.

In combination, items (a)-(h) lead to a variety of scenarios for the optimizing the longevity and extent of antigen expression. It is not always desirable that the antigen be expressed for the longest time at the highest level. In certain clinical applications, it will be important to have antigen expression that is short time-low expression, short time-high expression, long time-low expression, long time-high expression or somewhere in between.

Plasmid AR can be designed to express one or more variants of a single antigen gene or several quite different targets for immunization. Methods for obtaining optimized antigens for use in genetic vaccines are described in copending, commonly assigned U.S. patent application Ser. No. ______, filed Feb. 10, 1999 as TTC Attorney Docket No. 18097-028710 US, which is entitled “Antigen Library Immunization”. Multiple antigens can be expressed from a monocistronic or multicistronic form of the vector.

B. Vector components “CTL-DC”, “CTL-LC” and “CTL-MM”, designed for optimal production of CTLs

Genetic vector components “CTL-DC”, “CTL-LC” and “CTL-MM” are designed to direct optimal production of cytotoxic CD8⁺lymphocytes (CTL) by dendritic cells (CTL-DC), Langerhan's cells (CTL-LC), and monocytes and macrophages (CTL-MM). These vector components direct presentation of optimal antigen fragments in association with MHC Class I, thereby ensuring maximal cytotoxic T cell immune responses. Cells transfected with CTL vector components can be considered as the direct activators of this arm of specific immunity that is usually critically important for protection against viral diseases.

CTL vector components are typically designed to have one or more of the following properties, each of which can be optimized using the DNA shuffling methods of the invention:

(a) optimal vector binding to, and uptake by, the chosen antigen presenting cells (e.g., dendritic cells, monocytes/macrophages, Langerhan's cells). This is a critical property to differentiate CTL series vectors from other vectors in the multicomponent DNA vaccine. CTL series vectors preferably do not bind to or enter cells that are chosen to be the extracellular antigen expression host via AR vectors. This separation of functions is critical, as the intracellular fate and trafficking of antigen destined for stimulation of immune cells after release from an antigen expressing cell is quite different than the fate of antigen destined to be presented on the cell surface in association with MHC Class I. In the former case, antigen is directed via a signal secretion sequence to be delivered intact to the lumen of the rough endoplasmic reticulum (RER) and then secreted. In the latter case, antigen is directed to remain in the cytoplasm and there be degraded into peptide fragments by the proteasomal system followed by delivery to the lumen of the RER for association with MHC Class I. These complexes of peptide and MHC Class I are then delivered to the cell surface for specific interaction with CD8⁺cytotoxic T cells. Vector components, and methods for obtaining optimized vector components, that are optimized for targeting to desired cell types are described in copending, commonly assigned U.S. patent application Ser. No. ______, filed Feb. 10, 1999 as TTC Attorney Docket No. 18097-030200US, which is entitled “Targeting of Genetic Vaccine Vectors.”

(b) optimal transcription of the antigen gene(s). This can be accomplished by optimizing promoters, enhancers, introns, and the like, as discussed herein. Cell specific promoters are valuable in such vectors as an additional level of selectivity.

(c) optimal longevity of the mRNA. Optimal 3′ and 5′ non-translated regions of the mRNA can be obtained using the methods of the invention.

(d) optimal translation of the mRNA. Again, the DNA shuffling and selection methods of the invention can be used to obtain polynucleotide sequences for optimal ribosome binding and assembly of translational machinery, as well as optimal codon preference.

(e) optimal protein conformation. In this case, the optimal protein conformation yields appropriate cytoplasmic proteolysis and production of the correct peptides for presentation on MHC Class I and elicitation of the desired specific CTL responses, rather than a conformation that will interact with specific antibody or other receptors on the surface of APC.

(f) optimal proteolysis to generate the correct peptides. The order of specific proteolytic cleavages will depend on the nature of protein folding and the nature of proteases either in the cytoplasm or in the proteasome.

(g) optimal transport of the antigen peptides across the endoplasmic reticulum membrane to be delivered into the RER lumen. This may be mediated by recognition of the peptides by TAP proteins or by other membrane transporters.

(h) optimal association of the peptides with the Class I-β2 microglobulin complex and trafficking to the cell surface via the secretory pathway.

(i) optimal display of the MHC-peptide complex with associated accessory molecules for recognition by specific CTL.

Vector CTL can be designed to express one or more variants of a single antigen gene or several different targets for immunization. Multiple optimized antigens can be expressed from a monocistronic or multicistronic form of the vector.

C. Vectors “M”, designed for optimal release of immune modulators

Vectors “M” are designed to direct optimal release of immune modulators, such as cytokines and other growth factors, from target cells. Target cells can be either the predominant cell type in the immunized tissue or immune cells such dendritic cells (M-DC), Langerhan's cells (M-LC), monocytes & macrophages (M-MM)”. These vectors direct simultaneous expression of optimal levels of several immune cell “modulators” (cytokines, growth factors, and the like) such that the immune response is of the desired type, or combination of types, and of the desired level. Cells transfected with M vectors can be considered as the directors of the nature of the vaccine immune response (CTL vs T_{H}1 VS T_{H}2 vs NK cell, etc.) and its magnitude. The properties of these vectors reflect the nature of the cell in which the vectors are designed to operate. For example, the vectors are designed to bind to and enter the desired cell type, and/or can have cell-specific regulated promoters that drive transcription in the desired cell type. The vectors can also be engineered to direct maximal synthesis and release of the cell modulator proteins from the target cells in the desired ratio.

“M” genetic vaccine vectors are typically designed to have one or more of the following properties, each of which can be optimized using the DNA shuffling methods of the invention:

(a) optimal vector binding to and uptake by the chosen modulator expressing cell. Suitable expressing cells include, for example, muscle cells, epithelial cells or other dominant (by number) cell types in the target tissue, antigen presenting cells (e.g. dendritic cells, monocytes/macrophages, Langerhans cells). This is a critical property which differentiates M series vectors from those designed to bind to and enter other cells.

(b) optimal transcription of the immune modulator gene(s). Again, promoters, enhancers, introns, and the like can be optimized according to the methods of the invention. Cell specific promoters are very valuable here as an additional level of selectivity.

(c) optimal longevity of the mRNA. Optimal 3′ and 5′ non-translated regions of the mRNA can be obtained using the methods of the invention.

(d) optimal translation of the mRNA. Again, the DNA shuffling and selection methods of the invention can be used to obtain polynucleotide sequences for optimal ribosome binding and assembly of translational machinery, as well as optimal codon preference.

(e) optimal trafficking of the modulator into the lumen of the RER (via a signal secretion sequence). An alternative strategy for modulation of the immune response uses membrane anchored modulators rather than secretion of soluble modulator. Anchored modulator can be retained on the surface of the synthesizing cell by, for example, a hydrophobic tail and phosphoinositol glycan linkage.

(f) optimal protein conformation for each modulator. In this case, the optimal protein conformation is that which allows extracellular modulator and/or cell membrane anchored modulator to interact with the relevant receptor.

(g) the ratio of modulators and their type can be determined empirically. One will test sets of modulators that are known to work in concert to direct the immune response in the direction of a T_{H}1 response (e.g., production of IL-2 and/or IFNγ) or T_{H}2 response (e.g., IL-4, IL-5, IL-13), for example.

Vector M can be designed to express one or more modulators. Optimized immunomodulators, and methods for obtaining optimized immodulators, are described in copending, commonly assigned U.S. patent application Ser. No. ______, filed Feb. 10, 1999 as TTC Attorney Docket No. 18907-0303US, which is entitled “Optimization of Immunomodulatory Molecules.” These optimized immunomodulatory sequences are particularly suitable for use as components of the multicomponent genetic vaccines of the invention. Multiple modulators can be expressed from a monocistronic or multicistronic form of the vector.

D. Vectors “CK”; designed to direct release of chemokines

Genetic vaccine vectors designated “CK” are designed to direct optimal release of chemokines from target cells. Target cells can be either the predominant cell type in the immunized tissue, or can be immune cells such as dendritic cells (CK-DC), Langerhan's cells (CK-LC), or monocytes and macrophages (CK-MM). These vectors typically direct simultaneous expression of optimal levels of several chemokines such that the recruitment of immune cells to the site of immunization is optimal. Cells transfected with CK vectors can be considered as the traffic police, regulating the immune cells critical for the vaccine immune response. The properties of these vectors reflect the nature of the cell in which the vectors are designed to operate. For example, the vectors are designed to bind to and enter the desired cell type, and/or can have cell-specific regulated promoters that drive transcription in the desired cell type. The vectors are also engineered to direct maximal synthesis and release of the chemokines from the target cells in the desired ratio. Genetic vaccine components, and methods for obtaining components, that provide optimal release of chemokines are described in commonly assigned, copending U.S. patent application Ser. No. ______, filed Feb. 10, 1999 as TTC Attorney Docket No. 18097-0303US, entitled “Optimization of Immunomodulatory Molecules.”

CK vectors are typically designed to have one or more of the following properties, each of which can be optimized using the DNA shuffling methods of the invention:

(a) optimal vector binding to and uptake by the chosen chemokine expressing cell. Suitable cells include, for example, muscle cells, epithelial cells, or cell types that are dominant (by number) in the particular tissue of interest. Also suitable are antigen presenting cells (e.g. dendritic cells, monocytes and macrophages, Langerhans cells). This is a critical property which differentiates CK series vectors from those designed to bind to and enter other cells.

(b) optimal transcription of the chemokine gene(s). Again, promoters, enhancers, introns, and the like can be optimized according to the methods of the invention. Cell specific promoters are very valuable here as an additional level of selectivity.

(c) optimal longevity of the mRNA. Optimal 3′ and 5′ non-translated regions of the mRNA can be obtained using the methods of the invention.

(d) optimal translation of the mRNA. Again, the DNA shuffling and selection methods of the invention can be used to obtain polynucleotide sequences for optimal ribosome binding and assembly of translational machinery, as well as optimal codon preference.

(e) optimal trafficking of the chemokine into the lumen of the RER (via a signal secretion sequence). An alternative strategy for modulation of the immune response via recruitment of cells will use membrane anchored chemokine rather than secretion of soluble chemokine. Anchored chemokine will be retained on the surface of the synthesizing cell by a hydrophobic tail and phosphoinositol glycan linkage.

(f) optimal protein conformation for each chemokine. In this case, the optimal protein conformation is that which allows extracellular chemokine/cell membrane anchored chemokine to interact with the relevant receptor.

(g) the ratio of diverse chemokines can be determined empirically. One can test sets of chemokines that are known to work in concert to direct recruitment of CTL, T_{H} cells, B cells, monocytes/macrophages, eosinophils, and/or neutrophils as appropriate.

Vector CK can be designed to express one or more chemokines. Multiple chemokines can be expressed from a monocistronic or multicistronic form of the vector.

E. Other vectors

Genetic vaccines which contain one or more additional component vector moieties are also provided by the invention. For example, the genetic vaccine can include a vector that is designed to specifically enter dendritic cells and Langerhans cells, and will migrate to the draining lymph nodes. This vector is designed to provide for expression of the target antigen(s), as well as a cocktail of cytokines and chemokines relevant to elicitation of the desired immune response in the node. Depending on the clinical goals and nature of the antigen, the vector can be optimized for relatively long lived expression of the target antigen so that stimulation of the immune system is prolonged at the node. Another example is a vector that specifically modulates MHC expression in B cells. Such vectors are designed to specifically bind to and enter B cells, cells either resident in the injection site or attracted into the site. Within the B cell, this vector directs the association of antigen peptides derived from specific uptake of antigen into the endocytic compartment of the cell to either association with Class I or Class II, hence directing the elicitation of specific immunity via CD4⁺T helper cells or CD8⁺cytotoxic lymphocytes. Numerous means exist for this intracellular direction of the fate of processed peptide that are discussed herein. Examples of molecules that direct Class I presentation include tapasin, TAP-1 and TAP-2 (Koopman et al. (1997) Curr. Opin. Immunol. 9: 80-88), and those affecting Class II presentation include, for example, endosomal/lysosomal proteases (Peters (1997) Curr. Opin. Immunol. 9: 89-96). Genetic vaccine components, and methods for obtaining components, that provide optimized Class I presentation are described in commonly assigned, copending U.S. patent application Ser. No. ______, filed Feb. 10, 1999 as TTC Attorney Docket No. 18097-0303US, entitled “Optimization of Immunomodulatory Molecules.”

An optimal DNA vaccine could, for example, combine an AR vector (antigen release), a CTL-DC vector (CTL activation via dendritic cell presentation of antigen peptide on MHC Class I), an M-MM vector for release of IL-12 and IFNg from resident tissue macrophages, and a CK vector for recruitment of T_{H} cells into the immunization site.

DNA vaccination can be used for diverse goals that can include the following, among others:

stimulation of a CTL response and/or humoral response ready to react rapidly and aggressively against an invading bacterial or viral pathogen at some time in the distant future

a continuous but non-aggressive response to prevent inappropriate responses to allergens

a continuous non-aggressive and tolerization of immunity to an autoantigen in autoimmune disease

elicitation of an aggressive CTL response as rapidly as possible against tumor cell antigens

redirection of the immune response away from a strong but inappropriate immune response to an on-going chronic infection in the direction of desired responses to clear the pathogen and/or prevent pathology.

These goals cannot always be met by the format of a single vector DNA vaccine, particularly wherein competing goals are embodied within one DNA sequence. A multicomponent format allows the generation of a portfolio of DNA vaccine vectors, some of which will be reconstructed on each occasion (e.g., those vectors containing antigen) while others will be used as well characterized and understood reagents for numerous different clinical applications (e.g., the same chemokine-expressing vector can be used in different situations).

IV. Screening Assays for Optimized Genetic Vaccine Vector Modules

Recombinant nucleic acid libraries that are obtained by the methods described herein are screened to identify those DNA segments that have a property which is desirable for genetic vaccination. The particular screening assay employed will vary, as described below, depending on the particular property for which improvement is sought. Typically, the shuffled nucleic acid library is introduced into cells prior to screening. If the DNA shuffling format employed is an in vivo format, the library of recombinant DNA segments generated already exists in a cell. If the sequence recombination is performed in vitro, the recombinant library is preferably introduced into the desired cell type before screening/selection. The members of the recombinant library can be linked to an episome or virus before introduction or can be introduced directly.

A wide variety of cell types can be used as a recipient of evolved genes. Cells of particular interest include many bacterial cell types that are used to deliver vaccines or vaccine antigens (Courvalin et al.(1995) C. R. Acad. Sci. III 18: 1207-12), both gram-negative and gram-positive, such as salmonella (Attridge et al. (1997) Vaccine 15: 155-62), clostridium (Fox et al. (1996) Gene Ther. 3: 173-8), lactobacillus, shigella (Sizemore et al. (1995) Science 270: 299-302), E. coli, streptococcus (Oggioni and Pozzi (1996) Gene 169: 85-90), as well as mammalian cells, including human cells. In some embodiments of the invention, the library is amplified in a first host, and is then recovered from that host and introduced to a second host more amenable to expression, selection, or screening, or any other desirable parameter. The manner in which the library is introduced into the cell type depends on the DNA-uptake characteristics of the cell type, e.g., having viral receptors, being capable of conjugation, or being naturally competent. If the cell type is unsusceptible to natural and chemical-induced competence, but susceptible to electroporation, one would usually employ electroporation. If the cell type is unsusceptible to electroporation as well, one can employ biolistics. The biolistic PDS-1000 Gene Gun (Biorad, Hercules, Calif.) uses helium pressure to accelerate DNA-coated gold or tungsten microcarriers toward target cells. The process is applicable to a wide range of tissues, including plants, bacteria, fungi, algae, intact animal tissues, tissue culture cells, and animal embryos. One can employ electronic pulse delivery, which is essentially a mild electroporation format for live tissues in animals and patients (Zhao, Advanced Drug Delivery Reviews 17:257-262 (1995)). Novel methods for making cells competent are described in International Patent Application PCT/US97/04494 (Publ. No. WO97/35957). After introduction of the library of recombinant DNA genes, the cells are optionally propagated to allow expression of genes to occur.

In many assays, a means for identifying cells that contain a particular vector is necessary. Genetic vaccine vectors of all kinds can include a selectable marker gene. Under selective conditions, only those cells that express the selectable marker will survive. Examples of suitable markers include, the dihydrofolate reductase gene (DHFR), the thymidine kinase gene (TK), or prokaryotic genes conferring drug resistance, gpt (xanthine-guanine phosphoribosyltransferase, which can be selected for with mycophenolic acid; neo (neomycin phosphotransferase), which can be selected for with G418, hygromycin, or puromycin; and DHFR (dihydrofolate reductase), which can be selected for with methotrexate (Mulligan & Berg (1981) Proc. Nat'l Acad. Sci. USA 78: 2072; Southern & Berg (1982) J. Mol. Appl. Genet. 1: 327).

As an alternative to, or in addition to, a selectable marker, a genetic vaccine vector can include a screenable marker which, when expressed, confers upon a cell containing the vector a readily identifiable phenotype. For example, gene that encodes a cell surface antigen that is not normally present on the host cell is suitable. The detection means can be, for example, an antibody or other ligand which specifically binds to the cell surface antigen. Examples of suitable cell surface antigens include any CD (cluster of differentiation) antigen (CD1 to CD163) from a species other than that of the host cell which is not recognized by host-specific antibodies. Other examples include green fluorescent protein (GFP, see, e.g., Chalfie et al. (1994) Science 263:802-805; Crameri et al. (1996) Nature Biotechnol. 14: 315-319; Chalfie et al. (1995) Photochem. Photobiol. 62:651-656; Olson et al. (1995) J. Cell. Biol. 130:639-650) and related antigens, several of which are commercially available.

A. Screening for Vector Longevity or Translocation to Desired Tissue

For certain applications, it is desirable to identify those vectors with the greatest longevity as DNA, or to identify vectors which end up in tissues distant from the injection site. This can be accomplished by administering to an animal a population of recombinant genetic vaccine vectors by the chosen route of administration and, at various times thereafter excise the target tissue and recover vector from the tissue by standard molecular biology procedures. The recovered vector molecules can be amplified in, for example, E. coli and/or by PCR in vitro. The PCR amplification can involve further gene shuffling, after which the derived selected population used for readministration to animals and further improvement of the vector. After several rounds of this procedure, the selected vectors can be tested for their capacity to express the antigen in the correct conformation under the same conditions as the vector was selected in vivo.

Because antigen expression is not part of the selection or screening process described above, not all vectors obtained are capable of expressing the desired antigen. To overcome this drawback, the invention provides methods for identifying those vectors in a genetic vaccine population that exhibit not only the desired tissue localization and longevity of DNA integrity in vivo, but retention of maximal antigen expression (or expression of other genes such as cytokines, chemokines, cell surface accessory molecules, MHC, and the like). The methods involve in vitro identification of cells which express the desired molecule using cells purified from the tissue of choice, under conditions that allow recovery of very small numbers of cells and quantitative selection of those with different levels of antigen expression as desired.

Two embodiments of the invention are described, each of which uses a library of genetic vaccine vectors as the starting point. The goal of each method is to identify those vectors that exhibit the desired biological properties in vivo. The recombinant library represents a population of vectors that differ in known ways (e.g., a combinatorial vector library of different functional modules), or has randomly generated diversity generated either by insertion of random nucleotide stretches, or has been shuffled in vitro to introduce low level mutations across all or part of the vector.

(1) Selection for expression of cell surface-localized antigen

In a first embodiment, the invention method involves selection for expression of cell surface-localized antigen. The antigen gene is engineered in the vaccine vector library such that it has a region of amino acids which is targeted to the cell membrane. For example, the region can encode a hydrophobic stretch of C-terminal amino acids which signals the attachment of a phosphoinositol-glycan (PIG) terminus on the expressed protein and directs the protein to be expressed on the surface of the transfected cell. With an antigen that is naturally a soluble protein, this method will likely not affect the three dimensional folding of the protein in this engineered fusion with a new C-terminus. With an antigen that is naturally a transmembrane protein (e.g., a surface membrane protein on pathogenic viruses, bacteria, protozoa or tumor cells) there are at least two possibilities. First, the extracellular domain can be engineered to be in fusion with the C-terminal sequence for signaling PIG-linkage. Second, the protein can be expressed in toto relying on the signalling of the host cell to direct it efficiently to the cell surface. In a minority of cases, the antigen for expression will have an endogenous PIG terminal linkage (e.g., some antigens of pathogenic protozoa).

The vector library is delivered in vivo and, after a suitable interval of time tissue and/or cells from diverse target sites in the animal are collected. Cells can be purified from the tissue using standard cell biological procedures, including the use of cell specific surface reactive monoclonal antibodies as affinity reagents. It is relatively facile to purify isolated epithelial cells from mucosal sites where epithelium may have been inoculated or myoblasts from muscle. In some embodiments, minimal physical purification is performed prior to analysis. It is sometimes desirable to identify and separate specific cell populations from various tissues, such as spleen, liver, bone marrow, lymph node, and blood. Blood cells can be fractionated readily by FACS to separate B cells, CD4³⁰ or CD8³⁰ T cells, dendritic cells, Langerhans cells, monocytes, and the like, using diverse fluorescent monoclonal antibody reagents.

Those cells expressing the antigen can be identified with a fluorescent monoclonal antibody specific for the C-terminal sequence on PIG-linked forms of the surface antigen. FACS analysis allows quantitative assessment of the level of expression of the correct form of the antigen on the cell population. Cells expressing the maximal level of antigen are sorted and standard molecular biology methods used to recover the plasmid DNA vaccine vector that conferred this reactivity. An alternative procedure that allows purification of all those cells expressing the antigen (and that may be useful prior to loading onto a cell sorter since antigen expressing cells may be a very small minority population), is to rosette or pan-purify the cells expressing surface antigen. Rosettes can be formed between antigen expressing cells and erythrocytes bearing covalently coupled antibody to the relevant antigen. These are readily purified by unit gravity sedimentation. Panning of the cell population over petri dishes bearing immobilized monoclonal antibody specific for the relevant antigen can also be used to remove unwanted cells.

Cells expressing the required conformational structure of the target antigen can be identified using specific confornationally-dependent monoclonal antibodies that are known to react specifically with the same structure as expressed on the target pathogen. Because one monoclonal antibody cannot define all aspects of correct folding of the target antigen, one can minimize the possibility of an antigen which reacts with high affinity to the diagnostic antibody but does not yield the correct conformation as defined by that in which the antigen is found on the surface of the target pathogen or as secreted from the target pathogen. One way to minimize this possibility is to use several monoclonal antibodies, each known to react with different confornational epitopes in the correctly folded protein, in the selection process. This can be achieved by secondary FACS sorting for example.

The enriched plasmid population that successfully expressed sufficient of the antigen in the correct body site for the desired time is then used as the starting population for another round of selection, incorporating gene shuffling to expand the diversity. In this manner, one recovers the desired biological activity encoded by plasmid from tissues in DNA vaccine-immunized animals.

This method can also provide the best in vivo selected vectors that express immune accessory molecules that one may wish to incorporate into DNA vaccine constructs. For example, if it is desired to express the accessory protein B7.1 or B7.2 in antigenpresenting-cells (APC) (to promote successful presentation of antigen to T cells) one can sort APC isolated from different tissues (at or different to the inoculation site) using commercially available monoclonal antibodies that recognize functional B7 proteins.

(2) Selection for expression of secreted antigen/cytokine/chemokine

The invention also provides methods to identify plasmids in a genetic vaccine vector population that are optimal in secretion of soluble proteins that can affect the qualitative and quantitative nature of an elicited immune response. For example, the methods are useful for selecting vectors that are optimal for secretion of particular cytokines, growth factors and chemokines. The goal of the selection is to determine which particular combinations of cytokines, chemokines and growth factors, in combination with different promoters, enhancers, polyA tracts, introns, and the like, elicits the required immune response in vivo.

Combinations of the genes for the soluble proteins of interest can be present in the vectors; transcription can be either from a single promoter, or the genes can be placed in multicistronic arrangements. Typically, the genes encoding the polypeptides are present in the vaccine vector library in combination with optimal signal secretion sequences, such that the expressed proteins are secreted from the cells.

The first step in these methods is to generate vectors that are capable of secreting high (or in some case low) levels of different combinations of soluble factors in vitro and that will express those factors for a short or long time as desired. This method allows one to select for and retain an inventory of plasmids which can be characterized by known patterns of soluble protein expression in known tissues for a known time. These vectors can then be tested individually for in vivo efficacy, after being placed in combination with the genetic vaccine antigen in an appropriate expression construct.

The vector library is delivered to a test animal and, after a chosen interval of time, tissue and/or cells from diverse sites on the animal are collected. Cells are purified from the tissue using standard cell biological procedures, which often include the use of cell specific surface reactive monoclonal antibodies as affinity reagents. As is the case for cell surface antigens described above, physical purification of separate cell populations can be performed prior to identification of cells which express the desired protein. For these studies, the target cells for expression of cytokines will most usually be APC or B cells or T cells rather than muscle cells or epithelial cells. In such cases FACS sorting by established methods will be preferred to separate the different cell types. The different cell types described above may also be separated into relatively pure fractions using affinity panning, rosetting or magnetic bead separation with panels of existing monoclonal antibodies known to define the surface membrane phenotype of murine immune cells.

Purified cells are plated onto agar plates under conditions that maintain cell viability. Cells expressing the required conformational structure of the target antigen are identified using conformationally-dependent monoclonal antibodies that are known to react specifically with the same structure as expressed on the target pathogen. Release of the relevant soluble protein from the cells is detected by incubation with monoclonal antibody, followed by a secondary reagent that gives a macroscopic signal (gold deposition, color development, fluorescence, luminescence). Cells expressing the maximal level of antigen can be identified by visual inspection, the cell or cell colony picked and standard molecular biology methods used to recover the plasmid DNA vaccine vector that conferred this reactivity. Alternatively, flow cytometry can be used to identify and select cells harboring plasmids that induce high levels of gene expression. The enriched plasmid population that successfully expressed sufficient of the soluble factor in the correct body site for the desired time is then used as the starting population for another round of selection, incorporating gene shuffling to expand the diversity, if further improvement is desired. In this manner, one recovers the desired biological activity encoded by plasmid from tissues in DNA vaccine-immunized animals.

Several monoclonal antibodies, each known to react with different conformational epitopes in the correctly folded cytokine, chemokine or growth factor, can be used to confirm that the initial results from screening with one monoclonal antibody reagent still hold when several conformational epitopes are probed. In some cases the primary probe for functional cytokine released from the cell/cell colony in agar could be a soluble domain of the cognate receptor.

B. Flow Cytometry

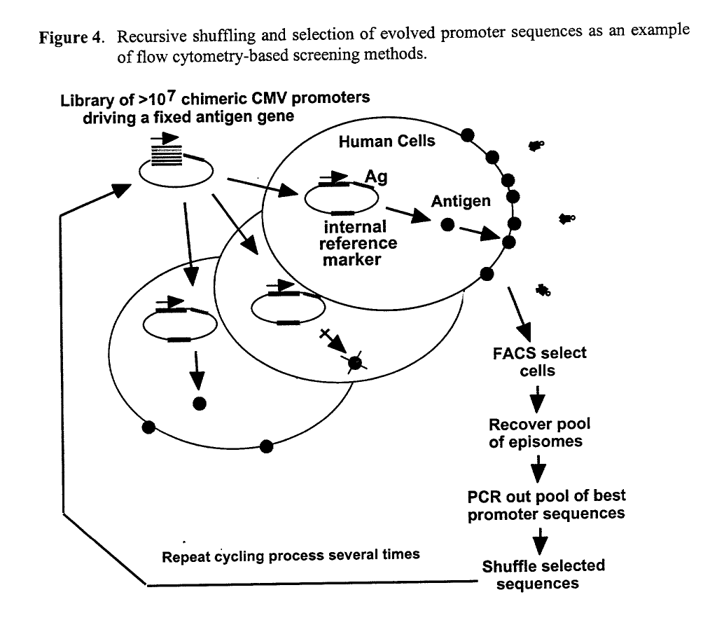

Flow cytometry provides a means to efficiently analyze the functional properties of millions of individual cells. The cells are passed through an illumination zone, where they are hit by a laser beam; the scattered light and fluorescence is analyzed by computer-linked detectors. Flow cytometry provides several advantages over other methods of analyzing cell populations. Thousands of cells can be analyzed per second, with a high degree of accuracy and sensitivity. Gating of cell populations allows multiparameter analysis of each sample. Cell size, viability, and morphology can be analyzed without the need for staining. When dyes and labeled antibodies are used, one can analyze DNA content, cell surface and intracytoplasmic proteins, and identify cell type, activation state, cell cycle stage, and detect apoptosis. Up to four colors (thus, four separate antigens stained with different fluorescent labels) and light scatter characteristics can be analyzed simultaneously (four colors requires two-laser instrument; one-laser instrument can analyze three colors). The expression levels of several genes can be analyzed simultaneously, and importantly, flow cytometry-based cell sorting (“FACS sorting”) allows selection of cells with desired phenotypes. Most of the vector module libraries, including the promoter, enhancer, intron, episomal origin of replication, expression level aspect of antigen, bacterial origin and bacterial marker, can be assayed by flow cytometry to select individual human tissue culture cells that contain the recombined nucleic acid sequences that have the greatest improvement in the desired property. Typically the selection is for high level expression of a surface antigen or surrogate marker protein, as diagrammed in FIG. 4. The pool of the best individual sequences is recovered from the cells selected by flow cytometry-based sorting. An advantage of this approach is that very large numbers (>10⁷) can be evaluated in a single vial experiment.

C. In Vitro Screening Methods

Genetic vaccine vectors and vector modules can be screened for improved vaccination properties using various in vitro testing methods that are known to those of skill in the art. For example, the optimized genetic vaccines can be tested for their effect on induction of proliferation of the particular lymphocyte type of interest, e.g., B cells, T cells, T cell lines, and T cell clones. This type of screening for improved adjuvant activity and immunostimulatory properties can be performed using, for example, human or mouse cells.

A library of genetic vaccine vectors (obtained either from shuffling of random DNA or of vectors harboring genes encoding cytokines, costimulatory molecules etc.) can be screened for cytokine production (e.g., IL-2, IL-4, IL-5, IL-6, IL-10, IL-12, IL-13, IL-15, IFN-γ, TNF-α) by B cells, T cells, monocytes/macrophages, total human PBMC, or (diluted) whole blood. Cytokines can be measured by ELISA or and cytoplasmic cytokine staining and flow cytometry (single-cell analysis). Based on the cytokine production profile, one can screen for alterations in the capacity of the vectors to direct T_{H}1/T_{H}2 differentiation (as evidenced, for example, by changes in ratios of IL-4/IFN-γ, IL-4/IL-2, IL-5/IFN-γ, IL-5/IL-2, IL-13/IFN-γ, IL-13/IL-2).

Induction of APC activation can be detected based on changes in surface expression levels of activation antigens, such as B7-1 (CD80), B7-2 (CD86), MHC class I and II, CD14, CD23, and Fc receptors, and the like.

In some embodiments, genetic vaccine vectors are analyzed for their capacity to induce T cell activation. More specifically, spleen cells from injected mice can be isolated and the capacity of cytotoxic T lymphocytes to lyse infected, autologous target cells is studied. The spleen cells are reactivated with the specific antigen in vitro. In addition, T helper cell differentiation is analyzed by measuring proliferation or production of T_{H}1 (IL-2 and IFN-γ) and T_{H}2 (IL-4 and IL-5) cytokines by ELISA and directly in CD4⁺T cells by cytoplasmic cytokine staining and flow cytometry.

Genetic vaccines and vaccine components can also be tested for ability to induce humoral immune responses, as evidenced, for example, by induction of B cell production of antibodies specific for an antigen of interest. These assays can be conducted using, for example, peripheral B lymphocytes from immunized individuals. Such assay methods are known to those of skill in the art. Other assays involve detection of antigen expression by the target cells. For example, FACS selection provides the most efficient method of identifying cells which produce a desired antigen on the cell surface. Another advantage of FACS selection is that one can sort for different levels of expression; sometimes lower expression may be desired. Another method involves panning using monoclonal antibodies on a plate. This method allows large numbers of cells to be handled in a short time, but the method only selects for highest expression levels. Capture by magnetic beads coated with monoclonal antibodies provides another method of identifying cells which express a particular antigen.

Genetic vaccines and vaccine components that are directed against cancer cells can be screened for their ability to inhibit proliferation of tumor cell lines in vitro.

Such assays are known in the art.

An indication of the efficacy of a genetic vaccine against, for example, cancer or an autoimmune disorder, is the degree of skin inflammation when the vector is injected into the skin of a patient or test animal. Strong inflammation is correlated with strong activation of antigen-specific T cells. Improved activation of tumor-specific T cells may lead to enhanced killing of the tumors. In case of autoantigens, one can add immunomodulators that skew the responses towards T_{H}2. Skin biopsies can be taken, enabling detailed studies of the type of immune response that occurs at the sites of each injection (in mice large numbers of injections/vectors can be analyzed)

Other suitable screening methods can involve detection of changes in expression of cytokines, chemokines, accessory molecules, and the like, by cells upon challenge by a library of genetic vaccine vectors.

D. Screening for Optimal Induction of Protective Immunity

To select genetic vaccine vectors that provide efficient protective immunity, one can screen the vector libraries in a test mammal using lethal infection models, such as Pseudomonas aeruginosa, Salmonella typhimurium, Escherichia coli, Klebsiella pneumoniae, Toxoplasma gondii, Plasmodium yoelii, Herpes simplex, influenza virus (e.g., Influenza A virus), and Vesicular Stomatitis Virus. Pools of genetic vaccine vectors or individual vectors are introduced into the animals intradermally, intramuscularly, intravenously, intratracheally, anally, vaginally, orally, or intraperitoneally and vectors that can prevent the disease are chosen for further rounds of shuffling and selection.

As an example, optimal vectors can be screened in mice infected with Leishmania major parasites. When injected into footpads of BALB/c mice, these parasites cause a progressive infection later resulting in a disseminated disease with fatal outcome, which can be prevented by anti-IL-4 mAbs or recombinant IL-12 (Chatelain et al. (1992) J. Immunol. 148: 1182-1187). Pools of plasmids can be injected intravenously, intraperitoneally or into footpads of these mice, and pools that can prevent the disease are chosen for further analysis and screened for vectors that can cure existing infections. The size of the footpad swelling can be followed visually providing simple yet precise monitoring of the disease progression. Mice can be infected intratracheally with Klebsiella pneumoniae resulting in lethal pneumonia, which can be prevented by recombinant IL-12 (Greenberger et al. (1996) J. Immunol. 157: 3006-3012). The advantage of this model is that the infection occurs through the lung, which is a common route of human pathogen invasion. The vectors can be given to the lung together with the pathogen or they can be administered after symptoms are evident in order to screen for vectors that can cure established infections.

In another example, the genetic vaccines are a mouse vaccination model for Influenza A virus. Influenza was one of the first models in which the efficacy of genetic vaccines was demonstrated (Ulmer et al. (1993) Science 259: 1745-1749). Several Influenza strains are lethal in mice providing an easy means to screen for efficacy of genetic vaccines. For example, Influenza virus strain A/PR/8/34, which is available through the American Type Culture Collection (ATCC VR-95), causes lethal infection, but 100% survival can be obtained when the mice are immunized with and influenza hemagglutinin (HA) genetic vaccine (Deck et al. (1997) Vaccine 15: 71-78). This model provides a way to screen for vectors that provide protection at very low quantities of DNA and/or high virus concentrations, and it also allows one to analyze the levels of antigen specific Abs and CTLs induced in vivo.

The genetic vaccine vectors can also be analyzed for their capacity to provide protection against infections by Mycobacterium tuberculosis. This is an example of a situation where genetic vaccines have provided partial protection, and where major improvements are required.

Once a number of candidate vectors has been identified, these vectors can be subjected to more detailed analysis in additional models. Testing in other infectious disease models (such as HSV, Mycoplasma pulmonis, RSV and/or rotavirus) will allow identification of vectors that are optimal in each infectious disease.

In each case, the optimal plasmids from the first round of screening can be used as the starting material for the next round of shuffling, assembly and selection. Vectors that are successful in animal models are sequenced and the corresponding human genes are cloned into genetic vaccine vectors. These vectors are then characterized in vitro for their capacity to induce differentiation of T_{H}1/T_{H}2 cells, activation of T_{H} cells, cytotoxic T lymphocytes and monocytes/macrophages, or other desired trait. Eventually, the most potent vectors, based on in vivo data in mice and comparative in vitro studies in mice and man, are chosen for human trials, and their capacity to counteract various human infectious diseases is investigated.

In addition to determining whether a vector pool provides protective immunity, one can measure immune parameters that correlate to protective immunity, such as induction of specific antibodies (particularly IgG) and induction of specific CTL responses. Spleen cells can be isolated from vaccinated mice and measured for the presence of antigen-specific T cells and induction of T_{H}1 cytokine synthesis profiles. ELISA and cytoplasmic cytokine staining, combined with flow cytometry, can provide such information on a single-cell level.

E. Screening of Genetic Vaccine Vectors that Activate Human Antigen-specific Lymphocyte Responses

To screen for vectors with optimal immunostimulatory properties for the human immune system, peripheral blood mononuclear cells (PBMCs) or purified professional antigen-presenting cells (APCs) can be isolated from previously vaccinated or infected individuals or from patients with acute infection with the pathogen of interest. Because these individuals have increased frequencies of pathogen-specific T cells in circulation, antigens expressed in PBMCs or purified APCs of these individuals will induce proliferation and cytokine production by antigen-specific CD4³⁰ and CD8³⁰ T cells. Thus, genetic vaccine vectors encoding the antigen for which the individuals have specific T cells can be transfected into PBMC of the individuals, after which induction of T cell proliferation and cytokine synthesis can be measured. Alternatively, one can screen for spontaneous entry of the genetic vaccine vector into APCs, thus providing a means by which to screen simultaneously for improved transfection efficiency, improved expression of antigen and improved induction of activation of specific T cells. Vectors with the most potent immunostimulatory properties can be screened based on their capacity to induce B cell proliferation and immunoglobulin synthesis. One buffy coat derived from a blood donor contains PBMC lymphocytes from 0.5 liters of blood, and up to 10⁴ PBMC can be obtained, enabling very large screening experiments using T cells from one donor.

When healthy vaccinated individuals (lab volunteers) are studied, one can make EBV-transformed B cell lines from these individuals. These cell lines can be used as antigen presenting cells in subsequent experiments using blood from the same donor; this reduces interassay and donor-to-donor variation). In addition, one can make antigen-specific T cell clones, after which genetic vaccines are transfected into EBV transformed B cells. The efficiency with which the transformed B cells induce proliferation of the specific T cell clones is then studied. When working with specific T cell clones, the proliferation and cytokine synthesis responses are significantly higher than when using total PBMCs, because the frequency of antigen-specific T cells among PBMC is very low.

CTL epitopes can be presented by most cells types since the class I major histocompatibility complex (MHC) surface glycoproteins are widely expressed. Therefore, transfection of cells in culture by libraries of shuffled DNA sequences in appropriate expression vectors can lead to class I epitope presentation. If specific CTLs directed to a given epitope have been isolated from an individual, then the co-culture of the transfected presenting cells and the CTLs can lead to release by the CTLs of cytokines, such as IL-2, IFN-γ, or TNFα, if the epitope is presented. Higher amounts of released TNFα. will correspond to more efficient processing and presentation of the class I epitope from the shuffled, evolved sequence.

A second method for identifying optimized CTL epitopes does not require the isolation of CTLs reacting with the epitope. In this approach, cells expressing class I MHC surface glycoproteins are transfected with the library of evolved sequences as above. After suitable incubation to allow for processing and presentations a detergent soluble extract is prepared from each cell culture and after a partial purification of the MHC-epitope complex (perhaps optional) the products are submitted to mass spectrometry (Henderson et al. (1993) Proc. Nat'l. Acad. Sci. USA 90: 10275-10279). Since the sequence is known of the epitope whose presentation to be increased, one can calibrate the mass spectrogram to identify this peptide. In addition, a cellular protein can be used for internal calibration to obtain a quantitative result; the cellular protein used for internal calibration could be the MHC molecule itself. Thus one can measure the amount of peptide epitope bound as a proportion of the MHC molecules.

F. SCID-human Skin Model for Vaccination Studies

Successful genetic vaccinations require transfection of the target cells after injection of the vector, expression of the desired antigen, processing the antigen in antigen presenting cells, presentation of the antigenic peptides in the context of MHC molecules, recognition of the peptide/MHC complex by T cell receptors, interactions of T cells with B cells and professional APCs and induction of specific T cell and B cell responses. All these events could be differentially regulated in mouse and man. A limitation of mouse models in vaccine studies is the fact that the MUC molecules of mice and man are substantially different. Therefore, proteins and peptides that effectively induce protective immune responses in mice do not necessarily function in humans.

To overcome these limitations mouse models can be used to study human tissues in mice in vivo. Live pieces of human skin are xenotransplanted onto the back of immunodeficient mice, such as SCED mice, allowing screening of the vector libraries for optimal properties in human cells in vivo. Recursive selection of episomal vectors provides strong selection pressure for vectors that remain episomal, yet provide high level of gene expression. These mice provide an excellent model for studies on transfection efficiency, transfer sequences and gene expression levels. In addition, antigen presenting cells (APCs) derived from these mice can also be used to assess the level of antigens delivered to professional APCs, and to study the capacity of these cells to present antigens and induce activation of antigen-specific CD4³⁰ and CD8³⁰ T cells in vitro. Significantly, although SCID mice have severely deficient T and B cell components, antigen presenting cells (dendritic cells and monocytes) are relatively normal in these mice.

In one embodiment of this model system, immunocompetent mice are rendered immunodeficient in order to enable transplantation of human tissue. For example, blocking of CD28 and CD40 pathways promotes long-term survival of allogeneic skin grafts in mice (Larsen et al. (1996) Nature 381: 434). Because the in vivo immunosuppression is transient, this model also enables vaccine studies in human skin xenotransplanted into mice with genetically normal immune systems. Several methods of blocking CD28-B7 interactions and CD40-CD40 ligand interactions are known to those of skill in the art, including, for example, administration of neutralizing anti-B7-1 and B7-2 antibodies, soluble CTLA-4, a soluble form of the extracellular portion of CTLA-4, a fusion protein that includes CTLA-4 and an Fc portion of an IgG molecule, and neutralizing anti-CD40 or anti-CD40 ligand antibodies. Additional methods by which one can improve transient immunosuppression include administration of one or more of the following reagents: cyclosporin A, anti-IL-2 receptor α-chain Ab, soluble IL-2 receptor, IL-10, and combinations thereof.

A model in which SCID-mice transplanted with human skin are injected with HLA-matched PBMC can be used to analyze vectors that provide long lasting expression in vivo. In this model, the vectors are injected, or topically applied, into the human skin. Thereafter, HLA-matched PBMC are injected into these mice. If the PBMC contains lymphocytes specific for the vector, the transfected cells will be recognized, and eventually destroyed, by these vector-specific lymphocytes. Therefore, this model provides possibilities to screen for vectors that efficiently escape destruction by the immune cells. It has been shown that human PBLs injected into mice with human skin transplants reject the organ, indicating that the CTLs reach the skin in this model. Obtaining HLA-matching skin and blood is possible (e.g. blood sample and skin graft from a patient undergoing skin removal due to malignancy, or blood and foreskin from the same infant).

An additional model that is suitable for screening as described herein is the modified SCIDhu mouse model, in which pieces of human fetal thymus, liver and bone marrow are transplanted into SCID mice providing functional human immune system in mice (Roncarolo et al. (1996) Semin. Immunol. 8: 207). Functional human B and T cells, and APCs can be observed in these mice. When additionally human skin is transplanted, it is likely to allow studies on the efficacy of genetic vaccine vectors following injection into the skin. Cotransplantation of skin is likely to improve the model because it will provide an additional source of professional APCs.

G. Mouse model for studying the efficiency of genetic vaccines in transfecting human muscle cells and inducing human immune responses in vivo

A lack of suitable in vivo models has hampered studies of the efficiency of genetic vaccines in inducing antigen expression in human muscle cells and in inducing specific human immune responses. The vast majority of studies on the capacity of genetic vaccines to transfect muscle cells and to induce specific immune responses in vivo have employed a mouse model. Because of the complexity of events occurring after genetic vaccination, however, it is sometimes difficult to predict whether results obtained in the mouse model reliably predict the outcome of similar vaccinations in humans. The events required in successful genetic vaccination include transfection of the cells after delivery of the plasmid, expression of the desired antigen, processing the antigen in antigen presenting cells, presentation of the antigenic peptides in the context of MHC molecules, recognition of the peptide/MHC complex by T cell receptors, interactions of T cells with B cells and professional antigen presenting cells and finally induction of specific T cell and B cell responses. All these events are likely to be somewhat differentially regulated in mouse and man.

The invention provides an in vivo model for human muscle cell transfection. This model system is especially valuable because there is no in vitro culture system available for normal muscle cells. Muscle tissue, obtained for example from cadavers, is transplanted subcutaneously into immunodeficient mice. Immunodeficient mice can be transplanted with tissues from other species without rejection. Mice suitable for xenotransplantations include, but are not limited to, SCID mice, nude mice and mice rendered deficient in their genes encoding RAG1 or RAG2 genes. SCID mice and RAG deficient mice lack functional T and B cells, and therefore are severely immunocompromised and are unable to reject transplanted organs. Previous studies indicate that these mice can be transplanted with human tissues, such as skin, spleen, liver, thymus or bone, without rejection (Roncarolo et al. (1996) Semin. Immunol. 8: 207). After transplantation of human fetal lymphoid tissues into SCID mice, functional human immune system can be demonstrated in these mice, a model generally referred to as SCID-hu mice. When human muscle tissue is transplanted into SCID-hu mice, one can not only study transfection efficiency and expression of the desired antigen, but one can also study induction of specific human immune responses induced by genetic vaccines in vivo. In this case, muscle and lymphoid organs from the same donor are used. Fetal muscle also has an advantage in that it contains few mature lymphocytes of donor origin decreasing likelihood of graft versus host reaction.

Once the human muscle tissue is established in the mouse, genetic vaccine vectors are introduced into the human muscle tissue to study the expression of the antigen of interest. When studying transfection efficiency only, RAG deficient mice are preferred, because these mice never have mature B or T cells in the circulation, whereas “leakiness” of SCID phenotype has been demonstrated which may cause variation in the transplantation efficiency.

The survival of human muscle tissue in mice is likely to be limited even in immunocompromised mice. However, because expression studies can be performed within one or two days, this model provides an efficient means to study gene expression in human muscle cells in vivo. A modified SCID-hu mouse model with human muscle transplanted into these mice can be used to study human immune responses in mice in vivo.

H. Screening for Improved Delivery of Vaccines

For certain applications, it is desirable to identify genetic vaccine vectors that are capable of being administered in a particular manner, for example, orally or through the skin. The following screening methods provide suitable assays; additional assays are also described herein in conjunction with particular genetic vaccine properties for which the assays are especially suitable.

Screening for oral delivery can be performed either in vitro or in vivo. An example of an in vitro method is based on Caco-2 (human colon adenocarcinoma) cells which are grown in tissue culture. When grown on semipermeable filters, these cells spontaneously differentiate into cells that resemble human small intestine epithelium, both structurally and functionally. Genetic vaccine libraries and/or vectors can be placed on one side of the Caco-2 cell layer, and vectors that are able to move through the cell layer are detected on the opposite side of the layer.

Libraries can also be screened for amenability to oral delivery in vivo. For example, a library of vectors can be administered orally, after which target tissues are assayed for presence of vectors. Intestinal epithelium, liver, and the bloodstream are examples of tissues that can be tested for presence of library members. Vectors that are successful in reaching the target tissue can be recovered and, if further improvement is desired, used in succeeding rounds of shuffling and selection.

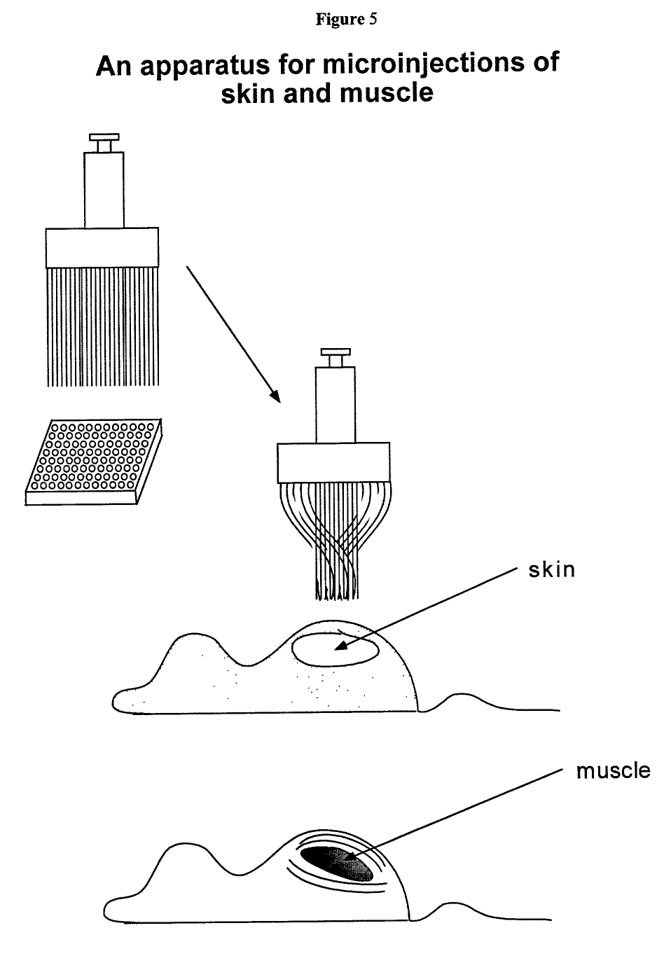

For screening a library of genetic vaccine vectors for ability to transfect cells upon injection into skin or muscle, the invention provides an apparatus which permits large numbers of vectors to be screened efficiently. This apparatus (FIG. 5) is based on 96-well format and is designed to transfer small volumes (2-5 μI) from a microtiter plate to skin or muscle of laboratory animals, such as mice and rats. Moreover, human muscle or skin transplanted into immunodeficient mice can be injected.

The apparatus is designed in such a way that the tips move to fit a microtiter plate. After the reagent of interest has been obtained from the plate, the distance of the tips from each other is decreased to 2-3 mm, enabling transfer of 96 reagents to an area of 1.6 cm× 2.4 cm to 2.4 cm×3.6 cm. The volume of each sample transferred is electronically controlled. Each reagent is mixed with a marker agent or dye to enable recognition of injection site in the tissue. For example, gold particles of different sizes and shapes are mixed with the reagent of interest, and microscopy and immunohistochemistry can be used to identify each injection site and to study the reaction induced by each reagent. When muscle tissue is injected the injection site is first revealed by surgery.

This apparatus can be used to study the effects of large numbers of agents in vivo. For example, this apparatus can be used to screen efficiency of large numbers of different DNA vaccine vectors to transfect human skin or muscle cells transplanted into immunodeficient mice.

V. Optimization of Genetic Vaccine Components

Many factors can influence the efficacy of a genetic vaccine in modulating an immune response. The ability of the vector to enter a cell, for example, has a significant effect on the ability of the vector to modulate an immune response. The strength of an immune response is also mediated by the immunogenicity of an antigen expressed by a genetic vaccine vector and the level at which the antigen is expressed. The presence or absence of costimulatory molecules produced by the genetic vaccine vector can affect not only the strength, but also the type of immune response that arises due to introduction of the vector into a mammal. An increase in the persistence of a vector in an organism can lengthen the time of immunomodulation, and also makes feasible self-boosting vectors which do not require multiple administrations to achieve long-lasting protection. The present invention provides methods for optimizing many of these properties, thus resulting in genetic vaccine vectors that exhibit improved ability to elicit the desired effect on a mammalian immune system.

Genetic vaccines can contain a variety of functional components, whose preferred sequences are best determined by DNA shuffling, the empirical sequence evolution described in detail herein. The methods of the invention involve, in general, constructing a separate library for each of the major vector components by DNA shuffling of multiple homologous starting sequences, or other methods of generating a population of recombinants, resulting in a complex mixture of chimeric sequences. The best sequences are selected from these libraries using the high-throughput assays described below. After one or more cycles of selection from each of the single module libraries, the pools of the best sequences of different modules can be combined by shuffling as long as the screens are compatible. The screens for promoter, enhancer, intron, transfer sequences, mammalian ori, bacterial ori and bacterial marker, and the like, can eventually be combined, resulting in co-optimization of the context of each sequence. An important aspect in these experiments is the selection from large libraries using recursive cycles of shuffling to maximally access all the fortuitous but complex mechanisms that cannot be approached rationally, such as DNA transfer into the cell.

A library of different vectors can be generated by assembling vector modules that provide promoters, cytokines, cytokine antagonists, chemokines, immunostimulatory sequences, and costimulatory molecules using assembly PCR and combinatorial molecular biology. Assembly PCR is a method for assembly of long DNA sequences, such as genes and fragments of plasmids. In contrast to PCR, there is no distinction between primers and template, because the fragments to be assembled prime each other. The library of vector modules obtained as described herein can be fused with promoters, which can themselves be optimized by the DNA shuffling methods of the invention. The resulting genes can be assembled combinatorially into DNA vaccine vectors, where each gene is expressed under a different promoter (e.g., a promoter derived from a library of shuffled CMV promoters), and the vector library is screened as described herein to identify vectors which exhibit the desired effect on the immune system.

The methods of the invention are useful for obtaining genetic vaccines that are optimized for one or more of many properties that influence the efficacy or desirability of the vaccine. These properties include, but are not limited to, the following.

A. Episomal vector maintenance

One property that one can optimize using the sequence recombination methods of the invention is the ability of a genetic vaccine vector to replicate episomally in a mammalian cell. Episomal replication of a vaccine vector is advantageous in many situations. For example, episomally replicating vectors are maintained in a cell for a longer period of time than non-replicating vectors, thus resulting in an increased length of immune response modulation or increased delivery of a therapeutically useful protein. Episomal replication also permits the development of self-boosting vaccines which, unlike traditional vaccines, do not require multiple vaccine administrations. For example, a self-boosting vaccine vector can include an antigen-encoding gene which is under the control of an inducible control element which allows induction of antigen expression, and the corresponding immune response, in response to a specific stimulus. However, screening for naturally occurring vector modules which result in enhanced episomal maintenance using traditional approaches or attempts to rationally design mutants with improved properties would require many person-years of research. The invention provides methods for generating and screening orders of magnitude more diversity in a short time period.

The ability of a genetic vaccine vector to replicate episomally can be optimized by using DNA shuffling to recombine at least two forms of a nucleic acid which is capable of conferring upon a genetic vector the ability to replicate autonomously in mammalian cells. The two or more forms of the episomal replication vector module differ from each other in two or more nucleotides. A library of recombinant episomal replication vector modules is produced, and the library is screened to identify one or more optimized replication vector modules which, when placed in a genetic vaccine vector, confer upon the vector an enhanced ability to replicate autonomously compared to a vector which contains a non-optimized episomal replication vector module.

In one embodiment, the DNA shuffling process is repeated at least once using as a substrate an optimized episomal replication vector module obtained from a previous round of DNA shuffling. The optimized vector module obtained in the earlier round is recombined with a further form of the vector module, which can be the same as one of the forms used in the earlier round, or can be a different form of a nucleic acid that functions as an episomal replication element. Again, a library of recombinant episomal replication vector modules is produced, and the screening process is repeated to identify those episomal replication modules which exhibit enhanced ability to confer episomal maintenance upon a vector containing the module.

Nucleic acids which are useful as substrates for the use of DNA shuffling to optimize episomal replication ability include any nucleic acid that is involved in conferring upon a vector the ability to replicate autonomously in eukaryotic cells. For example, papillomavirus sequences E1 and E2, simian virus 40 (SV40) origin of replication, and the like.

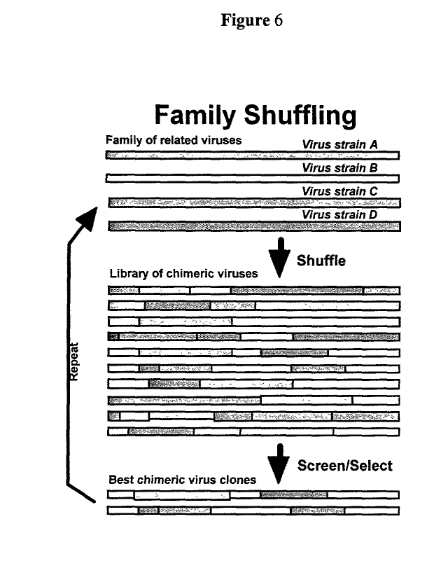

Exemplary episomal replication vector modules that can be optimized using the methods of the invention are genes from human papillomaviruses (HPV) which are involved in episomal replication. HPV are non-tumorigenic viruses which replicate episomally in skin and are stably expressed in vivo for years. Bernard and Apt (1994) Arch. Dermatol. 130: 210. Despite these in vivo properties, it has not been possible to maintain HPV episomally in tissue culture due to underreplication. The invention provides methods by which HPV genes involved in episomal maintenance can be optimized for use in genetic vaccine vectors. HPV genes involved in episomal replication include, for example, the E1 and E2 genes. Thus, according to one embodiment of the invention, either or both of the HPV E1 and E2 genes are subjected to DNA shuffling to obtain a recombinant episomal replication module which, when placed in a nucleic acid vaccine vector, results in increased maintenance of the vector in mammalian cells. In a preferred embodiment, the HPV E1 and E2 genes from different, but closely related, benign HPVs are used in a “family shuffling” procedure, as shown in FIG. 6. For example, family shuffling of HPV E1 and E2 genes from closely related strains of HPV (such as, for example, HPV 2, 27, and 57) can be used to obtain a library of recombinant E1 and E2 genes which are then subjected to an appropriate screening method to identify those that exhibit improved episomal maintenance properties.

To identify recombinant episomal replication vector modules that exhibit improved ability to mediate episomal maintenance, members of the library of recombinant vector modules are inserted into vectors which are introduced into mammalian cells. The cells are propagated for at least several generations, after which cells that have maintained the vector are identified. Identification can be accomplished, for example, employing a vector that includes a selectable marker. Cells containing the library members are propagated in the absence of selection for the selectable marker for at least several generations, after which selective pressure is added. Cells which survive selection are enriched for cells that harbor vectors which contain a recombinant vector module which enhances the ability of the vector to replicate episomally. DNA is recovered from the selected cells and introduced into bacterial host cells, allowing recovery of episomal, non-integrated vectors.

In another embodiment of the invention, the screening step is accomplished by introducing members of the library of recombinant episomal replication vector modules into a vector that includes a polynucleotide that encodes an antigen which, when expressed, is present on the surface of a cell. The library of vectors is introduced into mammalian cells which are propagated for at least several generations, after which cells which display the cell surface antigen on the surface of the cell are identified. Such cells most likely harbor a genetic vaccine vector which enhances the ability of the vector to replicate autonomously. Upon identifying cells which contain an episomally maintained vector, the optimized recombinant episomal replication vector module is obtained and used to construct genetic vaccine vectors. Cell surface antigens which are suitable for use in the screening methods are described above, and others are known to those of skill in the art. Preferably, an antigen is used for which a convenient means of detection is available.

Cells which are suitable for use in the screening methods include both cultured mammalian cells and cells which are present in an animal. To screen for recombinant vector modules that are intended for use in humans, the preferred cells for screening purposes are human cells. Generally, initial screening is accomplished in cell culture, where processing of large libraries of shuffled material is feasible. In a preferred embodiment, cells which display a vector-encoded cell surface antigen on the cell surface are identified by flow cytometry based cell sorting methods, such as fluorescence activated cell sorting. This approach allows very large numbers (>10⁷) cells to be evaluated in a single vial experiment.

Constructs which replicate autonomously in cell culture and give rise to strong marker gene expression can be further tested for durability in vivo in an animal model. For example, mouse models for studies of human tissues in mice in vivo are described in copending U.S. patent application Ser. No. 08/958,822, which was filed on Oct. 28, 1997. Live pieces of human skin are xenotransplanted onto the back of SCID mice, allowing screening of the vector libraries for optimal properties in human cells in vivo. Recursive selection of episomal vectors will provide strong selection pressure for vectors that remain episomal, yet provide high level of gene expression.

In another embodiment, the screening step involves introducing a genetic vaccine vector which includes the recombinant episomal replication vector module, as well as polynucleotide that encodes an antigen or pharmaceutically useful protein, into a mammal that has a functional human immune system. The animal is then tested for the existence of an immune response against the antigen. In a preferred embodiment, the mammals used for such assays are non-human mammals that have a functional human immune system. For example, a functional human immune system can be created in an immunodeficient mouse by introducing one or more of a human fetal tissue selected from the group consisting of liver, thymus, and bone marrow (Roncarolo et al. (1996) Semin. Immunol. 8: 207).

Stable episomal vectors which are obtained using the methods of the invention are useful not only as genetic vaccines, but also are useful tools in other library screening applications. In contrast to randomly integrating and transient vectors, episomally maintained vectors result in high signal-to-noise ratios upon FACS selection, and they also significantly improve the possibility to recover the plasmids from a small number of selected cells.

B. Evolution of Optimized Promoters for Expression of an Antigen

In another embodiment, the invention provides methods of optimizing vector modules such as promoters and other gene expression control signals. Usually, a coding sequence for an antigen that is delivered by a genetic vaccine is operably linked to an additional sequence, such as a regulatory sequence, to ensure its expression. These regulatory sequences can include one or more of the following: an enhancer, a promoter, a signal peptide sequence, an intron and/or a polyadenylation sequence. A desirable goal is to increase the level of expression of functional expression product relative to that achieved with conventional vectors. The efficacy of a genetic vaccine vector often depends on the level of expression of an antigen by the vaccine vector. An optimized promoter and/or other control sequence is likely to result in improved efficacy of genetic vaccinations, reduce the amount of DNA required for protective immunity and thereby the cost of vaccination. Moreover, it is sometimes desirable to have control over the type of cell in which a gene is expressed, and/or the timing of antigen expression. The methods of the invention provide for optimization of these and other factors which are influenced by promoters and other control sequences.

Improved expression of selection markers can be achieved by performing DNA shuffling, for example. Expression can effectively be improved by a variety of means, including increasing the rate of production of an expression product, decreasing the rate of degradation of the expression product or improving the capacity of the expression product to perform its intended function. The methods involve subjecting to DNA shuffling polynucleotides which are involved in control of gene expression. At least first and second forms of a nucleic acid that comprises a control sequence, which forms differ from each other in two or more nucleotides, are recombined as described above. The resulting library of recombinant transfer modules are screened to identify at least one optimized recombinant control sequence that exhibits enhanced strength, inducibility, or specificity.

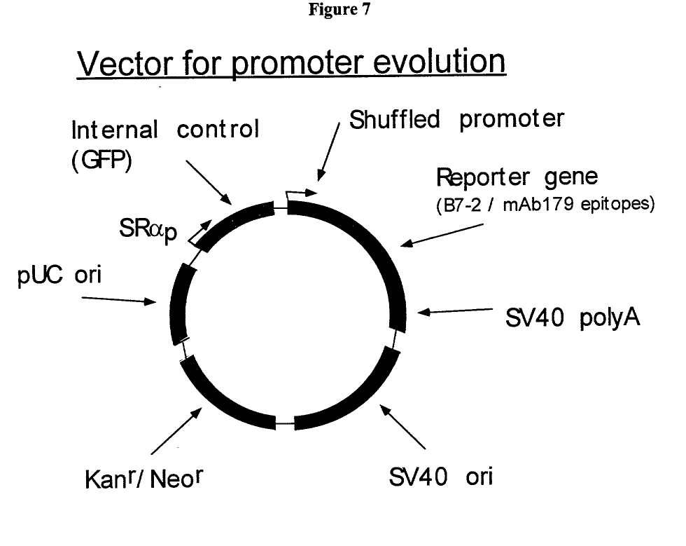

The substrates for recombination can be the full-length vectors, or fragments thereof, which include a coding sequence and/or regulatory sequences to which the coding sequence is operably linked. The substrates can include variants of any of the regulatory and/or coding sequence(s) present in the vector. If recombination is effected at the level of fragments, the recombinant segments should be reinserted into vectors before screening. If recombination proceeds in vitro, vectors containing the recombinant segments are usually introduced into cells before screening. An example of a vector suitable for use in screening of shuffled promoters and other regulatory regions is shown in FIG. 7.

Cells containing the recombinant segments can be screened by detecting expression of the gene encoded by the selection marker. For purposes of selection and/or screening, a gene product expressed from a vector is sometimes an easily detected marker rather than a product having an actual therapeutic purpose, e.g., a green fluorescent protein (see, Crameri (1996) Nature Biotechnol. 14: 315-319) or a cell surface protein. For example, if this marker is green fluorescent protein, cells with the highest expression levels can be identified by flow cytometry-based cell sorting. If the marker is a cell surface protein, the cells are stained with a reagent having affinity for the protein, such as antibody, and again analyzed by flow cytometry-based cell sorting. However, some genes having a therapeutic purpose, e.g., drug resistance genes, themselves provide a selectable marker, and no additional or substitute marker is required. Alternatively, the gene product can be a fusion protein comprising any combination of detection and selection markers. Internal reference marker genes can be included on the vector to detect and compensate for variations in copy number or insertion site.

Recombinant segments from the cells showing highest expression of the marker gene can be used as some or all of the substrates in a further round of recombination and screening, if additional improvement is desired.

1. Constitutive promoters

The invention provides methods of evolving nucleotide sequences that are capable of directing constitutive expression of a gene of interest which is operably linked to the control sequence. Typically, the control sequences, which can include promoters, enhancers, and the like, are evolved so that a gene of interest is expressed at a higher level than is a gene operably linked to a non-evolved control sequence. To screen for control sequences which are of increased strength, a recombinant library of control sequences can be introduced into a population of cells and the level of expression of a detectable marker operably linked to the control sequences determined. Preferably, the optimized promoter is capable of expressing an operably linked gene at a level that is at least about 30% greater than that of a control promoter construct, more preferably the optimized promoter is at least about 50% stronger than a control, and most preferably at least about 75% or more stronger than a control promoter.

Examples of promoters which can be used as substrates in the methods include any constitutive promoter that functions in the intended host cell. The major immediate-early (IE) region transcriptional regulatory elements, including promoter and enhancer sequences (the promoter/enhancer region), of cytomegalovirus (CMV) is widely used for regulating transcription in vectors used for gene therapy because it is highly active in a broad range of cell types. Optimized CMV transcriptional regulatory elements which direct increased levels of antigen expression is generated by the recursive recombination methods of the invention, resulting in improved efficacy of gene therapy. As the CMV promoter and enhancer is active in human and animal cells, the improved CMV promoter/enhancer elements are used to express foreign genes both in animal models and in clinical applications. Other constitutive promoters that are amenable to use in the claimed methods include, for example, promoters from SV40 and SRα, and other promoters known to those of skill in the art.

In a preferred embodiment, a library of chimeric transcriptional regulatory elements is created by DNA shuffling of wild-type sequences from two or more of the five related strains of CMV. The promoter, enhancer and first intron sequences of the IE region are obtained by PCR from the CMV strains: human VR-538 strain AD169 (Rowe (1956) Proc. Soc. Exp. Biol. Med. 92:418; human V-977 strain Towne (Plotkin (1975) Infect. Immunol. 12:521-527); rhesus VR-677 strain 68-1 (Asher (1969) Bacteriol. Proc. 269:91); vervet VR-706 strain CSG (Black (1963) Proc. Soc. Exp. Biol. Med. 112:601); and, squirrel monkey VR-1398 strain SqSHV (Rangan (1980) Lab. Animal Sci. 30:532). The promoter/enhancer sequences of the human CMV strains are 95% homologous, and share 70% homology with the sequences of the monkey isolates, allowing the use of family shuffling to generate a library great diversity. Following shuffling, the library is cloned into a plasmid backbone and used to direct transcription of a marker gene in mammalian cells. An internal marker under the control of a native promoter is typically included in the plasmid vector, which will allow analysis and sorting of cells harboring equal numbers of vectors. Expression markers, such as green fluorescent protein (GFP) and CD86 (also known as B7.2, see Freeman (1993) J. Exp. Med. 178:2185, Chen (1994) J. Immunol. 152:4929) can also be used. In addition, transfection of SV40 T antigen-transformed cells can be used to amplify a vector which contains an SV40 origin of replication. The transfected cells are screened by FACS sorting to identify those which express high levels of the marker gene, normalized against the internal marker to account for differences in vector copy numbers per cell. If desired, vectors carrying optimal, recursively recombined promoter sequences are recovered and subjected to further cycles of shuffling and selection.

2. Cell-specific promoters

One of the safety concerns associated with genetic vaccines has been the possibility of autoimmune disorders following introduction of foreign antigens into host cells. This risk can be reduced if the pathogen antigen is specifically expressed in professional APCs that express the proper costimulatory molecules. Although it is somewhat debatable which cells are the most important cells expressing the pathogen antigen following genetic vaccinations, it is likely that professional APCs are involved. It has been shown that blood monocytes express antigen following intramuscular injection of genetic vaccine vectors, and dendritic cells derived from lymph nodes of vaccinated animals efficiently induced antigen-specific T cell activation (C. Bona, The First Gordon Conference on Genetic Vaccines, Plymouth, N.H., Jul. 21, 1997). These data, together with previous studies indicating that small number of dendritic cells expressing antigen or antigenic peptides is sufficient to induce activation of antigen-specific T cells (Thomas and Lipsky, Stem Cells 14:196, 1996), support the conclusion that genetic vaccines specifically expressed in professional APC, such as dendritic cells and macrophages, are likely to provide efficient induction of protective immunity with minimized chance of adverse effects.

The present invention provides methods of obtaining promoters and enhancers that induce high expression levels specifically in professional APCs. Previously existing APC-specific vectors did not provide sufficient expression levels following genetic vaccinations. The methods involve performing DNA shuffling as described above using as substrates different forms of a nucleic acid that comprises an APC-specific promoter or other control signal. Suitable promoters include, for example, the MHC Class II, and the CD11b, CD11c, and CD40 promoters. Natural diversity of the promoters can be exploited as a highly appropriate source of substrates for the DNA shuffling. For example, genomic DNA from monkeys, pigs, dogs, cows, cats, rabbits, rats and mice, can be obtained, and the proper sequences obtained by using multiple PCR primers specific for the most conserved regions based on known sequence information. The selection of the optimal promoters can be done in monocytic or B cell lines, such as U937, HL60 or Jijoye, using FACS-sorting. In addition, SV40⁺cell lines, such as COS-1 and COS-7, can be used to improve the recovery of the plasmids. Further analysis can be undertaken in human dendritic cells obtained by culturing peripheral blood monocytes in the presence of IL-4 and GM-CSF as described (Chapuis et al. (1997) Eur. J. Immunol. 27: 431).

3. Inducible promoters

A particularly desirable property of a genetic vaccines would be an ability to induce the promoter controlling transgene expression simply by taking an innocuous oral drug, resulting in a boost of the immune response. Essential requirements for inducible promoters are low base-line expression and strong inducibility. Several promoters with exquisite in vitro regulation exist, but the expression level and inducibility of each is too low to be useable in vivo. The invention overcome these problems by DNA shuffling using as substrates two or more variants of a nucleic acid that functions as an inducible control sequence. Suitable substrates include, for example, tetracycline and hormone inducible expression systems, and the like. Hormones that have been used to regulate gene expression include, for example, estrogen, tomoxifen, toremifen and ecdysone (Ramkumar and Adler (1995) Endocrinology 136: 536-542). Libraries of recombinant inducible promoters are screened as described above in the presence and absence of the inducer.

The most commonly used inducible gene expression protocol is the tetracycline responsive system, which provides possibilities to both induce and turn off gene expression (Gossen and Bujard (1992) Proc. Nat'l. Acad. Sci. USA 89: 5547; Gossen et al. (1995) Science 268: 1766). A repressor gene is located on the plasmid and binds to an operator in the promoter. Tetracycline or doxycycline modulates the binding ability of the repressor. Interestingly, four amino acid changes convert the repressor into an activator. In addition to the tetracycline responsive system, other candidates for inducible promoter evolution include the ecdysone responsive element (No et al., Proc. Nat'l. Acad. Sci. USA 93: 3346, 1997).

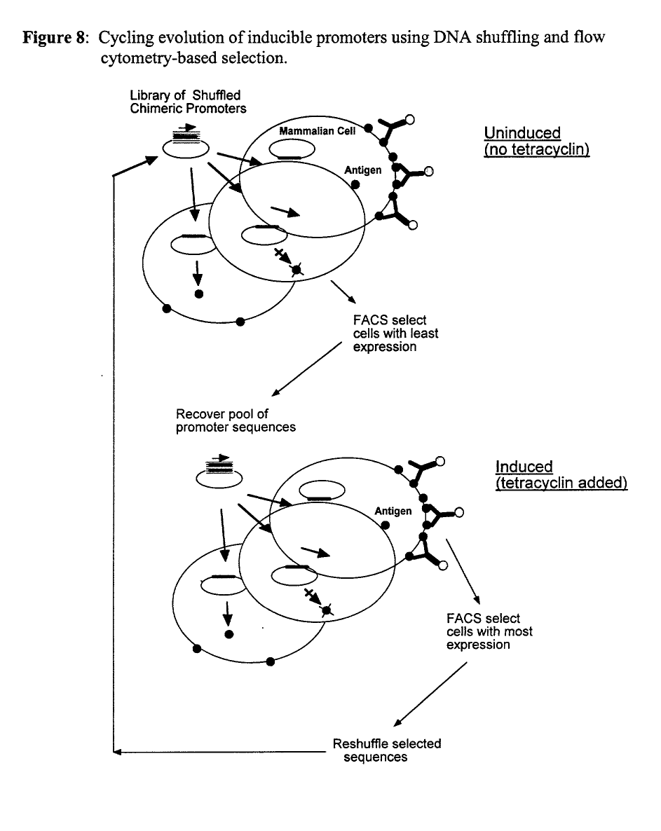

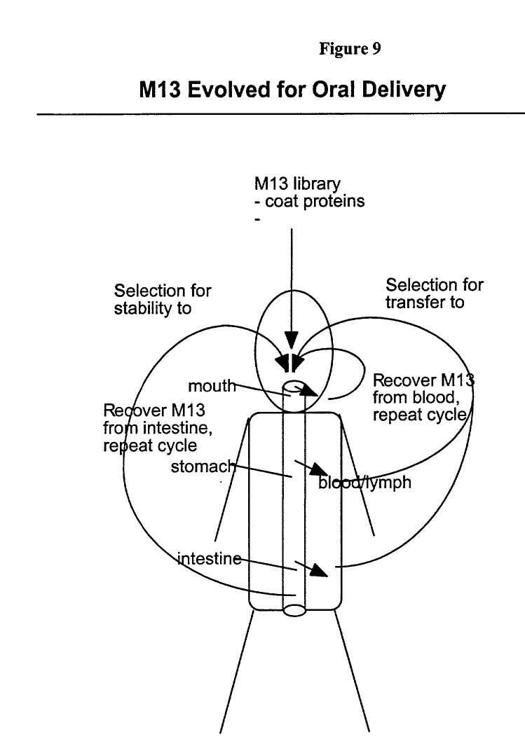

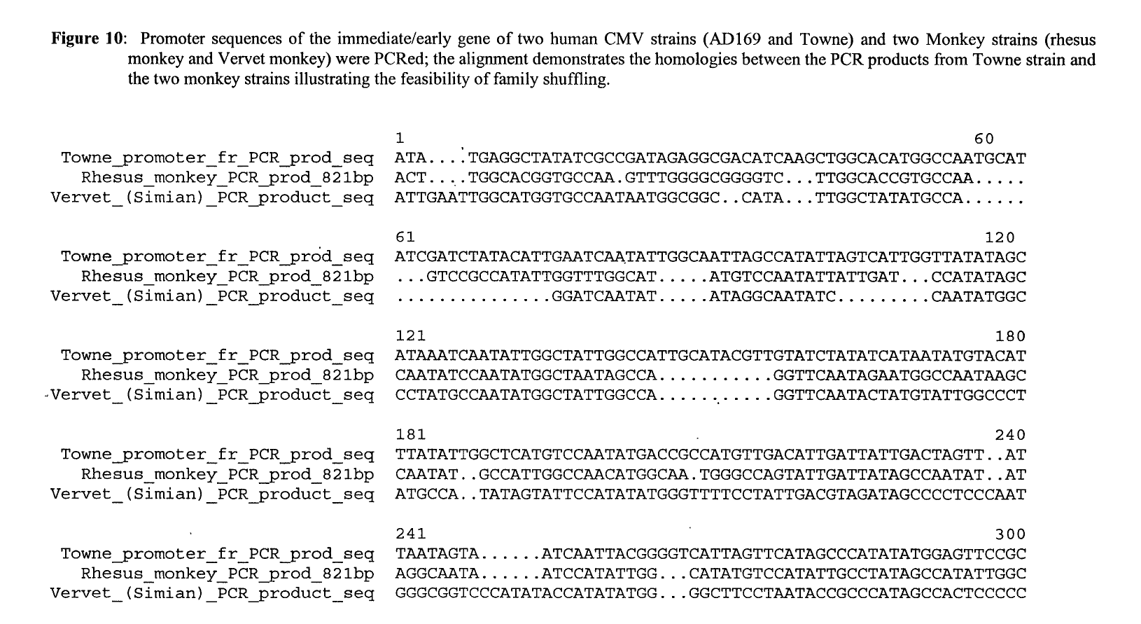

Inducible promoters such as those obtained using the methods of the invention are useful in autoboost vaccines. Particularly when combined with a stably maintained episomal vector obtained as described above, the inducible promoters provide a means by which a vaccine dose can be administered subsequent to the initial administration simply by ingestion of a reagent that causes induction of the inducible promoter. FIG. 8 demonstrates a flow cytometry-based screening protocol that is suitable for optimization of inducible promoters.

The functionality of autoboosting vaccines can be tested in a mouse model such as that described above. Genetic vaccine vectors are injected into the skin of normal mice and into human skin in SCID-human skin mice. A gene encoding hepatitis B surface antigen (HBsAg) or other surface antigen is incorporated into these vectors enabling direct measurements of the levels of antigen produced, because HBsAg levels can be measured in cell culture supemates and in the circulation of the mice. The drug inducing the expression of the antigen is given after 1, 2, 4 and 6 weeks, and the expression levels of HBsAg are studied. Moreover, the levels of anti-HBsAg antibodies are measured. The mice are also injected with a vector containing a pathogen antigen discovered by ELI, and specific immune responses are followed.

Combining the SCID-human skin model with traditional SCID-hu mouse model (Roncarolo et al., Semin. Immunol. 8: 207, 1996) allows the assessment of functionality of autoboosting genetic vaccines in human immune system in vivo, and also allows measurements of human Ab responses in vivo. This model can also be used to assess production of HBsAg after oral boosting of novel genetic vaccine vectors harboring the gene encoding HBsAg.

C. Evolution of Genetic Vaccine Vectors for Increased Vaccination Efficacy and Ease of Vaccination

This section discusses the application of the invention to some specific goals in genetic vaccination. Many of these goals relate to improvements in vectors used in vaccine delivery. Unless otherwise indicated the methods are applicable to both viral and nonviral vectors.

1. Topical application of genetic vaccine vectors

The invention provides methods of improving the ability of genetic vaccine vectors to induce a desired response after topical application of the vector. Adenoviral vectors topically applied to bare skin have been shown to be capable of acting as vaccine antigen delivery vehicles (Tang et al. (1997) Nature 388: 729-730). An adenoviral vector that encoded carcinoembryonic antigen (CA) was shown to induce antibodies specific for CA after application to the skin. However, the efficiency of topical application is generally quite low, and protective immune responses have not been demonstrated after topical application.

The invention provides methods of obtaining vectors that exhibit improved efficiency when topically administered. Several factors can influence topical application efficiency, each of which can be optimized using the methods of the invention. For example, the invention provides methods of improving vector affinity for skin cells, improved skin cell transfection efficiency, improved persistence of the vector in skin cells (both through improved replication or through avoidance of destruction by immune cells), and improved antigen expression in skin cells, and improved induction of an immune response.

These methods involve performing DNA shuffling using as substrates plasmid, naked DNA vectors, or viral vector nucleic acids, including, for example, adenoviral vectors. Libraries of shuffled nucleic acids are screened to identify those nucleic acids that confer upon a vector an enhanced ability to induce an immune response upon topical administration. Screening can be conducted by, for example, topically applying a library of shuffled vectors to skin, either mouse skin, monkey skin, or human skin that has been transplanted to immunodeficient mice, or to normal human skin in vivo. Vectors that persist and/or provide efficient and long-lasting expression of marker gene are recovered from the skin samples. In a preferred embodiment, the desired cells are first selected by cell sorting, magnetic beads, or panning. For example, recovery can be effected through expression of a marker gene (e.g., GFP) and detecting cells that are transfected using fluorescence microscopy or flow cytometry. Cells that express the marker gene can be isolated using flow cytometry based cell sorting. Screening can also involve selection of vectors that induce the highest specific antibody or CTL responses upon administration to a test mammal, or the identification of vectors that provide an enhanced protective immune response to challenge with a corresponding pathogen. Shuffled polynucleotides are then recovered, e.g., by polymerase chain reaction, or the entire vectors can be purified from these selected cells. If desired, further optimization of topical application efficiency can be obtained by subjecting the recovered shuffled polynucleotides to new rounds of shuffling and selection.

Genetic vaccine vectors that are optimized for topical application can be applied topically to the skin, or by intramuscular, intravenous, intradermal, oral, anal, or vaginal delivery. The vector can be delivered in any of the suitable forms that are known to those of skill in the art, such as a patch, a cream, as naked DNA, or as a mixture of DNA and one or more transfection-enhancing agents such as liposomes and/or lipids. In preferred embodiments, the genetic vaccine vector is applied after the skin or other target is rendered more susceptible to uptake of the vector by, for example, mechanical abrasion, removal of hair (e.g., by treatment with a commercially available product such as Nair™, Neet™, and the like). In one embodiment, the skin is pretreated with proteases or lipases to make it more susceptible to DNA delivery. In addition, the DNA can be mixed with the proteases or lipases to enhance gene transfer. Alternatively, a droplet containing the vector and other vaccine components, if any, can simply be administered to the skin.

2. Enhanced ability to escape host immune system

Immunogenicity is a particular concern with viral vectors, since a host immune response can prevent a virus from reaching its intended target particularly in repeated administrations. The efficacy of some viral vectors which are used for genetic vaccination and gene delivery is limited by host immune responses directed against the viral vector. For example, most individuals have pre-existing antibodies against adenovirus. Adenoviral vectors can sometimes induce strong immune responses which can destroy cells harboring adenoviral vectors or clear adenoviral vectors from the host even before target cells are entered. Cellular immune responses can also be induced against nonviral vectors administered in naked form or shielded with a coat such as liposomes.

The invention provides methods to create genetic vaccine vectors that can escape immune responses that would otherwise be detrimental to obtaining the desired effect. These methods are useful for prolonging expression and secretion of pathogen antigen or pharmaceutically useful protein by genetic vaccine vectors. Several strategies are provided by which one can improve a genetic vaccine vector's ability to avoid the humoral (Ab) and cellular (CTL) immune systems. These strategies can be used in combination to obtain optimal avoidance such as may be required for highly immunogenic vectors such as adenovirus.

In one embodiment, the invention provides methods of obtaining viral vectors that are capable of escaping a host CTL immune response. This method can be used in conjunction with methods for obtaining genetic vaccine vectors that can escape the humoral response; the combination of approaches is often desirable, as different viral serotypes often have CTL epitopes in common, suggesting that virus variants which are not recognized by antibodies still are likely to be recognized by CTLs. This embodiment of the invention involves incorporating into genetic vaccines one or more components that inhibit peptide transport and/or MHC class I expression. An essential element in the activation of cytotoxic T lymphocyte (CTL) responses is an interaction between T cell receptors on CTLs and antigenic peptide-MHC class I molecule complexes on antigen presenting cells. Expression of MHC class I molecules on thymocytes and antigen presenting cells is a requirement for maturation and activation of antigen-specific CD8³⁰ T lymphocytes. Thus, genes that encode inhibitors of MHC class I-mediated antigen presentation can be shuffled as described herein and placed into viral vectors to obtain vectors that, when present in target cells, do not induce destruction of the target cells by the cells of the immune system. This can result in prolonged survival of cells harboring genetic vaccine vectors, including those that express a pathogen antigen, as well as vectors that express a pharmaceutically useful protein. In the case of genetic vaccines, reduced expression of MHC class I molecules will allow secretion of the pathogen antigen, which then will be presented by professional antigen presenting cells elsewhere. In the case of vectors encoding pharmaceutical proteins, reduced expression of MHC class I molecules prevents recognition by the immune system prolonging the survival of the cells expressing the gene.

Among the proteins involved in MHC class I molecule expression and antigen presentation are those encoded by TAP genes (transporters associated with antigen processing), which are described above. In one embodiment of the invention, genes that encode inhibitors of TAP activity are shuffled to obtain genes that encode optimized TAP inhibitors. The substrates for these methods can include, for example, one or more of the viral genes that are known to regulate levels of MHC class I molecule expression. TAP1 and TAP2 gene expression is 5-10-fold and 100-fold reduced, respectively, in cells transformed by adenovirus 12, which results in reduced class I expression and thus leads to reduced virus-specific cytotoxic T lymphocyte responses. Similarly, TAP gene expression is downregulated in 49% of HPV-16⁺cervical carcinomas (Seliger et al. (1997) Immunol. Today 18: 292). Thus, adenovirus and HPV viral nucleic acids provide examples of suitable substrates for carrying out the methods of the invention. Additional examples of suitable DNA shuffling substrates for this embodiment of the invention include the human cytomegalovirus (CMV) encoded genes US2, US3 and US11, which can downregulate MHC class I expression (Wiertz et al. (1996) Nature 384: 432 and Cell (1996) 84: 769; Ahn et al. (1996) Proc. Nat'l. Acad. Sci. USA 93: 10990). Another human CMV gene that encodes an inhibitor of TAP-dependent peptide translocation is US6 (Lehner et al. (1997) Proc. Nat'l. Acad. Sci. USA 94: 6904-9). Cells transfected with US6 had reduced expression of MHC class I molecules on their surface and reduced capacity to activate cytotoxic T lymphocytes. Thus, in one embodiment, the invention involves DNA shuffling of this cluster of genes (approximately 7 kb), or fragments thereof, in order to identify the sequences that are most potent in inhibiting the expression of MHC class I molecules. Such optimized TAP inhibitor polynucleotide sequences are useful not only for use in constructing vectors that can escape CTL immune responses, but also for generation of animal models for use with human viruses that normally are eliminated in laboratory animals due to their immunogenicity. The desired expression levels and functional properties of TAP inhibitors may vary depending on whether genetic vaccine vector, gene therapy vector or animal model is evolved.

Alternative embodiments of the invention involve DNA shuffling of other genes that are involved in downregulating expression of MHC class I molecules and/or antigen presentation. Examples of other possible target genes include genes encoding adenoviral E3 protein, herpes simplex ICP47 protein, and tapasin antagonists (Seliger et al. (1997) Immunol. Today 18:292-299; Galoncha et al. (1997) J. Exp. Med. 185: 1565-1572; Li et al. (1997) Proc. Nat'l. Acad. Sci. USA 94: 8708-8713; Ortmann et al. (1997) Science 277: 1306-1309.

Because reduced expression of MHC class I molecules on cell surfaces may act as a stimulus for NK cells, it may be useful to include in genetic vaccine vectors a gene that encodes an MHC like molecule that inhibits NK cell function but is unable to present antigens to T lymphocytes. An example of such molecule is MHC class I homologue encoded by cytomegalovirus (Farrell et al. (1997) Nature 386: 510-514).

The invention also provides methods of obtaining viral vectors that exhibit an enhanced capability of avoiding attack by CD4⁺T lymphocytes. Such vectors are particularly useful in situations where the target cells are capable of expressing MHC class II molecules, such as in the case of vaccinations and gene therapy targeted to the cells of the immune system. Substrates for DNA shuffling include genes that encode inhibitors of MHC class II molecules such as, for example, IL-10 and antagonists of IFN-γ (such as soluble IFN-γ receptor).

Vectors that have the greatest capability of escaping the host immune system, will typically include DNA sequences that result in inhibition of MHC class I expression and MHC class II expression, and additional sequences that encode homologs of MHC class I molecules. The properties of all these can be further improved by DNA shuffling according to the methods of the invention.

Once a library of shuffled DNA molecules is obtained, any of several methods are available for screening the library to identify those polynucleotides that, when present in a viral vector (or in an animal model) exhibit the desired effect on the host immune response. For example, to obtain shuffled polynucleotides that inhibit MHC class I expression and/or antigen presentation, a library of shuffled genes can be incorporated into genetic vaccine or gene therapy vectors and transfected into human cell lines, such as, for example, HeLa, U937 or Jijoye, in a single tube transfection. Primary human monocytes, or dendritic cells generated by culturing human cord blood cells or monocytes in the presence of IL-4 and GM-CSF, are also suitable. Initial screening can be done using FACS-sorting. Cells expressing the lowest levels of MHC class I molecules are selected, the polynucleotides that encode the MHC inhibitors, or whole plasmids containing the sequences, are recovered. If desired, the selected sequences can be subjected to new rounds of shuffling and selection. Cells expressing the lowest levels of MHC class I molecules are expected to have the lowest capacity to induce CTL responses.

Another screening method involves incorporating libraries of shuffled polynucleotides that encode inhibitors of MHC class I expression are incorporated into human papillomavirus (HPV) vectors. This library is injected into the skin of mice. Normally, murine cells expressing HPV are destroyed by the host immune system. However, cells expressing potent inhibitors of peptide transportation and/or MHC class expression will be able to escape the immune response. The cells that express a marker gene present on the vector, such as GFP, for extended periods of time are selected, the sequences or whole plasmids are recovered, and, if further optimization is desired, the selected sequences are subjected to new rounds of shuffling and selection. Long-lasting maintenance of HPV in mice will allow drug screening and vaccine studies, which to date have not been possible due to high immunogenicity of HPV in mice.

In another embodiment, the libraries of shuffled polynucleotides encoding inhibitors of MHC class I expression are incorporated into human adenovirus vectors. This library is transfected into human cell lines, such as HeLa cells, and cells expressing the lowest levels of MHC class I molecules are selected as described above. The sequences that provide the lowest levels of MHC class I expression are further tested by analyzing the capacity of antigen-presenting cells transfected with adenovirus harboring evolved inhibitors of MHC class I expression to activate specific T cell lines or clones. These inhibitors will block efficient presentation of immunogenic peptides, and hence, will strongly downregulate activation of antigen-specific CTLs allowing long-lasting transgene expression in vivo.

Methods to screen for improved inhibitors of MHC class II expression include detection of MHC class II molecules on the surface of the target cells by fluorescent labeled specific monoclonal antibodies, fluorescence microscopy, and flow cytometry. In addition, the inhibitors can be analyzed in functional assays by studying the capacity of the inhibitors to block activation of MHC class II restricted antigen-specific CD4⁺T lymphocytes. For example, one can determine the capacity of the inhibitor to inhibit induction of CD4⁺T cell proliferation induced by autologous antigen presenting cells, such as monocytes, dendritic cells, B cells or EBV-transformed B cell lines, that harbor genes encoding the MHC class II inhibitor or have been treated with supernatant containing the inhibitor.

3. Enhanced Antiviral Activity

The invention also provides methods of obtaining a recombinant viral vector which has an enhanced ability to induce an antiviral response in a cell. DNA shuffling is used to produce a library of recombinant viral vectors. The library is transfected into a population of mammalian cells, which are then tested for ability to induce an antiviral response. One suitable test involves staining the cells for the presence of Mx protein, which is produced by cells that are exhibiting an antiviral response (see, e.g., Hallimen et al. (1997) Pediatric Research 41: 647-650; Melen et al. (1994) J. Biol. Chem. 269: 2009-2015). Recombinant viral vectors can be isolated from cells which stain positive for Mx protein. These recombinant viral vectors from positive staining cells are enriched for those that exhibit enhanced ability to induce an antiviral response. Viral vectors for which this method is useful include, for example, influenza virus.

4. Evolution of vectors having increased copy number in production cells

The invention provides methods for obtaining vector components that, when present in a genetic vaccine vector (such as a plasmid) the ability to replicate to a high copy number in a cell used to produce the vector. Plasmids can incorporate various heterologous DNA sequences, however the size or the nature of the cloned sequences in a given plasmid vector may render that vector less able to grow to high copy number in the bacteria in which it is propagated. It is therefore desirable to have a method to increase the plasmid copy number after all elements have been cloned into the vector. This is especially important when the plasmid is to be manufactured on a large scale as will be the case for genetic vaccines.

The methods of the invention involve incorporating into the plasmid one or more polynucleotide sequences that bind proteins which would otherwise be toxic to the bacterium. One suitable toxic moiety and binding site combination is the transcription factor GATA-1 and its recognition site. It has been shown that expression of a DNA-binding fragment of GATA-1 is toxic to bacteria; this toxicity apparently results from inhibition of bacterial DNA replication. Trudel et al. ((1996) Biotechniques 20: 684-693) have described a plasmid (PGATA) that expresses the Z2B2 region of GATA-1 as a GST fusion protein. The expression of the fusion protein in this plasmid is under the control of the IPTG-inducible lac promoter. The GST-GATA-1 fragment also binds strongly to a sequence from the mouse β-globin gene promoter as well as to the C-oligonucleotide from the β-globin gene 3′ enhancer; either or both of these are suitable for use as binding sites in the methods of the invention.

The plasmids preferably also include a selectable marker such as, for example, kanamycin resistance (aminoglycoside 3′-phosphotransferase (EC 2.7.1.95)) and the like. The plasmid backbone polynucleotide sequence is subjected to DNA shuffling as described herein to generate a library of plasmids which have different backbone sequences and possibly different supercoil densities. In order to introduce sufficient sequence diversity to search for improved function, it is preferable to perform family DNA shuffling. This can be accomplished in the context of the present invention by including in the shuffling reaction only a single form of the selectable marker. In this way, significant diversity can be achieved in the shuffled library to recover a plasmid which is improved in its growth properties while fully retaining the appropriate selection function of the plasmid.

The selection for high copy number plasmids is performed by introducing the library of shuffled recombinant plasmids into the desired host cell. The host cells also express the toxic moiety, preferably under the control of a promoter which is inducible. For example, the pGATA plasmid is suitable for use in E. coli host cells. The shuffled plasmids are introduced into the cells under non-inducing conditions. Transformed cells are then placed under conditions which induce expression of the toxic moiety. For example, E. coli cells that contain pGATA can be placed on media containing increasing concentrations of IPTG. Those target plasmids which grow to high copy number in the bacteria will express correspondingly higher numbers of the binding sequences for GATA-1. The target plasmids will bind the GST-GATA-1 fusion protein and thus neutralize the toxic effects on the bacteria.

Plasmids with the highest copy number are detected as those which confer the best growth to bacteria on the inducer-containing growth media. Such plasmids can be recovered and transformed into bacteria which lack the gene that encodes the toxic moiety; these plasmids should retain their high copy number characteristics. Further rounds of shuffling can be used to isolate high copy number plasmids by the above selection procedure. Alternatively, manual screening can be done in the bacterial host of choice, lacking the toxic moiety-encoding plasmid, to avoid any effects due to the presence of this extraneous plasmid.

### VI. Genetic Vaccine Pharmaceutical Compositions and Methods of Administration

The vector components and multicomponent genetic vaccines of the invention are useful for treating and/or preventing various diseases and other conditions. For example, genetic vaccines that employ the reagents obtained according to the methods of the invention are useful in both prophylaxis and therapy of infectious diseases, including those caused by any bacterial, fungal, viral, or other pathogens of mammals. The reagents obtained using the invention can also be used for treatment of autoimmune diseases including, for example, rheumatoid arthritis, SLE, diabetes mellitus, myasthenia gravis, reactive arthritis, ankylosing spondylitis, and multiple sclerosis. These and other inflammatory conditions, including IBD, psoriasis, pancreatitis, and various immunodeficiencies, can be treated using genetic vaccines that include vectors and other components obtained using the methods of the invention. Genetic vaccine vectors and other reagents obtained using the methods of the invention can be used to treat allergies and asthma. Moreover, the use of genetic vaccines have great promise for the treatment of cancer and prevention of metastasis. By inducing an immune response against cancerous cells, the body's immune system can be enlisted to reduce or eliminate cancer.

In presently preferred embodiments, the optimized genetic vaccine components are used in conjunction with other optimized genetic vaccine reagents. For example, an antigen that is useful for a particular condition can be optimized by methods analogous to the recombination and screening methods described herein (see, copending, commonly assigned U.S. patent application Ser. No. _____, entitled “Antigen Library Immunization”, which was filed on Feb. 10, 1999 as TTC Attorney Docket No. 18097-028710US). The polynucleotide that encodes the recombinant antigenic polypeptide can be placed under the control of a promoter, e.g., a high activity or tissue-specific promoter. The promoter used to express the antigenic polypeptide can itself be optimized using recombination and selection methods analogous to those described herein. The vector can contain immunostimulatory sequences such as are described in copending, commonly assigned U.S. patent application Ser. No. ______, entitled “Optimization of Immunomodulatory Molecules,” filed as TTC Attorney Docket No. 18097-030300US on Feb. 10, 1999. It is sometimes advantageous to employ a genetic vaccine that is targeted for a particular target cell type (e.g., an antigen presenting cell or an antigen processing cell); suitable targeting methods are described in copending, commonly assigned U.S. patent application Ser. No. _____, entitled “Targeting of Genetic Vaccine Vectors,” filed on Feb. 10, 1999 as TTC Attorney Docket No. 18097-030200US.

Genetic vaccines, including the multicomponent genetic vaccines described herein, can be delivered to a manmmal (including humans) to induce a therapeutic or prophylactic immune response. Vaccine delivery vehicles can be delivered in vivo by administration to an individual patient, typically by systemic administration (e.g., intravenous, intraperitoneal, intramuscular, subdermal, intracranial, anal, vaginal, oral, buccal route or they can be inhaled) or they can be administered by topical application. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

A large number of delivery methods are well known to those of skill in the art. Such methods include, for example liposome-based gene delivery (Debs and Zhu (1993) WO 93/24640; Mannino and Gould-Fogerite (1988) BioTechniques 6(7): 682-691; Rose U.S. Pat No. 5,279,833; Brigham (1991) WO 91/06309; and Felgner et al. (1987) Proc. Natl. Acad. Sci. USA 84: 7413-7414), as well as use of viral vectors (e.g., adenoviral (see, e.g., Bems et al. (1995) Ann. NY Acad. Sci. 772: 95-104; Ali et al. (1994) Gene Ther. 1: 367-384; and Haddada et al. (1995) Curr. Top. Microbiol. Immunol. 199 (Pt 3): 297-306 for review), papillomaviral, retroviral (see, e.g., Buchscher et al. (1992) J. Virol. 66(5) 2731-2739; Johann et al. (1992) J. Virol 66 (5):1635-1640 (1992); Sommerfelt et al., (1990) Virol. 176:58-59; Wilson et al. (1989) J. Virol. 63:2374-2378; Miller et al., J. Virol. 65:2220-2224 (1991); Wong-Staal et al., PCT/US94/05700, and Rosenburg and Fauci (1993) in Fundamental Immunology, Third Edition Paul (ed) Raven Press, Ltd., New York and the references therein, and Yu et al., Gene Therapy (1994) supra.), and adeno-associated viral vectors (see, West et al. (1987) Virology 160:38-47; Carter et al. (1989) U.S. Pat. No. 4,797,368; Carter et al. WO 93/24641 (1993); Kotin (1994) Human Gene Therapy 5:793-801; Muzyczka (1994) J. Clin. Invst. 94:1351 and Samulski (supra) for an overview of AAV vectors; see also, Lebkowski, U.S. Pat. No. 5,173,414; Tratschin et al. (1985) Mol. Cell. Biol. 5(11):3251-3260; Tratschin, et al. (1984) Mol. Cell. Biol., 4:2072-2081; Hermonat and Muzyczka (1984) Proc. Natl. Acad. Sci. USA, 81:6466-6470; McLaughlin et al. (1988) and Samulski et al. (1989) J. Virol., 63:03822-3828), and the like.

“Naked” DNA and/or RNA that comprises a genetic vaccine can be introduced directly into a tissue, such as muscle. See, e.g., U.S. Pat. No. 5,580,859. Other methods such as “biolistic” or particle-mediated transformation (see, e.g., Sanford et al., U.S. Pat. No. 4,945,050; U.S. Pat. No. 5,036,006) are also suitable for introduction of genetic vaccines into cells of a mammal according to the invention. These methods are useful not only for in vivo introduction of DNA into a mammal, but also for ex vivo modification of cells for reintroduction into a mammal. As for other methods of delivering genetic vaccines, if necessary, vaccine administration is repeated in order to maintain the desired level of immunomodulation.

Genetic vaccine vectors (e.g., adenoviruses, liposomes, papillomaviruses, retroviruses, etc.) can be administered directly to the mammal for transduction of cells in vivo. The genetic vaccines obtained using the methods of the invention can be formulated as pharmaceutical compositions for administration in any suitable manner, including parenteral (e.g., subcutaneous, intramuscular, intradermal, or intravenous), topical, oral, rectal, intrathecal, buccal (e.g., sublingual), or local administration, such as by aerosol or transdermally, for prophylactic and/or therapeutic treatment. Pretreatment of skin, for example, by use of hair-removing agents, may be useful in transdermal delivery. Suitable methods of administering such packaged nucleic acids are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions of the present invention. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of genetic vaccine vector in these formulations can vary widely, and will be selected primarily based on fluid volumes, viscosities, body weight and the like in accordance with the particular mode of administration selected and the patient's needs.

Formulations suitable for oral administration can consist of (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, tragacanth, microcrystalline cellulose, acacia, gelatin, colloidal silicon dioxide, croscarmellose sodium, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, usually sucrose and acacia or tragacanth, as well as pastilles comprising the active ingredient in an inert base, such as gelatin and glycerin or sucrose and acacia emulsions, gels, and the like containing, in addition to the active ingredient, carriers known in the art. It is recognized that the genetic vaccines, when administered orally, must be protected from digestion. This is typically accomplished either by complexing the vaccine vector with a composition to render it resistant to acidic and enzymatic hydrolysis or by packaging the vector in an appropriately resistant carrier such as a liposome. Means of protecting vectors from digestion are well known in the art. The pharmaceutical compositions can be encapsulated, e.g., in liposomes, or in a formulation that provides for slow release of the active ingredient.

The packaged nucleic acids, alone or in combination with other suitable components, can be made into aerosol formulations (e.g., they can be “nebulized”) to be administered via inhalation. Aerosol formulations can be placed into pressurized acceptable propellants, such as dichlorodifluoromethane, propane, nitrogen, and the like.

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the packaged nucleic acid with a base, including, for example, liquid triglycerides, polyethylene glycols, and paraffin hydrocarbons.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration and intravenous administration are the preferred methods of administration. The formulations of packaged nucleic acid can be presented in unit-dose or multi-dose sealed containers, such as ampoules and vials.

Injection solutions and suspensions can be prepared from sterile powders, granules, and tablets of the kind previously described. Cells transduced by the packaged nucleic acid can also be administered intravenously or parenterally.

The dose administered to a patient, in the context of the present invention should be sufficient to effect a beneficial therapeutic response in the patient over time. The dose will be determined by the efficacy of the particular vector employed and the condition of the patient, as well as the body weight or vascular surface area of the patient to be treated. The size of the dose also will be determined by the existence, nature, and extent of any adverse side-effects that accompany the administration of a particular vector, or transduced cell type in a particular patient.

In determining the effective amount of the vector to be administered in the treatment or prophylaxis of an infection or other condition, the physician evaluates vector toxicities, progression of the disease, and the production of anti-vector antibodies, if any. In general, the dose equivalent of a naked nucleic acid from a vector is from about 1 μg to 1 mg for a typical 70 kilogram patient, and doses of vectors used to deliver the nucleic acid are calculated to yield an equivalent amount of therapeutic nucleic acid. Administration can be accomplished via single or divided doses.

In therapeutic applications, compositions are administered to a patient suffering from a disease (e.g., an infectious disease or autoimmune disorder) in an amount sufficient to cure or at least partially arrest the disease and its complications. An amount adequate to accomplish this is defined as a “therapeutically effective dose.” Amounts effective for this use will depend upon the severity of the disease and the general state of the patient's health. Single or multiple administrations of the compositions may be administered depending on the dosage and frequency as required and tolerated by the patient. In any event, the composition should provide a sufficient quantity of the proteins of this invention to effectively treat the patient.

In prophylactic applications, compositions are administered to a human or other mammal to induce an immune response that can help protect against the establishment of an infectious disease or other condition.

The toxicity and therapeutic efficacy of the genetic vaccine vectors provided by the invention are determined using standard pharmaceutical procedures in cell cultures or experimental animals. One can determine the LD₅₀ (the dose lethal to 50% of the population) and the ED₅₀ (the dose therapeutically effective in 50% of the population) using procedures presented herein and those otherwise known to those of skill in the art.

A typical pharmaceutical composition for intravenous administration would be about 0.1 to 10 mg per patient per day. Dosages from 0.1 up to about 100 mg per patient per day may be used, particularly when the drug is administered to a secluded site and not into the blood stream, such as into a body cavity or into a lumen of an organ. Substantially higher dosages are possible in topical administration. Actual methods for preparing parenterally administrable compositions will be known or apparent to those skilled in the art and are described in more detail in such publications as Remington's Pharmaceutical Science, 15th ed., Mack Publishing Company, Easton, Pa. (1980).

The genetic vaccines obtained using the methods of the invention can be packaged in packs, dispenser devices, and kits for administering genetic vaccines to a mammal. For example, packs or dispenser devices that contain one or more unit dosage forms are provided. Typically, instructions for administration of the compounds will be provided with the packaging, along with a suitable indication on the label that the compound is suitable for treatment of an indicated condition. For example, the label may state that the active compound within the packaging is useful for treating a particular infectious disease, autoimmune disorder, tumor, or for preventing or treating other diseases or conditions that are mediated by, or potentially susceptible to, a mammalian immune response.

### VII. Uses of Genetic Vaccines

Genetic vaccines which include optimized vector modules and other reagents provided by the invention are useful for treating many diseases and other conditions that are either mediated by a mammalian immune system or are susceptible to treatment by an appropriate immune response. Representative examples of these diseases are listed below; antigens appropriate for each are described in copending, commonly assigned U.S. patent application Ser. No. ______, filed Feb. 10, 1999 as TTC Attorney Docket No. 18097-028710US.

A. Infectious Diseases

Genetic vaccine vectors obtained according to the methods of the invention are useful in both prophylaxis and therapy of infectious diseases, including those caused by any bacterial, fungal, viral, or other pathogens of mammals. In some embodiments, protection is conferred by use of a genetic vaccine vector that will express an antigen (either or both of a humoral antigen or a T cell antigen) of the pathogen of interest. In preferred embodiments, the antigen is evolved using the methods of the invention in order to obtain optimized antigens as described herein. The vector induces an immune response against the antigen. One or several antigens or antigen fragments can be included in one genetic vaccine delivery vehicle. Examples of pathogens and corresponding polypeptides from which an antigen can be obtained include, but are not limited to, HIV (gp120, gp160), hepatitis B, C, D, E (surface antigen), rabies (glycoprotein), Schistosoma mansoni (calpain; Jankovic (1996) J. Immunol. 157: 806-14). Other pathogen infections that are treatable using genetic vaccine vectors include, for example, herpes zoster, herpes simplex -1 and -2, tuberculosis (including chronic, drug-resistant), lyme disease (Borrelia burgorferii), syphilis, parvovirus, rabies, human papillomavirus, and the like.

B. Inflammatory and Autoimmune Diseases

Autoimmune diseases are characterized by immune response that attacks tissues or cells of ones own body, or pathogen-specific immune responses that also are harmful for ones own tissues or cells, or non-specific immune activation which is harmful for ones own tissues or cells. Examples of autoimmune diseases include, but are not limited to, rheumatoid arthritis, SLE, diabetes mellitus, myasthenia gravis, reactive arthritis, ankylosing spondylitis, and multiple sclerosis. These and other inflammatory conditions, including IBD, psoriasis, pancreatitis, and various immunodeficiencies, can be treated using genetic vaccines that include vectors and other components obtained using the methods of the invention.

These conditions are often characterized by an accumulation of inflammatory cells, such as lymphocytes, macrophages, and neutrophils, at the sites of inflammation. Altered cytokine production levels are often observed, with increased levels of cytokine production. Several autoimmune diseases, including diabetes and rheumatoid arthritis, are linked to certain MHC haplotypes. Other autoimmune-type disorders, such as reactive arthritis, have been shown to be triggered by bacteria such as Yersinia and Shigella, and evidence suggests that several other autoimmune diseases, such as diabetes, multiple sclerosis, rheumatoid arthritis, may also be initiated by viral or bacterial infections in genetically susceptible individuals.

Current strategies of treatment generally include anti-inflammatory drugs, such as NSAID or cyclosporin, and antiproliferative drugs, such as methotrexate. These therapies are non-specific, so a need exists for therapies having greater specificity, and for means to direct the immune responses towards the direction that inhibits the autoimmune process.

The present invention provides several strategies by which these needs can be fulfilled. First, the invention provides methods of obtaining vaccines which exhibit improved delivery of tolerogenic antigens, antigens which have improved antigenicity, genetic vaccine-mediated tolerance, and modulation of the immune response by inclusion of appropriate accessory molecules. In a preferred embodiment, the vaccines prepared according to the invention exhibit improved induction of tolerance by oral delivery. Oral tolerance is characterized by induction of immunological tolerance after oral administration of large quantities of antigen (Chen et al. (1995) Science 265: 1237-1240; Haq et al. (1995) Science 268: 714-716). In animal models, this approach has proven to be a very promising approach to treat autoimmune diseases, and clinical trials are in progress to address the efficacy of this approach in the treatment of human autoimmune diseases, such as rheumatoid arthritis and multiple sclerosis (Chen et al. (1994) Science 265:1237-40; Whitacre et al. (1996) Clin. Immunol. Immunopathol. 80: S31-9; Hohol et al. (1996) Ann. N. Y. Acad. Sci. 778:243-50). It has also been suggested that induction of oral tolerance against viruses used in gene therapy might reduce the immunogenicity of gene therapy vectors. However, the amounts of antigen required for induction of oral tolerance are very high and improved methods for oral delivery of antigenic proteins would significantly improve the efficacy of induction of oral tolerance.

Expression library immunization (Barry et al. (1995) Nature 377: 632) is a particularly useful method of screening for optimal antigens for use in genetic vaccines. For example, to identify autoantigens present in Yersinia, Shigella, and the like, one can screen for induction of T cell responses in HLA-B27 positive individuals. Complexes that include epitopes of bacterial antigens and MHC molecules associated with autoimmune diseases, e.g., HLA-B27 in association with Yersinia antigens can be used in the prevention of reactive arthritis and ankylosing spondylitis in HLA-B27 positive individuals.

Treatment of autoimmune and inflammatory conditions can involve not only administration of tolerogenic antigens, but also the use of a combination of cytokines, costimulatory molecules, and the like. Such cocktails are formulated for induction of a favorable immune response, typically induction of autoantigen-specific tolerance. Cocktails can also include, for example, CD1, which is crucially involved in recognition of self antigens by a subset of T cells (Porcelli (1995) Adv. Immunol. 59: 1). Genetic vaccine vectors and cocktails that skew immune responses towards the T_{H}2 are often used in treating autoimmune and inflammatory conditions, both with antigen-specific and antigen nonspecific vectors.

Screening of genetic vaccines and accessory molecules can be done in animal models which are known to those of skill in the art. Examples of suitable models for various conditions include collagen induced arthritis, the NFS/sld mouse model of human Sjogren's syndrome; a 120 kD organ-specific autoantigen recently identified as an analog of human cytoskeletal protein α-fodrin (Haneji et al. (1997) Science 276: 604), the New Zealand Black/White F1 hybrid mouse model of human SLE, NOD mice, a mouse model of human diabetes mellitus, fas/fas ligand mutant mice, which spontaneously develop autoimmune and lymphoproliferative disorders (Watanabe-Fukunaga et al. (1992) Nature 356: 314), and experimental autoimmune encephalomyelitis (EAE), in which myelin basic protein induces a disease that resembles human multiple sclerosis.

Autoantigens that are useful in genetic vaccines for treating multiple sclerosis include, but are not limited to, myelin basic protein (Stinissen et al. (1996) J. Neurosci. Res. 45: 500-511) or a fusion protein of myelin basic protein and proteolipid protein in multiple sclerosis (Elliott et al. (1996) J. Clin. Invest. 98: 1602-1612), proteolipid protein (PLP) (Rosener et al. (1997) J. Neuroimmunol. 75: 28-34), 2′,3′-cyclic nucleotide 3′-phosphodiesterase (CNPase) (Rosener et al. (1997) J. Neuroimmunol. 75: 28-34), the Epstein Barr virus nuclear antigen-1 (EBNA-1) in multiple sclerosis (Vaughan et al. (1996) J. Neuroimmunol. 69: 95-102), HSP70 in multiple sclerosis (Salvetti et al. (1996) J. Neuroimmunol. 65: 143-53; Feldmann et al. (1996) Cell 85: 307).

C. Allergy and Asthma

Genetic vaccine vectors and other reagents obtained using the methods of the invention can be used to treat allergies and asthma. Allergic immune responses are results of complex interactions between B cells, T cells, professional antigen-presenting cells (APC), eosinophils and mast cells. These cells take part in allergic immune responses both as modulators of the immune responses and are also involved in producing factors directly involved in initiation and maintenance of allergic responses.

Synthesis of polyclonal and allergen-specific IgE requires multiple interactions between B cells, T cells and professional antigen-presenting cells (APC). Activation of naive, unprimed B cells is initiated when specific B cells recognize the allergen by cell surface immunoglobulin (sIg). However, costimulatory molecules expressed by activated T cells in both soluble and membrane-bound forms are necessary for differentiation of B cells into IgE-secreting plasma cells. Activation of T helper cells requires recognition of an antigenic peptide in the context of MHC class II molecules on the plasma membrane of APC, such as monocytes, dendritic cells, Langerhans cells or primed B cells. Professional APC can efficiently capture the antigen and the peptide-MHC class II complexes are formed in a post-Golgi, proteolytic intracellular compartment and subsequently exported to the plasma membrane, where they are recognized by T cell receptor (TCR) (Monaco (1995) J. Leuk. Biol. 57: 543-547). In addition, activated B cells express CD80 (B7-1) and CD86 (B7-2, B70), which are the counter receptors for CD28 and which provide a costimulatory signal for T cell activation resulting in T cell proliferation and cytokine synthesis (Bluestone (1995) Immunity 2: 555-559). Since allergen-specific T cells from atopic individuals generally belong to the T_{H}2 cell subset, activation of these cells also leads to production of IL-4 and IL-13, which, together with membrane-bound costimulatory molecules expressed by activated T helper cells, direct B cell differentiation into IgE-secreting plasma cells (de Vries and Punnonen, In Cytokine Regulation of Humoral Immunity: Basic and Clinical Aspects, Ed. C M Snapper, John Wiley & Sons Ltd, West Sussex, UK, p. 195-215, 1996).

Mast cells and eosinophils are key cells in inducing allergic symptoms in target organs. Recognition of specific antigen by IgE bound to high-affinity IgE receptors on mast cells, basophils or eosinophils results in crosslinking of the receptors leading to degranulation of the cells and rapid release of mediator molecules, such as histamine, prostaglandins and leukotrienes, causing allergic symptoms.

Immunotherapy of allergic diseases currently includes hyposensibilization treatments using increasing doses of allergen injected to the patient. These treatments result skewing of immune responses towards T_{H}1 phenotype and increase the ratio of IgG/IgE antibodies specific for allergens. Because these patients have circulating IgE antibodies specific for the allergens, these treatments include significant risk of anaphylactic reactions. In these reactions, free circulating allergen is recognized by IgE molecules bound to high-affinity IgE receptors on mast cells and eosinophils. Recognition of the allergen results in crosslinking of the receptors leading to release of mediators, such as histamine, prostaglandins, and leukotrienes, which cause the allergic symptoms, and occasionally anaphylactic reactions. Other problems associated with hyposensibilization include low efficacy and difficulties in producing allergen extracts reproducibly.

Genetic vaccines provide a means of circumventing the problems that have limited the usefulness of previously known hyposensibilization treatments. For example, by expressing antigens on the surface of cells, such as muscle cells, the risk of anaphylactic reactions is significantly reduced. This can be achieved by using genetic vaccine vectors that encode transmembrane forms of allergens. The allergens can also be modified in such a way that they are efficiently expressed in transmembrane forms, further reducing the risk of anaphylactic reactions. Another advantage provided by the use of genetic vaccines for hyposensibilization is that the genetic vaccines can include cytokines and accessory molecules which further direct the immune responses towards the T_{H}1 phenotype, thus reducing the amount of IgE antibodies produced and increasing the efficacy of the treatments. Vectors can also be evolved to induce primarily IgG and IgM responses, with little or no IgE response. Furthermore, DNA shuffling can be used to generate allergens that are not recognized by the specific IgE antibodies preexisting in vivo, yet are capable of inducing efficient activation of allergen-specific T cells. For example, using phage display selection, one can express shuffled allergens on phage, and only those that are not recognized by specific IgE antibodies are selected. These are further screened for their capacity to induce activation of specific T cells. An efficient T cell response is an indication that the T cell epitopes are functionally intact, although the B cell epitopes were altered, as indicated by lack of binding of specific antibodies.

In these methods, polynucleotides encoding known allergens, or homologs or fragments thereof (e.g., immunogenic peptides) are inserted into DNA vaccine vectors and used to immunize allergic and asthmatic individuals. DNA shuffling can be used to obtain antigens that activate T cells but cannot induce anaphylactic reactions. Examples of allergies that can be treated include, but are not limited to, allergies against house dust mite, grass pollen, birch pollen, ragweed pollen, hazel pollen, cockroach, rice, olive tree pollen, fungi, mustard, bee venom.

The invention also provides a solution to another shortcoming of genetic vaccination as a treatment for allergy and asthma. While genetic vaccination primarily induces CD8³⁰ T cell responses, induction of allergen-specific IgE responses is dependent on CD4⁺T cells and their help to B cells. T_{H}2-type cells are particularly efficient in inducing IgE synthesis because they secrete high levels of IL-4, IL-5 and IL-13, which direct Ig isotype switching to IgE synthesis. IL-5 also induces eosinophilia. The methods of the invention can be used to develop genetic vaccines that efficiently induce CD4⁺T cell responses, and direct differentiation of these cells towards the T_{H}1 phenotype.

The invention also provides methods by which the level of antigen release by a genetic vaccine vector is regulated. Regulation of the antigen dose is crucial at the onset of hyposensibilization for safety reasons. Low antigen levels are preferably used at first, with the antigen level increasing once evidence has been obtained that the antigen does not induce adverse effects in the individual. The DNA shuffling methods of the invention allow generation of genetic vaccine vectors that induce expression of different (high and low) levels of antigen. For example, two or more different evolved promoters can be used for antigen expression. Alternatively, the antigen gene itself can be evolved for different levels of expression by, for example, altering codon usage. Vectors that induce different levels of antigen expression can be screened by use of specific monoclonal antibodies, and cell sorting (e.g, FACS).

D. Cancer

Immunotherapy has great promise for the treatment of cancer and prevention of metastasis. By inducing an immune response against cancerous cells, the body's immune system can be enlisted to reduce or eliminate cancer. Genetic vaccines prepared using the methods of the invention, as well as accessory molecules described herein, provide cancer immunotherapies of increased effectiveness compared to those that are presently available.

One approach to cancer immunotherapy is vaccination using genetic vaccines that encode antigens that are specific for tumor cells. The methods of the invention can be used for enhancement of immune responses against known tumor-specific antigens, and also to search for novel protective antigenic sequences. Genetic vaccines that exhibit optimized antigen expression, processing, and presentation can be obtained as described herein. The methods of the invention are also suitable for obtaining optimized cytokines, costimulatory molecules, and other accessory molecules that are effective in induction of an antitumor immune response, as well as for obtaining genetic vaccines and cocktails that include these and other components present in optimal combinations. The approach used for each particular cancer can vary. For treatment of hormone-sensitive cancers (for example, breast cancer and prostate cancer), methods of the invention can be used to obtain optimized hormone antagonists. For highly immunogenic tumors, including melanoma, one can screen for genetic vaccine vectors that optimally boost the immune response against the tumor. Breast cancer, in contrast, is of relatively low immunogenicity and exhibits slow progression, so individual treatments can be designed for each patient. Prevention of metastasis is also a goal in design of genetic vaccines.

### EXAMPLES

The following examples are offered to illustrate, but not to limit the present invention.

### Example 1

### Animal Model for Screening Genetic Vaccine Vectors

This Example provides a mouse model system that is useful for screening and testing genetic vaccine vectors in human skin in vivo. Pieces of human skin are xenotransplanted onto the back of SCID mice. Pieces of human skin can be obtained from infants undergoing circumcision, from skin removal operations due to, for example, cosmetic reasons, or from patients undergoing amputation due to, for example, accidents. These pieces are then transplanted onto the backs of C.B-17 scid/scid (SCID) mice as described by others (Deng et al. (1997) Nature Biotechnology 15: 1388-1391; Khavari et al. (1997) Adv. Clin. Res. 15:27-35; Choate and Khavari (1997) Human Gene Therapy 8:895-901).

The vector libraries are selected, for example, after topical application to the skin. However, in an analogous manner, depending on the optimal route of immunization, the evolved vectors can also be selected after i.m., i.v., i.d., oral, anal or vaginal delivery. The DNA delivered onto the skin can be in the form of a patch, in a form of a cream, in a form of naked DNA or mixture of DNA and transfection-enhancing agent (such as proteases, lipases or lipids/liposomes), and it can be applied after mechanical abrasion, after removal of the hair, or simply by adding a droplet of DNA or DNA-lipid/liposome mixture onto the skin. Similar delivery methods apply to small animals, such as mice or rat, large animals, such as cat, dog, cow, horse or monkey, as well as humans.

Suitable proteases and lipases that enhance the delivery include, but are not limited to, individuals or mixtures of the following: a protease (such as Alcalase or Savinase) with or without an alpha-amylase, a lipase (such as Lipolase) (Sarlo et al. (1997) J. Allergy Clin. Immunol. 100:480-7).

The recovery of the optimal vectors can be done from the transfected cells by, for example, PCR, or by recovering entire vectors. One can either select vectors based purely on their capacity to enter the cells or by selecting only cells that express the antigen encoded by the vector in normal mice, monkeys or SCID mice transplanted with human skin. One can use, for example, GFP as a marker gene, and after delivery detect cells that are transfected by fluorescence microscopy or flow cytometry. The positive cells can be isolated for example by flow cytometry based cell sorting. This format allows selection of vectors that optimally express antigens in and transfect human cells in vivo.

Additionally, one can screen in mice by selecting vectors that are able to induce effective immune responses after delivery onto the skin. One can select vectors that induce highest specific antibody or CTL responses, or one can select based on induction of protective immune response following challenge by the corresponding pathogen.

### Example 2

### Episomally-Replicating Nucleic Acid Vaccine Vector

This Example describes a procedure for obtaining stable, episomally maintained genetic vaccine vectors by applying DNA shuffling to human papillomavirus (HPV) genes. HPV can be maintained in human skin for extended periods (Bernard and Apt (1994) Arch. Dermatol. 130: 210). Despite these in vivo properties, it has not been possible to maintain HPV episomally in tissue culture due to underreplication. The primary goal of the procedure described in this Example is to improve the stability and copy number of vector constructs. Screening for natural HPV variants using traditional approaches or attempts to rationally design mutants with improved properties would require many person years of research.

To obtain improved mutants in an efficient manner, family shuffling is performed using the HPV E1 and E2 genes from different, but closely related, benign HPVs. Family shuffling allows one to generate and screen orders of magnitude more diversity than traditional mutagenesis approaches in much shorter time periods than are required for the traditional approaches. Libraries of HPV E1 and E2 genes are generated by using family shuffling of three closely related cutaneous HPV strains (HPV 2, 27, and 57). Alternatively, large libraries of vector sequences are generated by incorporation of random DNA sequences, for example derived from human or mouse genomic DNA, into genetic vaccine vectors. Green fluorescent protein (GFP) is used as a marker gene to detect the most stable vectors with superior expression levels. The best chimeric constructs from a library of millions of vectors are selected by flow cytometry-based cell sorting. Episomal vectors are then recovered providing an additional selection pressure towards nonintegrating vectors.

Initial screening is performed in cell culture, where processing of large libraries of shuffled material is feasible. Stable episomal vectors are also likely to prove to be very useful tools in other library screening applications. In contrast to randomly integrating and transient vectors, episomally maintained vectors result in high signal-to-noise ratios upon FACS selection, and they also significantly improve the possibility to recover the plasmids from a small number of selected cells. Alternatively or additionally, the vectors are screened and analyzed for durability in vivo in SCID mice transplanted with live human skin (see, Example 1).

To directly screen for optimal properties in human cells in vivo, the vector libraries are screened in an animal model, in which SCID mice are transplanted with human skin. In this model, live human skin is xenotransplanted onto the back of SCID mice without any signs of rejection, providing a possibility to optimize and evolve genetic vaccine vector directly in human tissue in vivo. Recursive selection of episomal vectors will provide strong selection pressure for vectors that remain episomal, yet provide a high level of gene expression. Moreover, despite their immunodeficient phenotype, SCID mice have normal levels of monocytes and macrophages. Therefore, antigen presenting cells (APC) derived from these mice can be used to assess the level of antigens delivered to professional antigen presenting cells, and to study the capacity of these cells to present antigens and induce activation of antigen-specific CD4³⁰ and CD8³⁰ T cells in vitro.

### Example 3

### Evolution Of The Major Immediate Early Promoter/Enhancer Region Of Cytomegalovirus

The major immediate-early (IE) region promoter/enhancer of cytomegalovirus (CMV) is widely used for regulating transcription of genes, because it is highly active in a broad range of cell types. An optimized CMV promoter (generated by DNA shuffling) which directs increased levels of gene expression, can improve the efficacy of genetic vaccines. The fact that the CMV promoter is active in human and animal cells means that it can be used to express foreign genes both in animal models and in clinical applications.

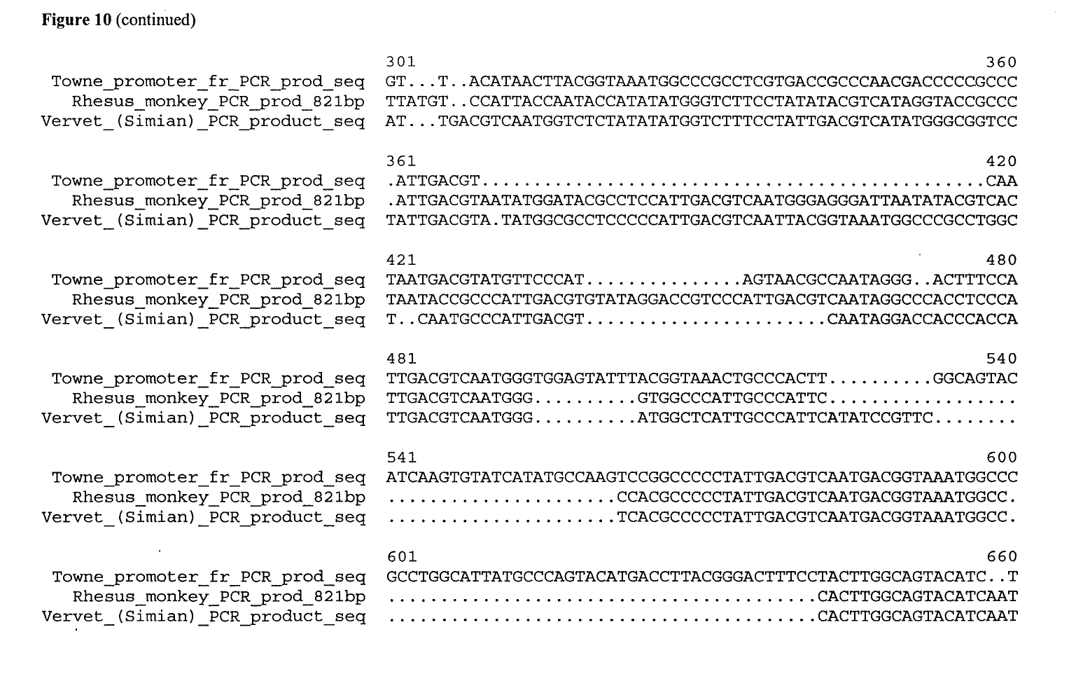

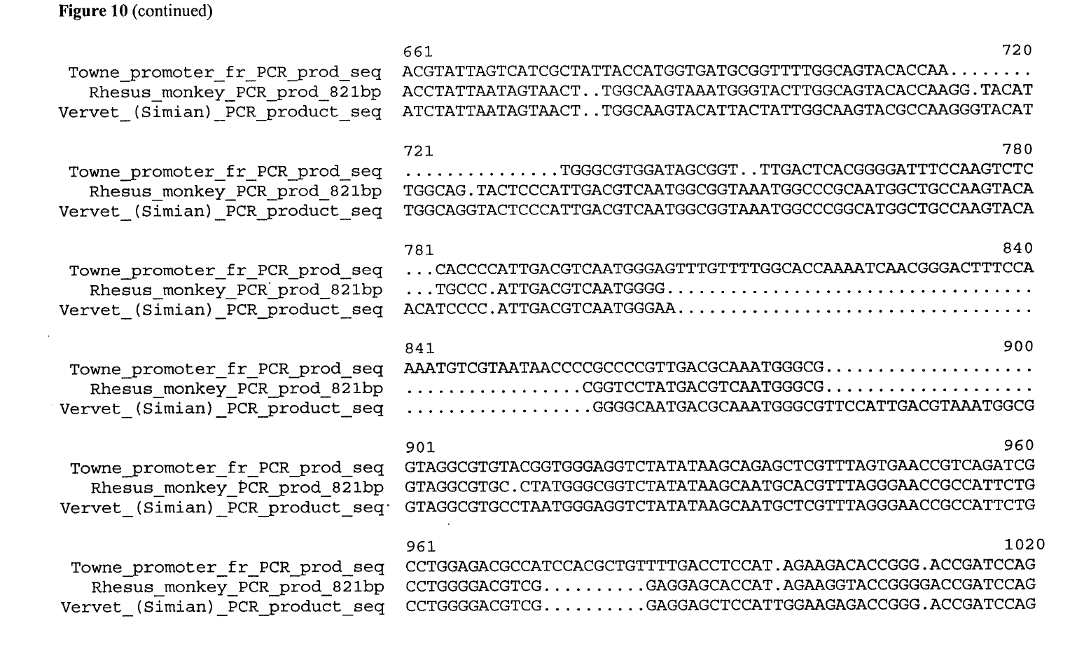

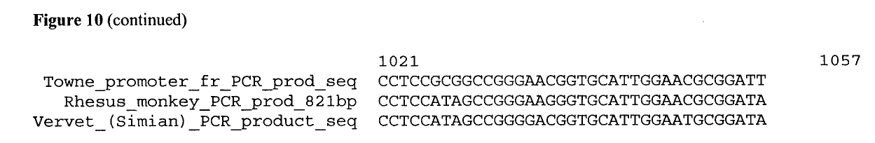

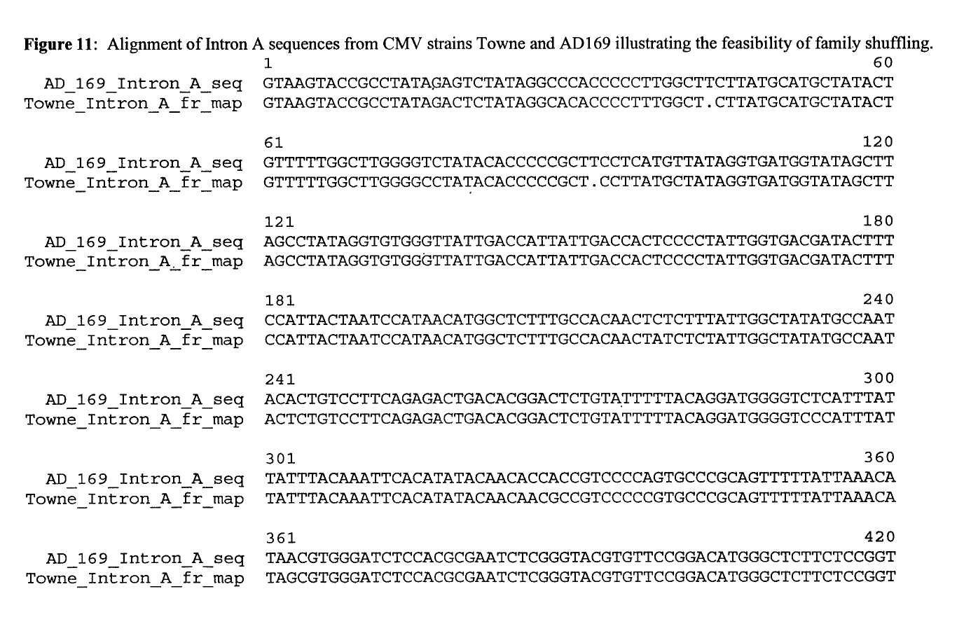

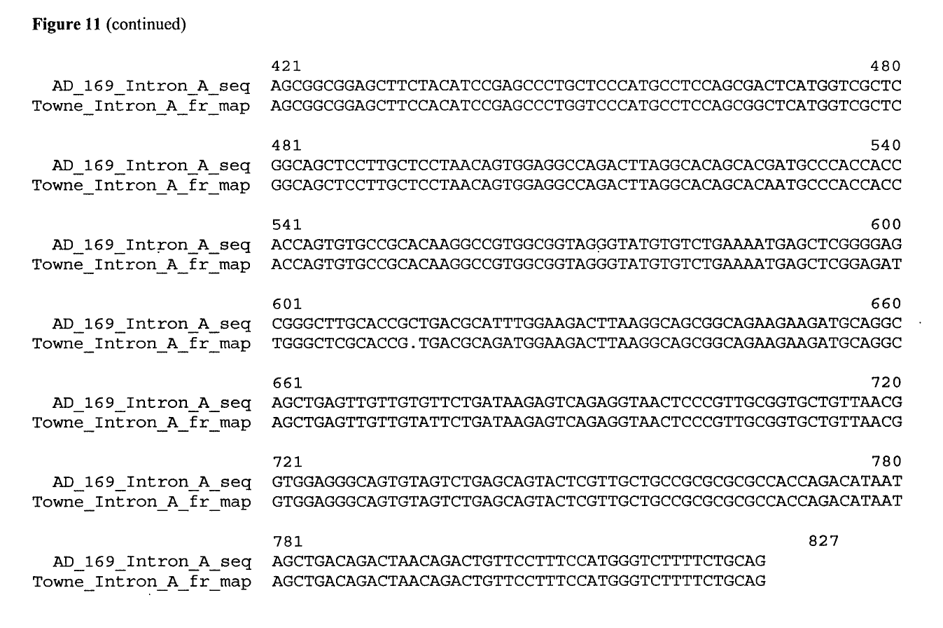

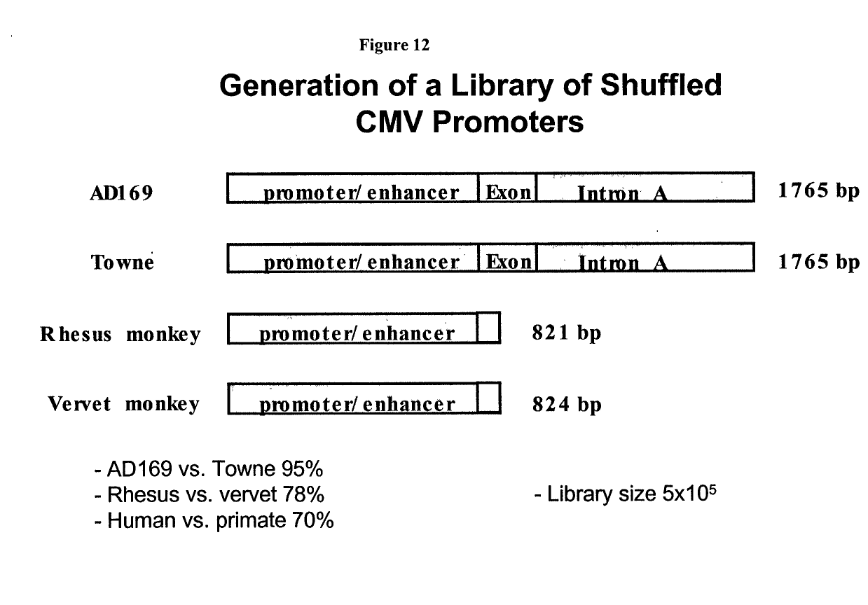

A library of chimeric promoter/enhancer sequences was created by DNA shuffling of wild-type sequences from four related strains of CMV. The promoter, enhancer and first intron sequences of the IE region are obtained by PCR from the AD169 and Towne human CMV strains, and from rhesus and vervet monkey CMVs (FIG. 12). The promoter/enhancer sequences of the human CMV strains are 95% identical, and share approximately 70% identity with the sequences of the monkey isolates, allowing the use of family shuffling to generate a library with greater diversity than would be achieved using the conventional shuffling procedure. Alignments and the sequence similarities of the promoter/enhancer regions of these sequences are shown in FIG. 10. Alignments and sequence similarities of the intron A sequences in the PCR products from the human CMV strains, Towne and AD 169 are shown in FIG. 11, and schematic diagrams of the PCR products obtained upon amplification of these promoters are shown in FIG. 12.

The following primers can be used to amplify promoter sequences from human and monkey CMVs:

Primers for promoters in human CMV strains Towne and AD169:

5′-primer: 5′-ATA TGA GGC TAT ATC GCC GAT A-3′

3′ primer: 5′-AAG GAC GGT GAC TGC AGA AAA-3′

Primers for Rhesus Monkey CMV promoter:

5′-primer: 5″-AAT GGC GAC TTG GCA TTG AGC CAA TT-3″

3′ primer: 5′-TAT CCG CGT TCC AAT GCA CCC TT-3′

Primers for Vervet Monkey CMV promoter:

5′-primer: 5′-ACT TGG CAC GGT GCC AAG TTT-3′

3′ primer: 5′-TAT CCG CAT TCC AAT GCA CCG T-3′

Following shuffling, the library was cloned into a plasmid backbone and used to direct transcription of a marker gene in mammalian cells. An internal marker under the control of a native promoter was included in the plasmid vector, enabling analysis and selection of cells expressing equal numbers of vectors. An example of a suitable vector for use in screening shuffled promoter sequences is shown in FIG. 7.

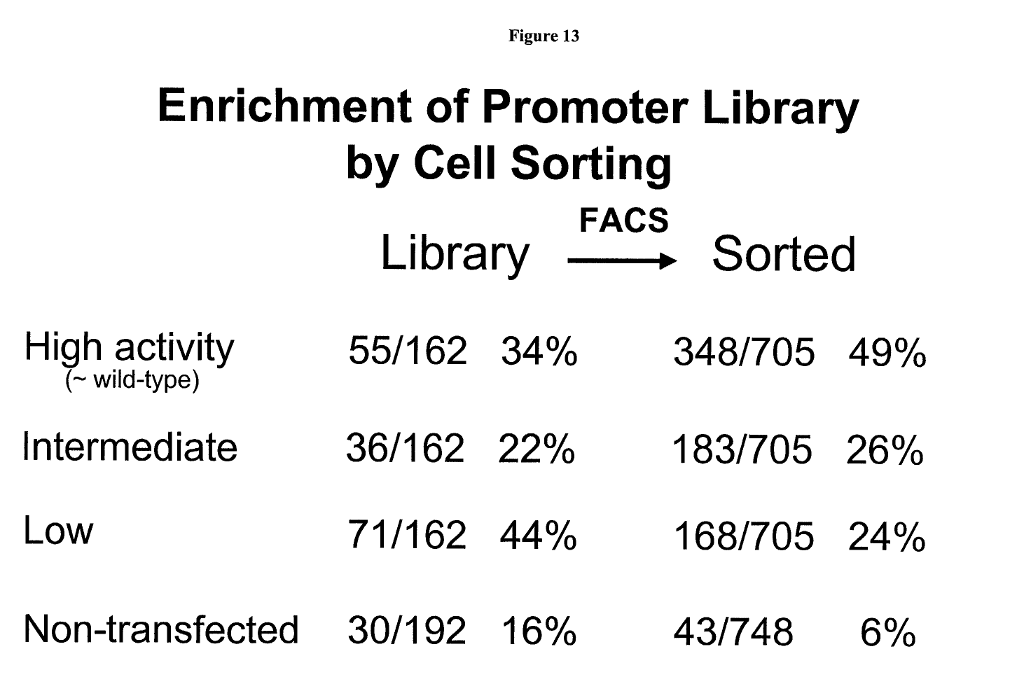

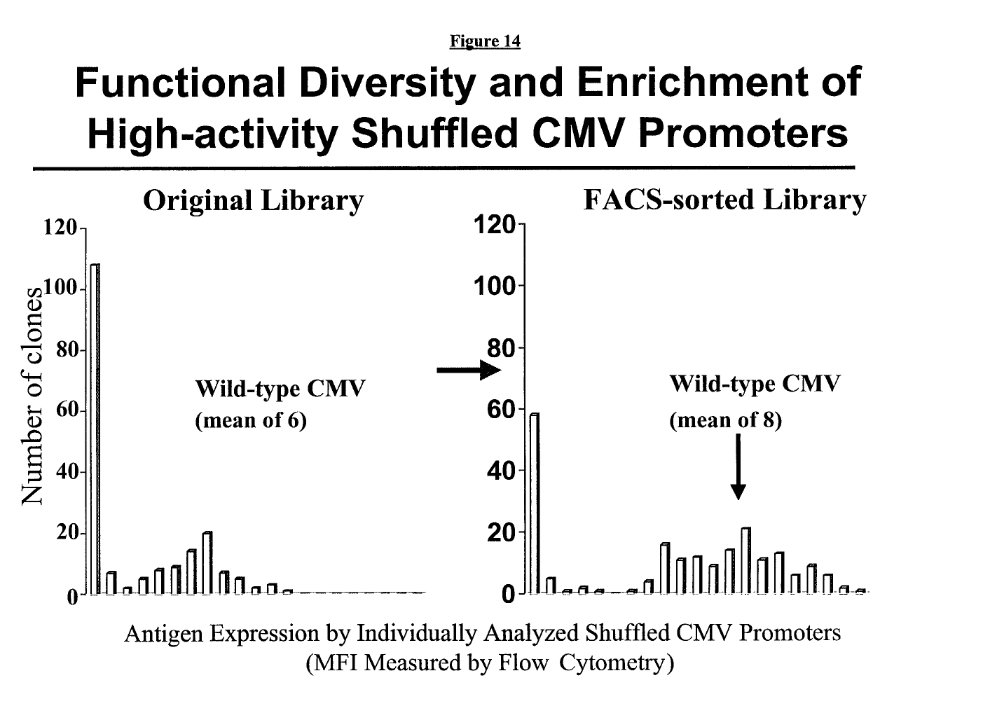

The transfected cells were screened by flow cytometric cell sorting to identify those which express highest levels of the marker gene, normalized against the internal marker to account for differences in vector copy numbers per cell. Vectors carrying optimal promoter sequences are then recovered and subjected to further cycles of shuffling and selection. Results shown in FIG. 13 demonstrate that recombination followed by fluorescence-activated cell sorting resulted in the promoter library being enriched for promoters having strong activity. FIG. 14 shows the distribution of antigen expression, as measured by flow cytometry, of individually analyzed shuffled clones. Again, the FACS-sorted library enriched the population for high-activity promoters.

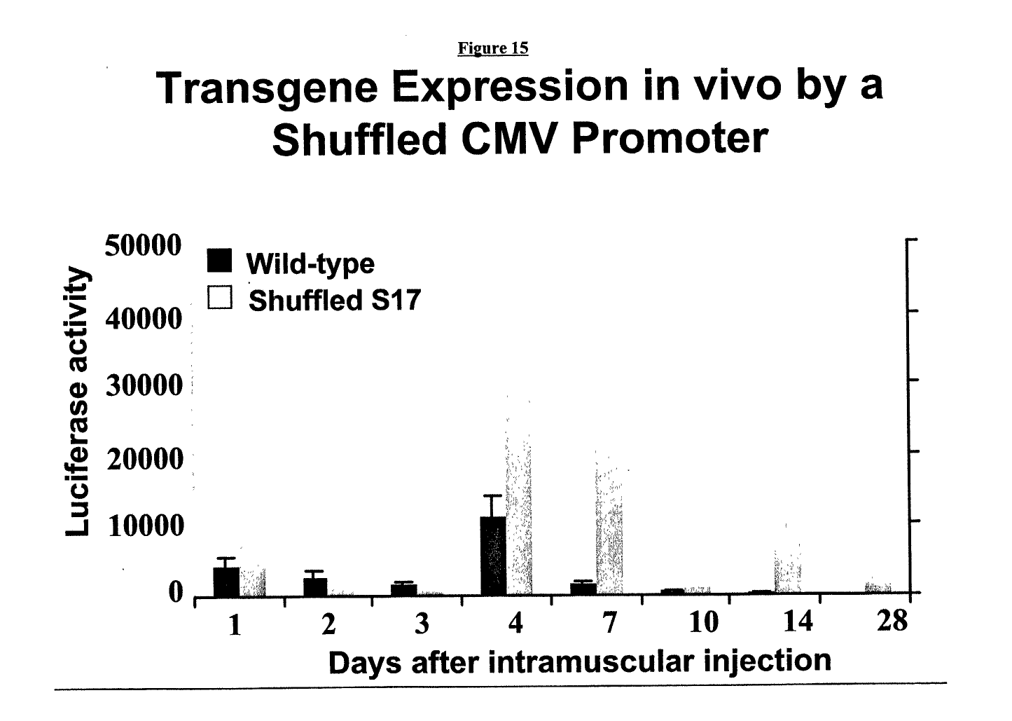

A Vector That Contains a Shuffled CMV Promoter (S17) Operably Linked To a Luciferase-encoding Gene Was Injected Intramuscularly Into a Mouse [??], And The Amount of Luciferase Expression Was Determined at Various Time Points After Injection. Results Are Shown in FIG. 15

### Example 4

### Shuffling Of Oligo-Directed Cpg Knock-Outs

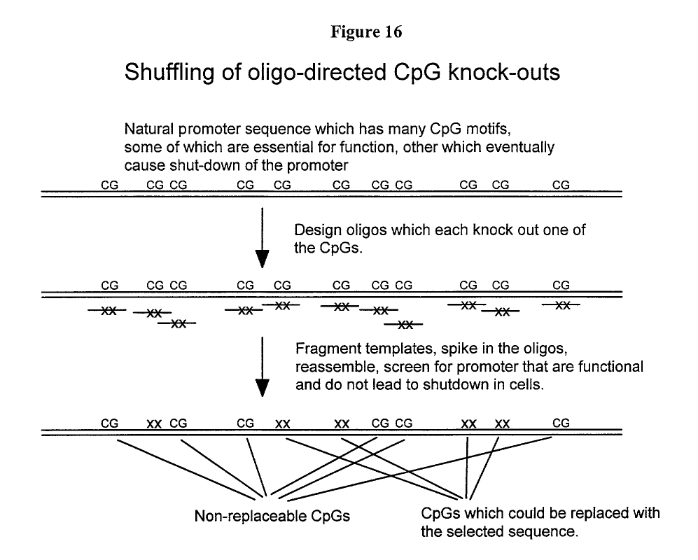

A common problem associated with genetic vaccine vectors is that the expression induced by the vectors is often short-lasting due to downregulation of promoter activity. One reason for downregulation of promoter activities is methylation (Robertson and Ambinder (1997) 71:6445-54). CpG sequences are particularly prone to methylation and this example describes the use of DNA shuffling method to generate promoter sequences where all unnecessary CpG sequences have been deleted. The approach is illustrated in FIG. 16.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

What is claimed is:

1. A multicomponent genetic vaccine comprising two or more genetic vaccine components selected from the group consisting of:
a component that provides optimal antigen release;
a component that provides optimal production of cytotoxic T lymphocytes;
a component that directs release of an immunomodulator;
a component that directs release of a chemokine;
a component that facilitates binding to, or entry into, a desired target cell type;
a component that directs antigen peptides derived from uptake of an antigen into a cell to presentation on either Class I or Class II molecules.

2. The genetic vaccine of claim 1, wherein each component is present on a separate vector.

3. The genetic vaccine of claim 1, wherein each component-is present on the same vector.

4. The genetic vaccine of claim 3, wherein the vector is assembled by assembly PCR using as templates DNA fragments including a) a fragment which contains the first optimized recombinant genetic vaccine component and b) a separate DNA fragment which contains the second optimized recombinant genetic vaccine component.

5. The genetic vaccine of claim 1, which comprises a component designed for optimal antigen release that improves binding to, and uptake of, the genetic vaccine to target antigen-expressing cells.

6. The genetic vaccine of claim 5, wherein the target antigen-expressing cells are selected from the group consisting of myocytes and epithelial cells.

7. The genetic vaccine of claim 1, wherein the component confers optimal binding to, and uptake by, a target antigen presenting cell.

8. The genetic vaccine of claim 7, wherein the target antigen presenting cells are selected from the group consisting of dendritic cells, monocytes/macrophages, and Langerhan's cells.

9. The genetic vaccine of claim 1, wherein the component directs antigen peptides derived from uptake of an antigen into a cell to presentation on either Class I or Class II molecules.

10. The genetic vaccine of claim 9, wherein the component directs antigen peptides to presentation on Class I molecules and comprises a polynucleotide that encodes a protein selected from the group consisting of tapasin, TAP-1 and TAP-2.

11. The genetic vaccine of claim 9, wherein the component directs antigen peptides to presentation on Class II molecules and comprises a polynucleotide that encodes an endosomal or lysosomal protease.

12. The genetic vaccine of claim 1, wherein the desired target cell type is a dendritic cell or a Langerhans cell.

13. The genetic vaccine of claim 1, wherein the vaccine comprises:
a component for optimal antigen release;
a component optimized for CTL activation via dendritic cell presentation of antigen peptide on MHC Class I;
a component optimize d for release of IL-12 and IFNγ from resident tissue macrophages; and
a component optimized for recruitment of T_{H} cells to an immunization site.

14. The genetic vaccine of claim 1, wherein one or more of the components is obtained by a method comprising:
(1) recombining at least first and second forms of a nucleic acid which can confer a desired property upon a genetic vaccine, wherein the first and second forms differ from each other in two or more nucleotides, to produce a library of recombinant nucleic acids; and
(2) screening the library to identify at least one optimized recombinant component that exhibits an enhanced capacity to confer the desired property upon the genetic vaccine.

15. The genetic vaccine of claim 14, wherein the method used to obtain one or more of the components further comprises:
(3) recombining at least one optimized recombinant component with a further form of the nucleic acid, which is the same or different from the first and second forms, to produce a further library of recombinant nucleic acids;
(4) screening the further library to identify at least one further optimized recombinant component that exhibits an enhanced capacity to confer the desired property upon the genetic vaccine; and
(5) repeating (3) and (4), as necessary, until the further optimized recombinant component exhibits a further enhanced capacity to confer the desired property upon the genetic vaccine.

16. The genetic vaccine of claim 14, wherein the first form of the nucleic acid comprises a first member of a gene family and the second form comprises a second member of the gene family.

17. The genetic vaccine of claim 14, wherein the optimized recombinant component is backcrossed by:
recombining the optimized recombinant component with a molar excess of one or both of the first and second forms, to produce a further library of recombinant nucleic acids; and
screening the further library to identify at least one optimized recombinant component that exhibits a further enhanced capacity to confer the desired property upon the genetic vaccine.

18. The genetic vaccine of claim 16, wherein the first member of the gene family is obtained from a first species of organism and the second member of the gene family is obtained from a second species of organism.

19. The genetic vaccine of claim 14, wherein the genetic vaccine comprises DNA.

20. The genetic vaccine of claim 14, wherein the genetic vaccine comprises RNA.

21. The genetic vaccine of claim 14, wherein the genetic vaccine comprises a viral vector or a plasmid vector.

22. The genetic vaccine of claim 21, wherein the viral vector is selected from the group consisting of adenoviral, retroviral, papillomavirus, adenoassociated, and herpes viral vectors.

23. A method of obtaining a genetic vaccine component that confers upon a genetic vaccine vector an enhanced ability to replicate in a host cell, the method comprising:
creating a library of recombinant nucleic acids by subjecting to recombination at least two forms of a polynucleotide that can confer episomal replication upon a vector that contains the polynucleotide;
introducing into a population of host cells a library of vectors, each of which contains a member of the library of recombinant nucleic acids and a polynucleotide that encodes a cell surface antigen;
propagating the population of host cells for multiple generations; and
identifying cells which display the cell surface antigen on a surface of the cell, wherein cells which display the cell surface antigen are likely to harbor a vector that contains a recombinant vector module which enhances the ability of the vector to replicate episomally.

24. The method of claim 23, wherein the cells which display the cell surface antigen on a surface of the cell are identified by flow cytometry-based cell sorting.

25. A method of obtaining a genetic vaccine component which confers upon a vector an enhanced ability to replicate in a host cell, the method comprising:
creating a library of recombinant nucleic acids by subjecting to recombination at least two forms of a polynucleotide derived from a human papillomavirus that can confer episomal replication upon a vector that contains the polynucleotide;
introducing a library of vectors, each of which contains a member of the library of recombinant nucleic acids, into a population of host cells;
propagating the host cells for a plurality of generations; and
identifying cells that contain the vector.

26. The method of claim 25, wherein the polynucleotide comprises either or both of the human papillomavirus E1 and E2 genes.

27. A method of obtaining a genetic vaccine component that confers upon a vector an enhanced ability to replicate in a human host cell, the method comprising:
creating a library of recombinant nucleic acids by subjecting to recombination at least two forms of a polynucleotide that can confer episomal replication upon a vector that contains the polynucleotide;
introducing a library of genetic vaccine vectors, each of which comprises a member of the library of recombinant nucleic acids, into a test system that mimics a human immune response; and
determining whether the genetic vaccine vector replicates or induces an immune response in the test system.

28. The method of claim 27, wherein the test system comprises human skin cells present as a xenotransplant on skin of an immunocompromised non-human host animal.

29. The method of claim 28, wherein the host animal is a mouse.

30. The method of claim 28, wherein the host animal is transiently immunocompromised.

31. The method of claim 27, wherein test system comprises a non-human mammal that comprises a functional human immune system and replication is detected by determining whether the animal exhibits an immune response against the antigen.

32. The method of claim 31, wherein the non-human mammal that comprises a functional human immune system is obtained by introducing into an immunodeficient non-human mammal one or more of a human fetal tissue selected from the group consisting of liver, thymus, and bone marrow.

33. A method of obtaining a recombinant genetic vaccine component that confers upon a genetic vaccine an enhanced ability to induce a desired immune response in a mammal, the method comprising:
(1) recombining at least first and second forms of a nucleic acid which comprise a genetic vaccine vector, wherein the first and second forms differ from each other in two or more nucleotides, to produce a library of recombinant genetic vaccine vectors;
(2) transfecting the library of recombinant vaccine vectors into a population of mammalian cells selected from the group consisting of peripheral blood T cells, T cell clones, freshly isolated monocytes/macrophages and dendritic cells;
(3) staining the cells for the presence of one or more cytokines and identifying cells which exhibit a cytokine staining pattern indicative of the desired immune response; and
(4) obtaining recombinant vaccine vector nucleic acid sequences from the cells which exhibit the desired cytokine staining pattern.

34. The method of claim 33, wherein the desired immune response is a T_{H}1 response and the cells exhibit high levels of either or both of IL-2 and IFN-γ but low levels of one or more of IL-4, IL-5 and IL-13.

35. The method of claim 33, wherein the cells are selected from the group consisting of monocytes, macrophages, and dendritic cells and the desired immune response is a high or low level of cytokine production by the cells.

36. The method of claim 35, wherein the cytokine expressed at a high level is one or more selected from the group consisting of IL-6, IL-10, IL-12 and TNF-α.

37. A method of improving the ability of a genetic vaccine vector to modulate an immune response, the method comprising:
(1) recombining at least first and second forms of a nucleic acid which comprise a genetic vaccine vector, wherein the first and second forms differ from each other in two or more nucleotides, to produce a library of recombinant genetic vaccine vectors;
(2) transfecting the library of recombinant genetic vaccine vectors into a population of antigen presenting cells; and
(3) isolating from the cells optimized recombinant genetic vaccine vectors which exhibit enhanced ability to modulate a desired immune response.

38. The method of claim 37, wherein the method further comprises:
(4) recombining at least one optimized recombinant vaccine vector with a further form of the genetic vaccine vector, which is the same or different from the first and second forms, to produce a further library of recombinant genetic vaccine vectors;
(5) transfecting the further library of recombinant genetic vaccine vectors into a population of antigen presenting cells;
(6) identifying optimized recombinant genetic vaccine vectors which exhibit enhanced ability to modulate a desired immune response; and
(7) repeating (4) through (6), as necessary, to obtain a further optimized recombinant genetic vaccine vector which has a further enhanced ability to modulate a desired immune response.

39. The method of claim 37, wherein the antigen presenting cell is selected from the group consisting of a dendritic cell, a B lymphocyte, a monocyte, a macrophage cell, and a Langerhans cell.

40. The method of claim 37, wherein the optimized recombinant genetic vaccine vectors exhibit improved ability to enter an antigen presenting cell and are obtained by:
after the transfection step, washing the cells to remove vectors which did not enter an antigen presenting cell;
culturing the cells for a predetermined time after transfection;
lysing the antigen presenting cells; and
isolating the optimized recombinant genetic vaccine vector from the cell lysate.

41. The method of claim 37, wherein APCs that contain an optimized recombinant genetic vaccine vectors are identified by detecting expression of a marker gene that is included in the vectors.

42. The method of claim 41, wherein the marker gene encodes a cell surface antigen.

43. The method of claim 42, wherein expression of the marker gene is detected by flow cytometric cell sorting.

44. The method of claim 37, wherein the genetic vaccine vector comprises a nucleotide sequence that encodes an immunogenic antigen and optimized recombinant genetic vaccine vectors are identified by:
transfecting individual library members into separate cultures of antigen presenting cells;
co-culturing transfected APCs with T lymphocytes obtained from the same individual as the APCs; and
identifying transfected APC cultures which are capable of inducing a T lymphocyte response.

45. The method of claim 44, wherein the T lymphocyte response is selected from the group consisting of increased T lymphocyte proliferation, increased T lymphocyte-mediated cytolytic activity against a target cell, and increased cytokine production.

46. The method of claim 45, wherein the genetic vaccine vector is capable of inducing a T_{H}1 response as evidenced by the transfected APCs inducing a T lymphocyte response that involves one or more of proliferation, IL-2 production, and interferon-γ production.

47. The method of claim 44, wherein the optimized recombinant genetic vaccine vectors are identified by its improved capacity to induce an immune response in a test animal, wherein the immune response is selected from the group consisting of:
improved protection of the test animal against challenge infection;
improved production of specific antibodies in the test animal; and
improved activation of T lymphocytes in the test animal.

48. The method of claim 47, wherein the test animal is a mouse or a monkey.

49. The method of claim 44, wherein T lymphocytes are selected from the group consisting of CD4⁺T lymphocytes, CD8³⁰ T lymphocytes, and a mixture thereof.

50. The method of claim 37, wherein the genetic vaccine vector comprises a nucleotide sequence that encodes an antigen and optimized recombinant vaccine vectors are identified by:
injecting the library of recombinant genetic vaccine vectors into a test animal;
obtaining lymphatic cells from the test animal; and
recovering recombinant genetic vaccine vectors from the lymphatic cells, wherein the recovered recombinant genetic vaccine vectors exhibit improved ability to enter lymphatic cells.

51. The method of claim 50, wherein the lymphatic cells are dendritic cells.

52. The method of claim 50, wherein the antigen is a cell surface antigen and prior to isolating the optimized recombinant genetic vaccine vectors cells that contain an optimized recombinant vector are purified by binding to an affinity reagent which selectively binds to the cell surface antigen.

53. A method of obtaining a recombinant genetic vaccine vector which has an enhanced ability to induce a desired immune response in a mammal upon administration to the skin of the mammal, the method comprising:
(1) recombining at least first and second forms of a nucleic acid which comprise a genetic vaccine vector, wherein the first and second forms differ from each other in two or more nucleotides, to produce a library of recombinant genetic vaccine vectors;
(2) topically applying the library of recombinant genetic vaccine vectors to skin of a mammal;
(3) identifying vectors that induce an immune response; and
(4) recovering genetic vaccine vectors from the skin cells which contain vectors that induce an immune response.

54. The method of claim 53, wherein the immune response is a protective immune response.

55. The method of claim 53, wherein the immune response is a CTL response, a T helper cell response, or an antibody response.

56. A method of inducing an immune response in a mammal, the method comprising topically applying to skin of the mammal a genetic vaccine vector, wherein the genetic vaccine vector is optimized for topical application through use of DNA shuffling.

57. The method of claim 56, wherein the genetic vaccine vector is administered as a formulation selected from the group consisting of a transdermal patch, a cream, naked DNA, a mixture of DNA and a transfection-enhancing agent.

58. The method of claim 57, wherein the transfection-enhancing agent is one or more agents selected from the group consisting of a lipid, a liposome, a protease, and a lipase.

59. The method of claim 56, wherein the genetic vaccine vector is administered after pretreatment of the skin by abrasion or hair removal.

60. A method of obtaining an optimized genetic vaccine component that confers upon a genetic vaccine containing the component an enhanced ability to induce or inhibit apoptosis of a cell into which the vaccine is introduced, the method comprising:
(1) recombining at least first and second forms of a nucleic acid which comprise a nucleic acid that encodes an apoptosis-modulating polypeptide, wherein the first and second forms differ from each other in two or more nucleotides, to produce a library of recombinant nucleic acids;
(2) transfecting the library of recombinant nucleic acids into a population of mammalian cells;
(3) staining the cells for the presence of a cell membrane change which is indicative of apoptosis initiation; and
(4) obtaining recombinant apoptosis-modulating genetic vaccine components from the cells which exhibit the desired apoptotic membrane changes.

61. The method of claim 60, wherein the genetic vaccine component has an enhanced ability to induce apoptosis and the nucleic acids encode an apoptosis-inducing polypeptide.

62. The method of claim 61, wherein the apoptosis-inducing polypeptide is a Caspases polypeptide or a Fas polypeptide.

63. The method of claim 60, wherein the genetic vaccine component has an enhanced ability to inhibit apoptosis and the nucleic acids encode an apoptosis-inhibiting polypeptide.

64. The method of claim 63, wherein the apoptosis-inhibiting polypeptide is Bcl-2 or another Bcl-2 family member.

65. The method of claim 60, wherein the cell membrane change which is indicative of apoptosis initiation is translocation of phospholipid phosphatidylserine (PS) from the inner to the outer leaflet of the plasma membrane.

66. The method of claim 65, wherein the PS translocation is detected by increased or decreased binding of Annexin V.

67. A method of obtaining a genetic vaccine component that confers upon a genetic vaccine reduced susceptibility to a CTL immune response in a host mammal, the method comprising:
(1) recombining at least first and second forms of a nucleic acid which comprises a gene that encodes an inhibitor of a CTL immune response, wherein the first and second forms differ from each other in two or more nucleotides, to produce a library of recombinant CTL inhibitor nucleic acids;
(2) introducing genetic vaccine vectors which comprise the library of recombinant CTL inhibitor nucleic acids into a plurality of human cells;
(3) selecting cells which exhibit reduced MHC class I molecule expression; and
(4) obtaining optimized recombinant CTL inhibitor nucleic acids from the selected cells.

68. The method of claim 67, wherein the method further comprises:
(5) recombining at least one recombinant CTL inhibitor nucleic acid with a further form of the gene that encodes an inhibitor of a CTL immune response, which is the same or different from the first and second forms, to produce a further library of recombinant CTL inhibitor nucleic acids;
(6) introducing genetic vaccine vectors which comprise the library of recombinant CTL inhibitor nucleic acids into a plurality of human cells; and
(7) selecting cells which exhibit reduced MHC class I molecule expression, wherein the selected cells comprise recombinant genetic vaccine vectors which exhibit reduced susceptibility to a CTL immune response in a host mammal; and
(8) repeating (5) through (7), as necessary, to obtain a further optimized recombinant CTL inhibitor genetic vaccine component that confers upon a genetic vaccine a further reduced susceptibility to a CTL immune response in a host mammal.

69. The method of claim 67, wherein the nucleic acid comprises a gene that encodes an inhibitor of MHC class I-mediated antigen presentation.

70. The method of claim 69, wherein the gene is selected from the group consisting of US2, US3, US6 and US11 genes of cytomegalovirus, a gene encoding adenoviral E3 protein, a gene encoding herpes simplex ICP47 protein, and a gene encoding a tapasin antagonist.

71. The method of claim 67, wherein the genetic vaccine comprises a viral vector.

72. A method of obtaining a genetic vaccine component that confers upon a genetic vaccine reduced susceptibility to a CTL immune response in a host mammal, the method comprising:
(1) recombining at least first and second forms of a nucleic acid which comprises a gene that encodes an inhibitor of a CTL immune response, wherein the first and second forms differ from each other in two or more nucleotides, to produce a library of recombinant CTL inhibitor nucleic acids;
(2) introducing viral vectors which comprise the library of recombinant CTL inhibitor nucleic acids into mammalian cells;
(3) identifying mammalian cells which express a marker gene included in the viral vectors a predetermined time after introduction, wherein the identified cells are resistant to a CTL response; and
(4) recovering as the genetic vaccine component the recombinant CTL inhibitor nucleic acids from the identified cells.

73. The method of claim 72, wherein the genetic vaccine comprises a viral vector that is selected from the group consisting of papillomavirus, adenovirus, and retrovirus.
